(19) United States
(12) Patent Application Publication
(10) Pub. No.: US 2015/0011559 A1
(43) Pub. Date: Jan. 8, 2015

(54) ANTIMICROBIAL AGENTS

(71) Applicant: RUTGERS, THE STATE UNIVERSITY OF NEW JERSEY, New Brunswick, NJ (US)

(72) Inventors: Edmond J. LaVoie, New Brunswick, NJ (US); Ajit Parhi, New Brunswick, NJ (US); Daniel S. Pilch, New Brunswick, NJ (US); Yongzheng Zhang, North Brunswick, NJ (US); Malvika Kaul, Now Brunswick, NJ (US)

(21) Appl. No.: 14/371,946

(22) PCT Filed: Jan. 11, 2013
(86) PCT No.: PCT/US2013/021300

§ 371 Date: Jul. 11, 2014

Related U.S. Application Data
(60) Provisional application No. 61/586,592, filed on Jan. 13, 2012.

(51) Int. Cl. C07D 417/06; C07C 279/04; C07D 213/58; C07D 277/64; C07D 295/096; C07C 279/08; C07D 233/60; C07C 211/27; C07C 279/10; C07C 211/64; C07D 277/28

(52) U.S. Cl. CPC C07D 417/06; C07C 211/64; C07C 279/04; C07D 213/58; C07D 277/64; C07D 277/28; C07C 279/08; C07D 233/60; C07C 211/27; C07C 279/10; C07D 295/096
USPC 514/252.12; 514/643; 514/634; 514/357; 514/367; 514/365; 514/399; 514/650

(57) ABSTRACT

The invention provides methods of treating a bacterial infection in a mammal comprising administering to the mammal a compound of formula I:

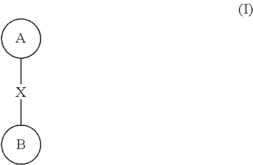

wherein A, B, and X have any of the meanings defined in the specification; or a pharmaceutically acceptable salt thereof, as well as novel compounds of formula I and salts thereof and pharmaceutical compositions comprising a compound of formula I or a pharmaceutically acceptable salt thereof.

### PRIORITY OF INVENTION

This application claims priority from U.S. Provisional Application No. 61/586,592 filed 13 Jan. 2012, which application is incorporated by reference.

### BACKGROUND OF THE INVENTION

The emergence of Multidrug Resistant (MDR) bacterial pathogens (e.g. methicillin-resistant Staphylococcus aureus (MRSA), Acinetobacter baumannii-calcoaceticus complex (ABC), etc.) has increased concerns as to the adequacy of current antimicrobials and pathogen treatment methods. The lethality of such pathogens, particularly MRSA, has often led to treatment methods that are experimental or would otherwise normally be avoided in standard clinical practice. For example, the antibiotic colistin was traditionally considered too nephrotoxic and neurotoxic for clinical use, but is nevertheless used to treat many MDR bacterial infections due to a paucity of available active drugs. The growing threat from MDR pathogens highlights a critical need for additional antimicrobials. In this connection, there is a pressing need for new antibiotics that exhibit novel mechanisms of action or that are able to circumvent known resistance pathways.

Elements of the bacterial cell division machinery present appealing targets for antimicrobial compounds because (i) they are essential for bacterial viability, (ii) they are widely conserved among bacterial pathogens, and (iii) they often have markedly different structures than their eukaryotic homologs. One such protein that has been identified as a potential target is the FtsZ protein. During the division process, FtsZ, along with approximately 15 other proteins, assemble at mid-cell into a large cell division complex (termed the divisome), ultimately facilitating cell cytokinesis. More importantly, FtsZ is widely conserved among many bacterial strains.

### SUMMARY OF THE INVENTION

In one embodiment the invention provides compounds that display antimicrobial activity. Accordingly, the invention provides a method for treating a bacterial infection in a mammal comprising administering to the mammal an effective amount of a compound of formula I:

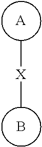

A is a ring selected from phenyl and thiazolyl, which ring is substituted with one or more (e.g. 1, 2, 3, or 4) R¹ and which ring is optionally substituted with one or more (e.g. 1, 2, 3, or 4) R^{b};

B is a ring selected from phenyl, pyridyl, benzothiazole, and thiazolyl, which ring is substituted with one or more (e.g. 1, 2, 3, or 4) R^{a} and which ring is optionally substituted with one or more (e.g. 1, 2, 3, or 4) R^{b};

R¹ is halo, —C(═O)NR^{v}R^{w}, phenyl, (C₁-C₆)alkyl, thiazolyl, R^{m}, or (C₃-C₆)cycloalkyl, which phenyl, (C₁-C₆)alkyl, thiazolyl, or (C₃-C₆)cycloalkyl is optionally substituted with one or more phenyl, thiazolyl or —C(═O)NR^{v}R^{w};

each R^{a} is independently:
a) —N⁺(R^{ac})₃Z⁻,
b) —NR^{d}R^{e},
c) —C(═NR^{c})—NR^{d}R^{e},
d) —NR^{f}—C(═NR^{c})—NR^{d}R^{e},
e) —NR^{f}—C(═NR^{c})—R^{c},
f) —NR^{f}—NR^{f}—C(═NR^{c})—NR^{d}R^{e},
g) —C(H)═N—NR^{f}—C(═NR^{c})—NR^{d}R^{e},
h) —C(═O)—NR^{f}—C(═NR^{c})—NR^{d}R^{e},
i) —C(═O)—NR^{f}—C(═NR^{c})—R^{c},
j) —(C₁-C₆)alkyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, and R^{j},
k) —(C₂-C₆)alkoxy that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, and or
l) phenyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, R^{j}, and R^{k}

each R^{b} is independently H, (C₁-C₆)alkyl, (C₁-C₆)alkoxy, (C₁-C₆)alkoxycarbonyl, nitro, halo, heteroaryl, —C(═O)NR^{v}R^{w}, —(OCH₂CH₂)_{y}—OR^{x}, —NR^{f}—SO₂—R^{c}, —NR^{f}—C(═O)—R^{c}, R^{m}, or Rⁿ;

each R^{c} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl;

each R^{d} and R^{e} is independently selected from H, (C₁-C₆)alkyl, (C₁-C₆)alkanoyl, (Cᵣ C₆)alkoxycarbonyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{d} and R^{e} together with the nitrogen to which they are attached form a aziridino, azetidino, morpholino, piperazino, pyrrolidino or piperidino; wherein any (C₁-C₆)alkyl, (C₃-C₆)cycloalkyl, (C₃-C₆)cycloalkyl(C₁-C₆)alkyl, aryl, heteroaryl, aryl(C₁-C₆)alkyl or heteroaryl(C₁-C₆)alkyl of R^{d} and R^{e} is optionally substituted with one or more groups independently selected from hydroxy, carboxy, and NR^{t}R^{u};

each R^{f} is H or (C₁-C₆)alkyl;

each R^{g} and R^{h} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{g} and R^{h} together with the nitrogen to which they are attached form a aziridino, azetidino, morpholino, piperazino, pyrrolidino or piperidino;

each R^{j} is independently selected from imidazoyl, piperazinyl, triazole, and piperazinyl that is optionally substituted with (C₁-C₆)alkyl;

each R^{k} is independently selected from —(C₁-C₆)alkyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e};

each R^{m} is independently selected from phenyl that is optionally substituted with one or more (C₁-C₆)alkyl, (C₁-C₆)alkoxy, trifluoromethyl, trifluoromethoxy, or halo;

each Rⁿ is independently selected from —(C₁-C₆)alkyl that is substituted with one or more groups independently selected from halo, hydroxy, and —NR^{g}R^{h};

each R^{t} and R^{u} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{t} and R^{u} together with the nitrogen to which they are attached form a aziridino, azetidino, morpholino, piperazino, pyrrolidino or piperidino;

each R^{v} and R^{w} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl, wherein any (C₁-C₆)alkyl of R^{v} and R^{w} is optionally substituted with one or more NR^{aa}R^{ab}; or R^{v} and R^{w} together with the nitrogen to which they are attached form a aziridino, azetidino, morpholino, piperazino, pyrrolidino or piperidino;

each R^{x} is H or (C₁-C₆)alkyl;

each R^{aa} and R^{ab} is H or (C₁-C₆)alkyl;

each R^{ac} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl;

X is a direct bond or —C≡C—;

y is 1, 2, 3, 4, 5, or 6; and

each Z⁻ is independently a suitable counterion;

or a pharmaceutically acceptable salt thereof.

The invention also provides a novel compound of formula I or a salt thereof.

The invention also provides a composition comprising a compound of formula I, or a pharmaceutically acceptable salt thereof, and a pharmaceutically acceptable vehicle.

The invention also provides a compound of formula I or a pharmaceutically acceptable salt thereof for the prophylactic or therapeutic treatment of a bacterial infection.

The invention also provides a compound of formula I or a pharmaceutically acceptable salt thereof for use in medical treatment.

The invention also provides the use of a compound of formula I or a pharmaceutically acceptable salt thereof for the preparation of a medicament for treating a bacterial infection in a mammal.

The invention also provides processes and intermediates disclosed herein that are useful for preparing compounds of formula I or salts thereof.

### DETAILED DESCRIPTION

The following definitions are used, unless otherwise described: halo is fluoro, chloro, bromo, or iodo. Alkyl, alkoxy, etc. denote both straight and branched groups but reference to an individual radical such as propyl embraces only the straight chain radical (a branched chain isomer such as isopropyl being specifically referred to). In one embodiment alkyl is a (C₁-C₆)alkyl and alkoxy is a (C₁-C₆)alkoxy. Aryl denotes a phenyl radical or an ortho-fused bicyclic carbocyclic radical having about nine to ten ring atoms in which at least one ring is aromatic. Heteroaryl encompasses a radical of a monocyclic aromatic ring containing five or six ring atoms consisting of carbon and one to four heteroatoms each selected from the group consisting of non-peroxide oxygen, sulfur, and N(Q) wherein Q is absent or is H, O, (C₁-C₄)alkyl, phenyl or benzyl; as well as a radical of an ortho-fused bicyclic heterocycle of about eight to ten ring atoms comprising one to four heteroatoms each selected from the group consisting of non-peroxide oxygen, sulfur, and N(Q).

As used herein “cycloalkyl” refers to a saturated or partially unsaturated cyclic hydrocarbon ring system. In one embodiment “cycloalkyl” includes (C₃-C₆)cycloalkyl which can be cyclopropyl, cyclobutyl, cyclopentyl, or cyclohexyl.

It will be appreciated by those skilled in the art that compounds of the invention having a chiral center may exist in and be isolated in optically active and racemic forms. Some compounds may exhibit polymorphism. It is to be understood that the present invention encompasses any racemic, optically-active, stereoisomeric, or polymorphic form, or mixtures thereof, of a compound of the invention, which possess the useful properties described herein, it being well known in the art how to prepare optically active forms (for example, by resolution of the racemic form by recrystallization techniques, by synthesis from optically-active starting materials, by chiral synthesis, or by chromatographic separation using a chiral stationary phase).

It will also be appreciated by those skilled in the art that certain compounds of the invention can exist in more than one tautomeric form. For example, a substituent of formula —NH—C(═NH)—NH₂ in a compound of formula (I) could exist in tautomeric form as —N═C(NH₂)—NH₂, or a substituent of formula —NH—C(═NH)—CH₃ in a compound of formula (I) could exist in tautomeric form as —N═C(NH₂)—CH₃. The present invention encompasses all tautomeric forms of a compound of formula I as well as mixtures thereof that can exist in equilibrium with non-charged entities depending upon pH, which possess the useful properties described herein.

Specific values listed below for radicals, substituents, and ranges, are for illustration only; they do not exclude other defined values or other values within defined ranges for the radicals and substituents.

Specifically, (C₁-C₆)alkyl can be methyl, ethyl, propyl, isopropyl, butyl, iso-butyl, sec-butyl, pentyl, 3-pentyl, or hexyl; (C₃-C₆)cycloalkyl can be cyclopropyl, cyclobutyl, cyclopentyl, or cyclohexyl; (C₁-C₆)alkoxy can be methoxy, ethoxy, propoxy, isopropoxy, butoxy, iso butoxy, sec-butoxy, pentoxy, 3-pentoxy, or hexyloxy; aryl can be phenyl, indenyl, or naphthoyl; and heteroaryl can be furyl, imidazolyl, triazolyl, triazinyl, oxazoyl, isoxazoyl, thiazolyl, isothiazoyl, pyrazolyl, pyrrolyl, pyrazinyl, tetrazolyl, pyridyl, (or its N-oxide), thienyl, pyrimidinyl (or its N-oxide), indolyl, benzimidazole, isoquinolyl (or its N-oxide) or quinolyl (or its N-oxide).

As used herein the term “aryl(C₁-C₆)alkyl” refers to a (C₁-C₆)alkyl radical in which one or more of the hydrogen atoms of the (C₁-C₆)alkyl radical is replaced with an aryl radical. As used herein the term “heteroaryl(C₁-C₆)alkyl” refers to a (C₁-C₆)alkyl radical in which one or more of the hydrogen atoms of the (C₁-C₆)alkyl radical is replaced with a heteroaryl radical. As used herein the term “(C₃-C₆)cycloalkylC₁-C₆)alkyl” refers to a (C₁-C₆)alkyl radical in which one or more of the hydrogen atoms of the (C₁-C₆)alkyl radical is replaced with a (C₃-C₆)cycloalkylradical.

In one embodiment of the invention ring A is phenyl, which is substituted with one or more R¹ and which is optionally substituted with one or more R^{b}.

In one embodiment of the invention ring A is thiazolyl, which is substituted with one or more R¹ and which is optionally substituted with one or more R^{b}.

In one embodiment of the invention R¹ is methyl, phenyl, tert-butyl, bromo, cyclohexyl, thiazolyl, biphenyl, thiazol-2-ylaminocarbonyl, or cyclopropyl.

In one embodiment of the invention B is phenyl substituted with one or more R^{a} and optionally substituted with one or more R^{b}.

In one embodiment of the invention B is pyridyl substituted with one or more R^{a} and optionally substituted with one or more R^{b}.

In one embodiment of the invention B is thiazolyl substituted with one or more R^{a} and optionally substituted with one or more R^{b}.

In one embodiment of the invention R^{a} is —N⁺(CH₃)₃Z⁻, —CH₂—N═C(NH₂)₂, —C(H)═N—NH—C(═NH)—NH₂, —C(CH₃)H—NH—C(═NH)—NH₂, 2-(1H-imidazol-1-yl)ethoxy, 1H-imidazol-1-ylmethyl, —O—CH₂—CH₂—NH—C(═NH)—NH₂, —CH₂—CH₂—N═C(NH₂)₂, —CH₂—CH₂—N⁺H₃Z⁻, 4-methylpiperazin-1-ylmethyl, aminomethyl, dimethylaminomethyl, 2-aminoethyl, N-but-2-ylaminomethyl, morpholinomethyl, 1-amino-1-methylethyl, —CH₂—NH—C(═NH)—NH—C(═O)OC(CH₃)₃, —CH₂—NH—NH—C(═NH)—NH₂, 1H-1,2,4-triazol-1-ylmethyl, —CH₂—N(CH(CH₃)CH₂CH₃)—C(═NH)—NH₂, —C(═O)—NH—C(═NH)—NH₂

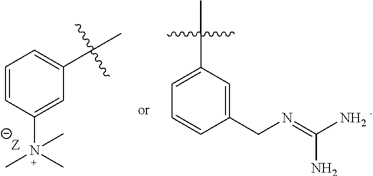

In one embodiment of the invention each R^{b} is selected from methoxy, methyl, N,N-dimethylaminomethyl, bromo, 4-tert-butylphenyl, 4-trifluoromethoxy-2-methoxyphenyl, nitro, amino, methylsulfonylamino, methylcarbonylamino, hydroxymethyl, 2-(N,N-diethylamino)ethylaminocarbonyl, methoxy, —(OCH₂CH₂)₄—OCH₃, 2,2-dibromoethyl, thiazol-2-ylaminocarbonyl, and methoxycarbonyl.

In one embodiment of the invention each R^{b} is selected from methoxy, N,N-dimethylaminomethyl, bromo, 4-tert-butylphenyl, 4-trifluoromethoxy-2-methoxyphenyl, nitro, and methoxycarbonyl.

In one embodiment of the invention R¹ is halo, —C(═O)NR^{v}R^{w}, phenyl, (C₁-C₆)alkyl, thiazolyl, R^{m}, or (C₃-C₆)cycloalkyl, which phenyl, (C₁-C₆)alkyl, thiazolyl, or (C₃-C₆)cycloalkyl is optionally substituted with one or more thiazolyl or —C(═O)NR^{v}R^{w}.

In one embodiment of the invention A is selected from:

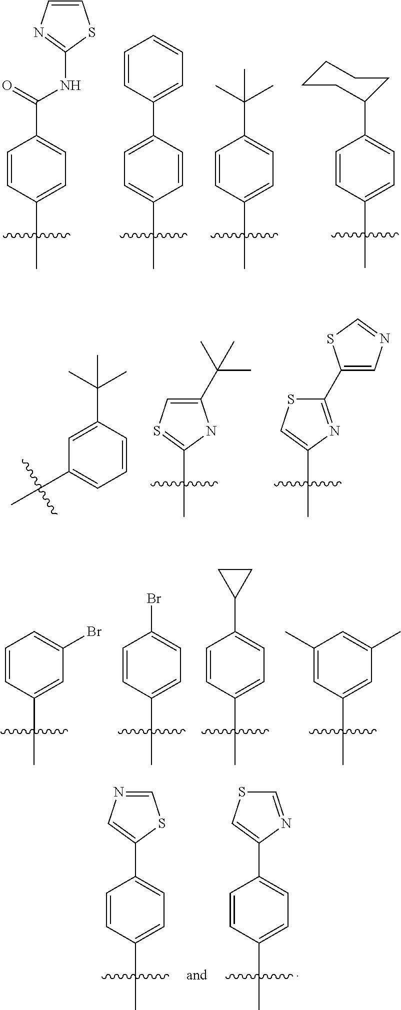

In one embodiment of the invention the compound of formula I is selected from:

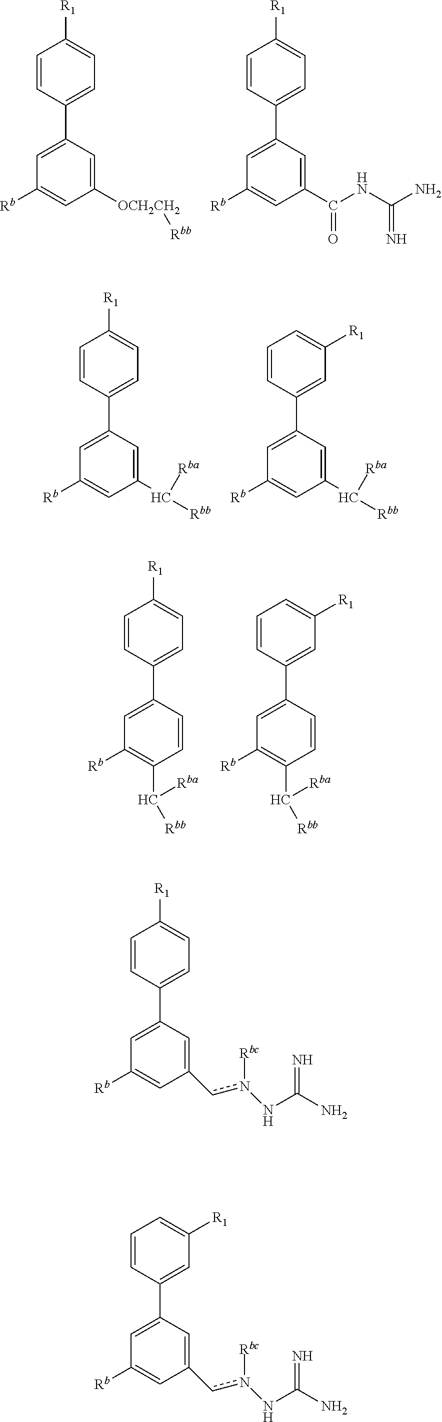

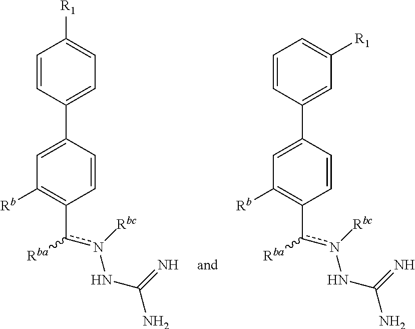

wherein:

the bond represented by --- is absent or is present to form a double bond.

each R^{ba} is H or methyl;

each R^{bb} is —NH—C(═NH)NH₂, —C(═NH)NH₂, or —CH₂NH₂; and

each ^{bc} is H or methyl, or is absent when the bond represented by --- is present.

In one embodiment of the invention each R¹ is independently selected from cyclopropyl, tert-butyl, bromo, 4-tert-butylphenyl, and phenyl that is substituted at the 4-position with halo.

In one embodiment of the invention each R^{b} is independently selected from H, chloro, bromo, fluoro, —NHSO₂H, and methoxy.

In one embodiment of the invention the compound of formula I is selected from:

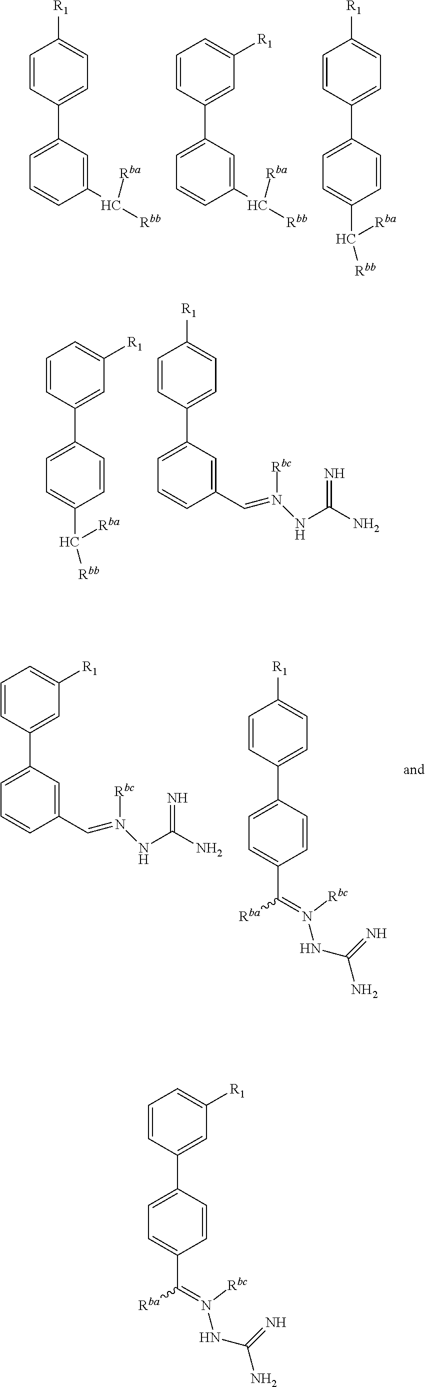

wherein:

the bond represented by --- is absent or is present to form a double bond.

each R^{ba} is H or methyl;

each R^{bb} is —NH—C(═NH)NH₂, —C(═NH)NH₂, or —CH₂NH₂; and

each ^{bc} is H or methyl, or is absent when the bond represented by --- is present.

In one embodiment the invention provides a compound selected from compounds of formula I and salts thereof having a minimal inhibitory concentration against MSSA of less than about 16 μm (see Test C below).

In one embodiment the invention provides a compound selected from compounds of formula I and salts thereof having a minimal inhibitory concentration against MSSA of less than about 8 μm.

In one embodiment the invention provides a compound selected from compounds of formula I and salts thereof having a minimal inhibitory concentration against MSSA of less than about 4 μm.

In one embodiment the invention provides a compound selected from compounds of formula I and salts thereof having a minimal inhibitory concentration against MSSA of less than about 2 μm.

In one embodiment the invention provides a compound selected from compounds of formula I and salts thereof having a minimal inhibitory concentration against MRSA of less than about 16 μm (see Test C below).

In one embodiment the invention provides a compound selected from compounds of formula I and salts thereof having a minimal inhibitory concentration against MRSA of less than about 8 μm.

In one embodiment the invention provides a compound selected from compounds of formula I and salts thereof having a minimal inhibitory concentration against MRSA of less than about 4 μm.

In one embodiment the invention provides a compound selected from compounds of formula I and salts thereof having a minimal inhibitory concentration against MRSA of less than about 2 μm.

Generally, compounds of I as well as synthetic intermediates that can be used for preparing compounds of formula I, can be prepared as illustrated in the following Schemes. It is understood that variable groups (e.g. A, R, R₁, Het, etc.) shown in the Schemes below can represent the final corresponding groups present in a compound of formula I or that these groups can represent groups that can be converted to the final corresponding groups in a compound of formula I at a convenient point in a synthetic sequence. For example, in the Schemes below, the variable groups can contain one or more protecting groups that can be removed at a convenient point in a synthetic sequence to provide the final corresponding groups in the compound of formula I. Processes for preparing compounds of formula I are provided as further embodiments of the invention and are illustrated by the following procedures.

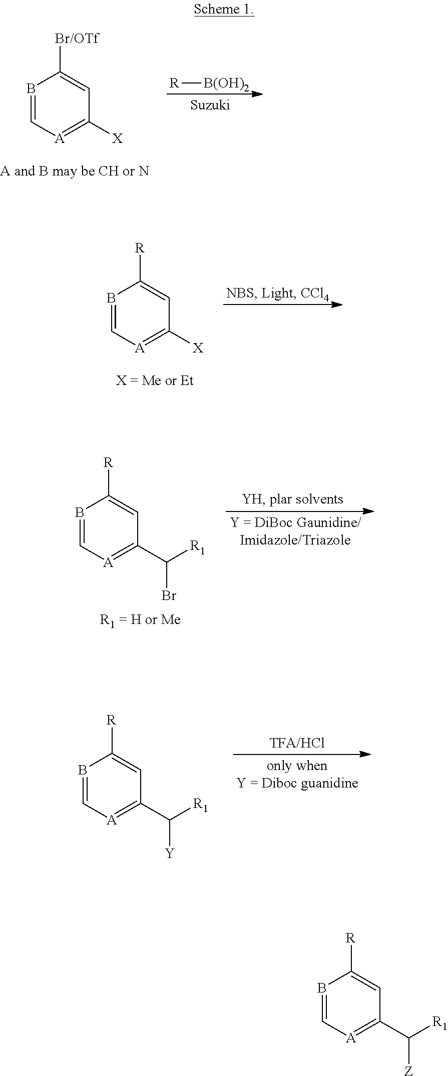

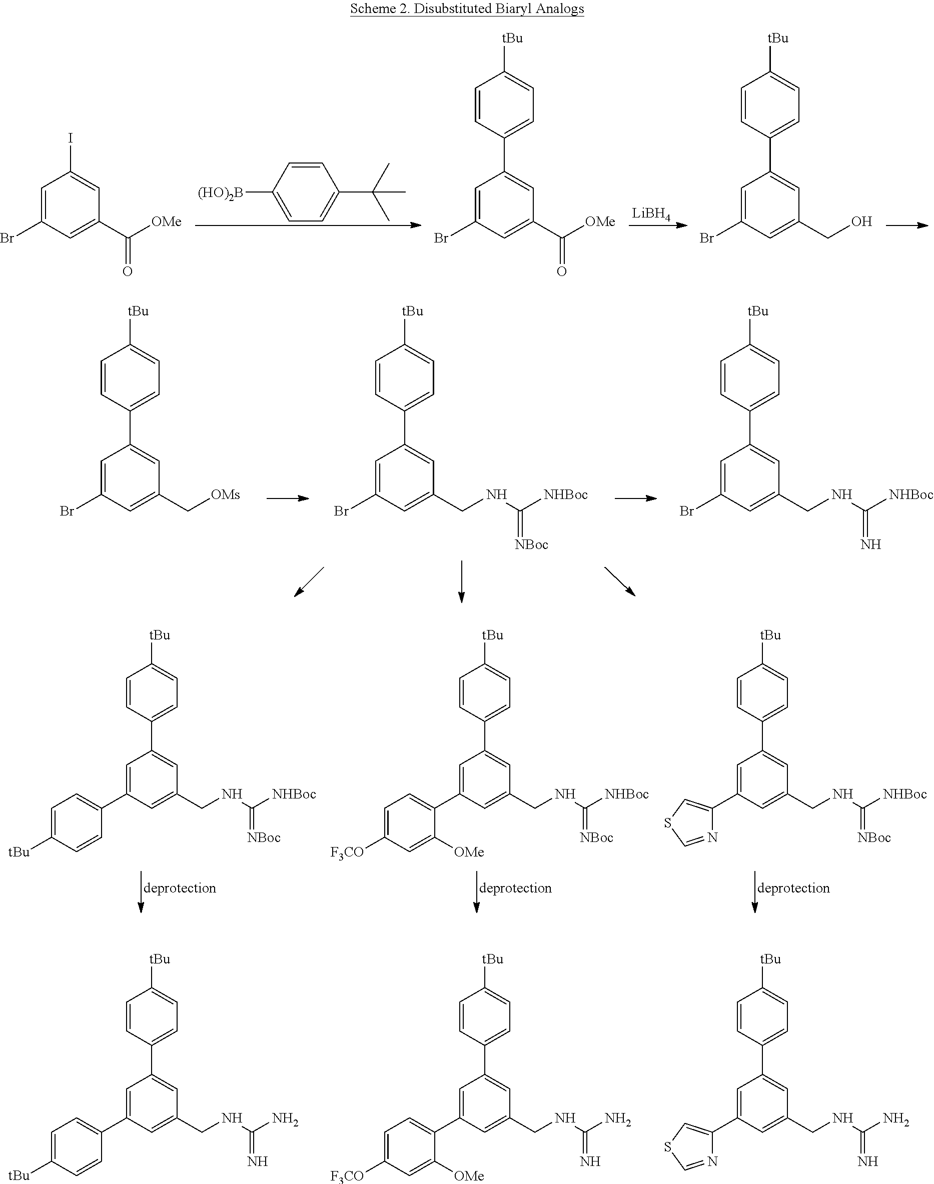

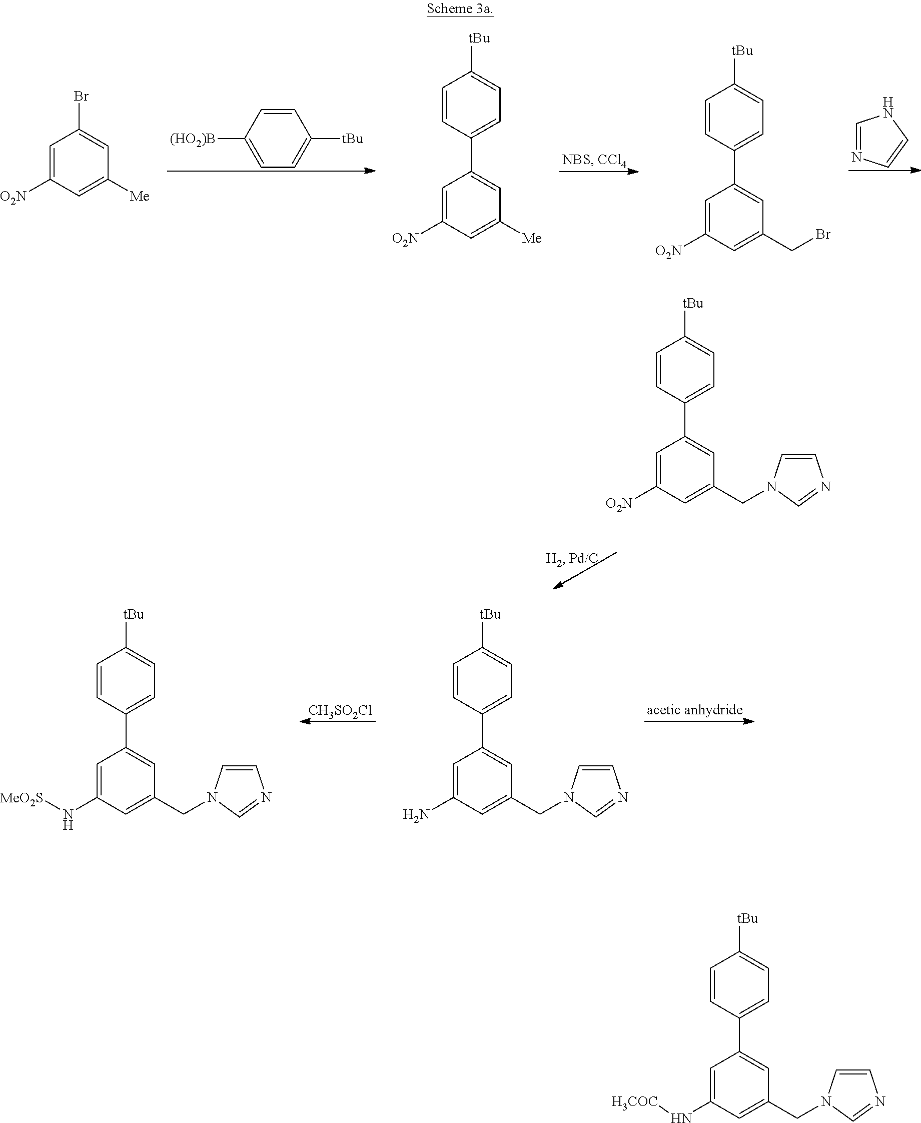

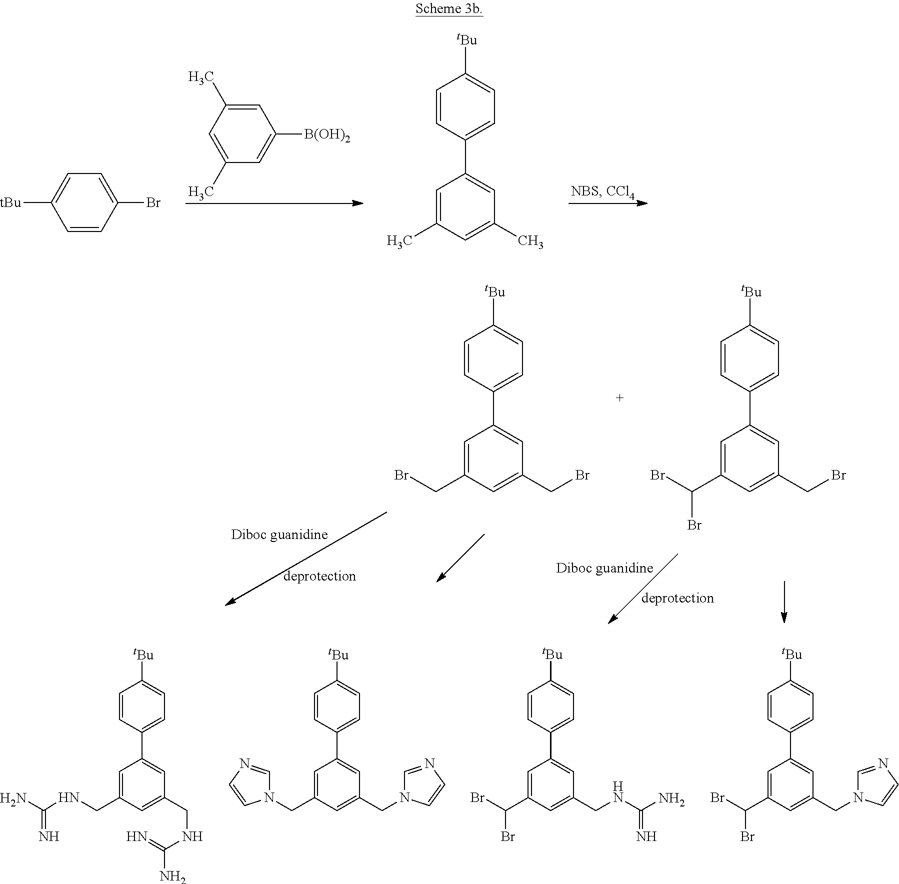

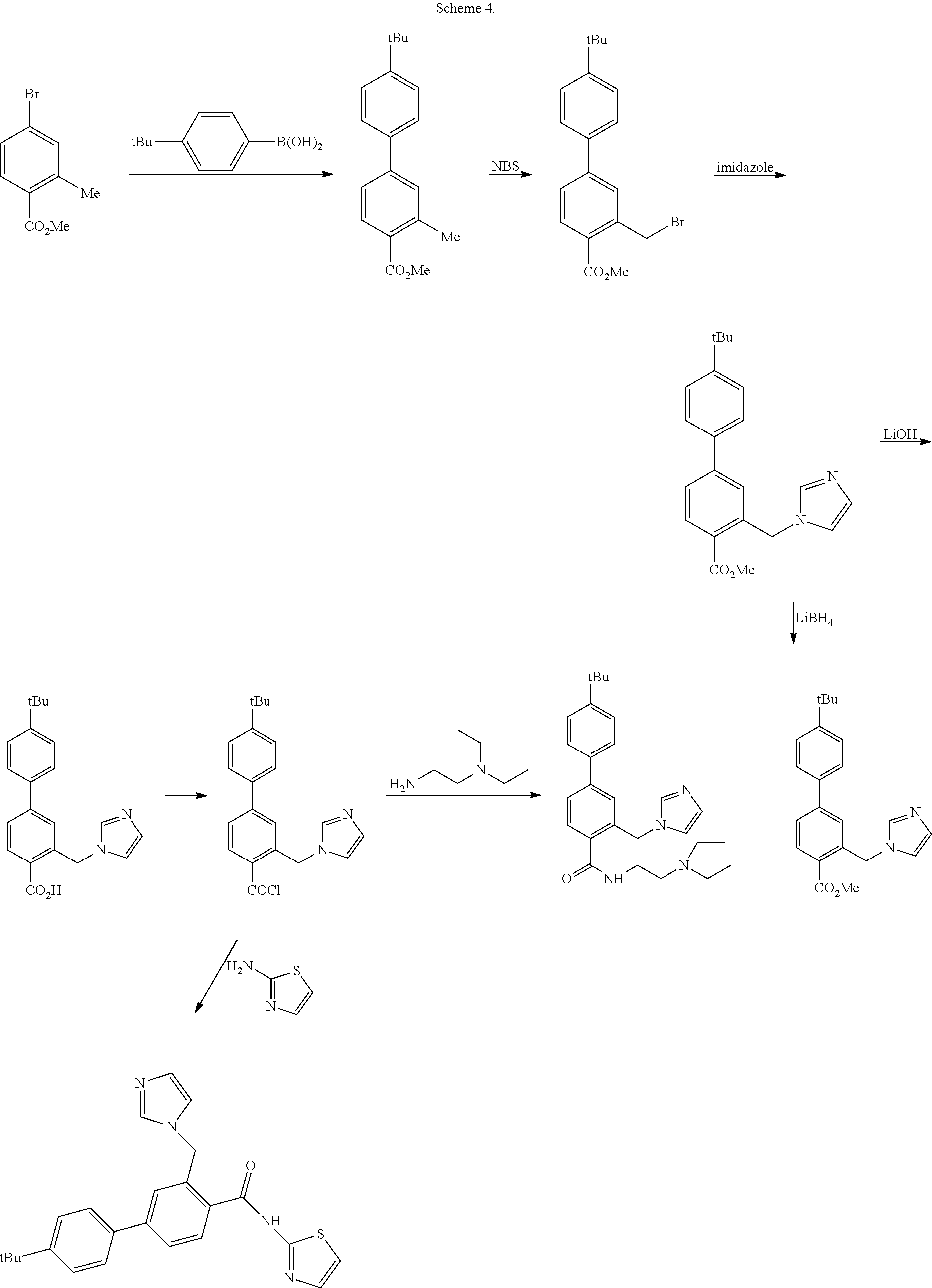

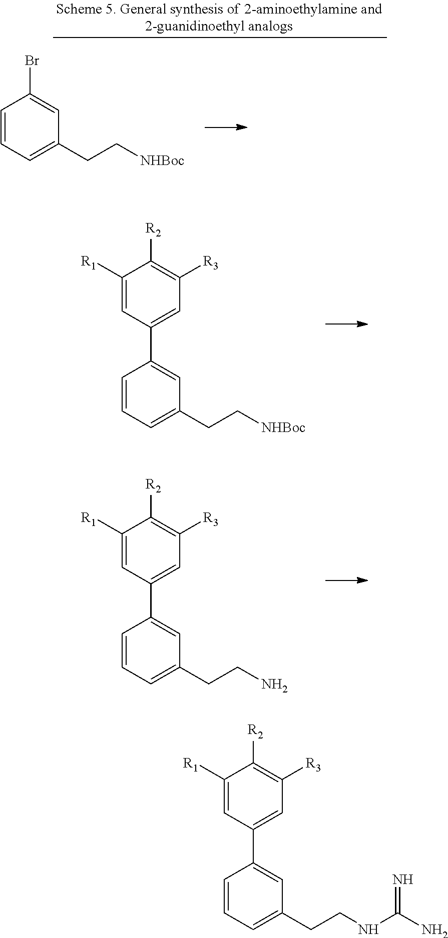

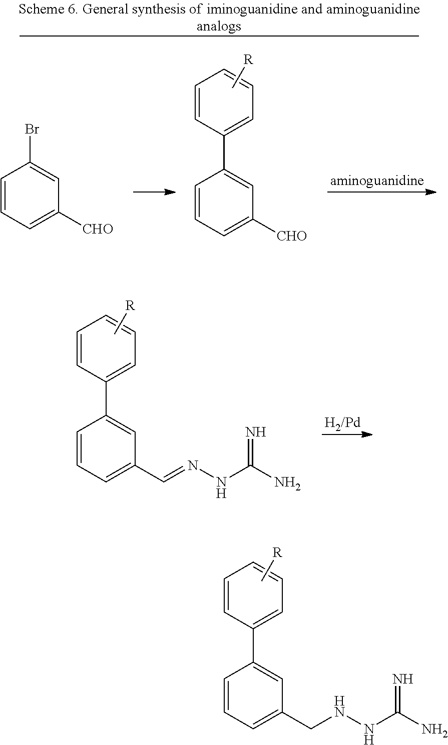

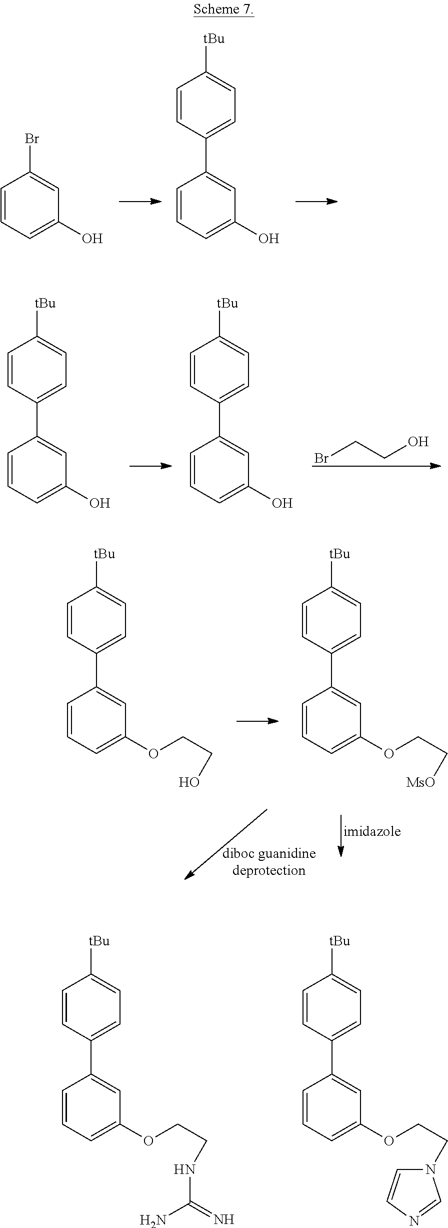

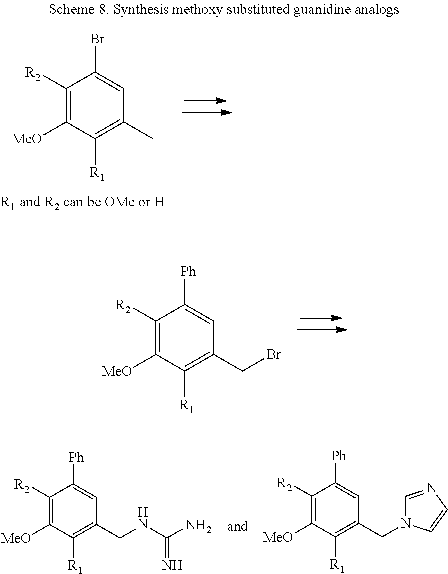

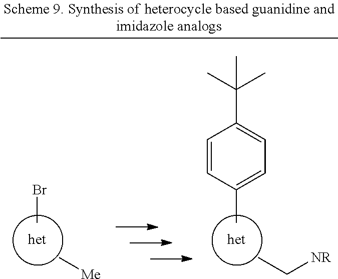

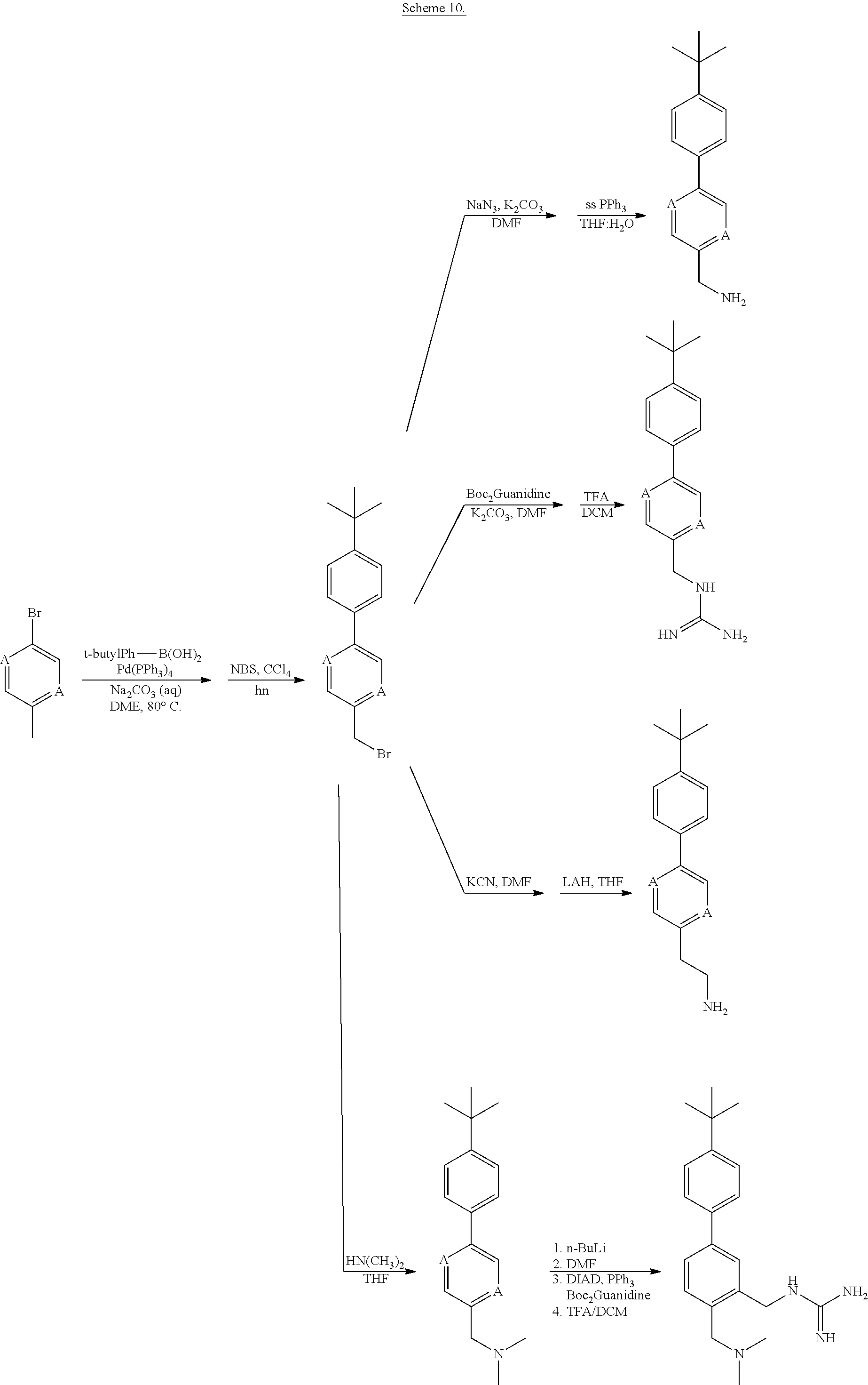

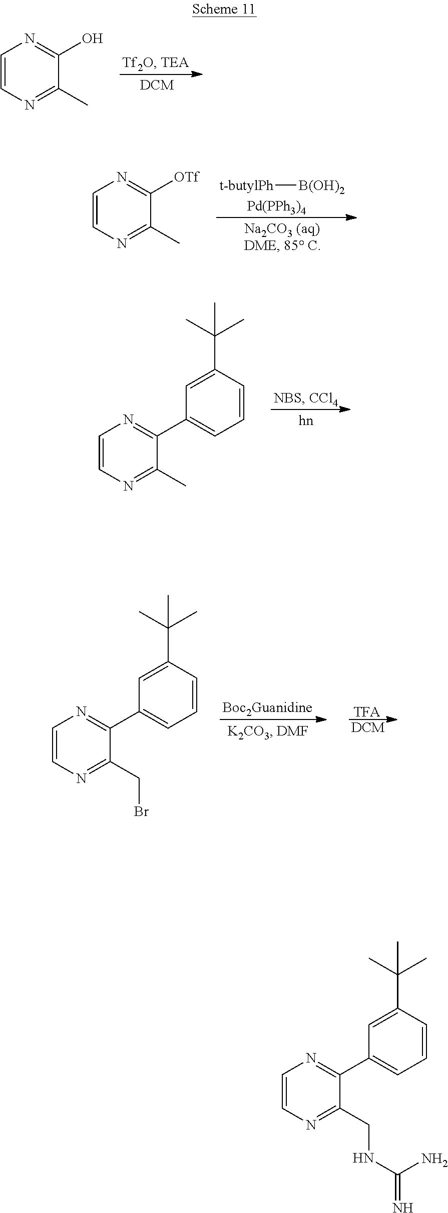

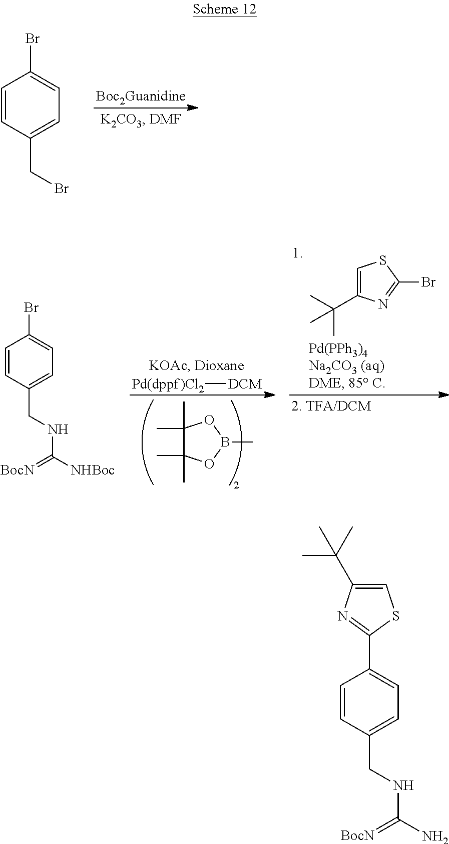

By binding to FtsZ, the compounds of the present invention inhibit the ability of the protein to hydrolyze GTP. This inhibition of FtsZ GTPase activity, in turn, inhibits the ability of the protein to polymerize into Z-rings, as Z-ring formation requires GTP hydrolysis as an energy source for driving the reaction. Since the Z-ring serves as the scaffold for recruitment of all other proteins that comprise the divisome complex, inhibition of Z-ring formation by the compounds of the present invention also results in a corresponding inhibition of divisome protein recruitment.

The compounds of the invention are useful to treat bacterial infections including infections by Gram-negative bacterial strains, Gram-positive bacterial strains and multiple drug-resistant bacterial strains

Gram-negative bacterial strains include Escherchia coli, Caulobacter crescentus, Pseudomonas aeruginosa, Agrobacterium tumefaciens, Branhamella catarrhalis, Citrobacter diversus, Enterobacter aerogenes, Enterobacter cloacae, Enterobacter sakazakii, Enterobacter asburiae, Pantoea agglomerans, Klebsiella pneumoniae, Klebsiella oxytoca, Klebsiella rhinoscleromatis, Proteus mirabilis, Salmonella typhimurium, Salmonella enteriditis, Serratia marcescens, Shigella sonnei, Neisseria gonorrhoeae, Acinetobacter baumannii, Acinetobacter calcoaceticus, Acinetobacter lwoffi, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Borrelia burgdorferi, Neisseria meningitidis and Haemophilus influenza.

Gram-positive bacterial strains include Staphylococcus aureus, Staphylococcus epidermidis, Staphylococcus saprophyticus, Streptococcus pyogenes, Streptococcus faecalis, Enterococcus faecalis, Enterococcus faecium, Bacillus subtilis, Bacillus anthracis, Bacillus cereus, Micrococcus luteus, Mycobacterium tuberculosis, Clostridium difficile, Propionibacterium acnes, Streptococcus mutans, Actinomyces viscosus, Actinomyces naeslundii, Streptococcus sanguis, Streptococcus pneumoniae, Streptococcus viridans and Streptococcus salivarius.

Multiple drug-resistant bacterial strains include methicillin-resistant Staphylococcus aureus, vancomycin-resistant Enterococci, multiple drug-resistant Mycobacterium tuberculosis, and multidrug-resistant Clostridium difficile.

In one embodiment compounds of the present invention may be administered as a composition used to treat and/or prevent a bacterial infection wherein the bacterial cell uses polymerized FtsZ protein, or a homolog thereof, to facilitate cytokinesis. To this end, compounds of the present invention may be administered to treat Staph Infections, Tuberculosis, Urinary Tract Infections, Meningitis, Enteric Infections, Wound Infections, Acne, Encephalitis, Skin Ulcers, Bed Sores, Gastric and Duodenal Ulcers, Eczema, Periodontal disease, Gingivitis, Halitosis, Anthrax, Tularemia, Endocarditis, Prostatitis, Osteomyelitis, Lyme Disease, Pneumonia, or the like.

The compositions can, if desired, also contain other active therapeutic agents, such as a narcotic, a non-steroid anti-inflammatory drug (NSAID), an analgesic, an anesthetic, a sedative, a local anesthetic, a neuromuscular blocker, an anti-cancer, other antimicrobial (for example, an aminoglycoside, an antifungal, an antiparasitic, an antiviral, a carbapenem, a cephalosporin, a flurorquinolone, a macrolide, a penicillin, a sulfonamide, a tetracycline, another antimicrobial), an anti-psoriatic, a corticosteriod, an anabolic steroid, a diabetes-related agent, a mineral, a nutritional, a thyroid agent, a vitamin, a calcium-related hormone, an antidiarrheal, an anti-tussive, an anti-emetic, an anti-ulcer, a laxative, an anticoagulant, an erythropieitin (for example, epoetin alpha), a filgrastim (for example, G-CSF, Neupogen), a sargramostim (GM-CSF, Leukine), an immunization, an immunoglobulin, an immunosuppressive (for example, basiliximab, cyclosporine, daclizumab), a growth hormone, a hormone replacement drug, an estrogen receptor modulator, a mydriatic, a cycloplegic, an alkylating agent, an anti-metabolite, a mitotic inhibitor, a radiopharmaceutical, an anti-depressant, an anti-manic agent, an anti-psychotic, an anxiolytic, a hypnotic, a sympathomimetic, a stimulant, donepezil, tacrine, an asthma medication, a beta agonist, an inhaled steroid, a leukotriene inhibitor, a methylxanthine, a cromolyn, an epinephrine or analog thereof, dornase alpha (Pulmozyme), a cytokine, or any combination thereof.

The term “prodrug” as used herein refers to any compound that when administered to a biological system (e.g. a mammal such as a human) generates the drug substance, i.e. active ingredient, as a result of spontaneous chemical reaction(s), enzyme catalyzed chemical reaction(s), photolysis, and/or metabolic chemical reaction(s) or by some other process. A prodrug is thus a modified (e.g. covalently modified) analog or latent form of a therapeutically-active compound. A prodrug may also be an active metabolite or therapeutically-active compound itself.

By way of example a prodrug may generate the active inhibitory compound during metabolism, systemically, inside a cell, by hydrolysis, enzymatic cleavage, or by some other process (Bundgaard, Hans, “Design and Application of Prodrugs” in A Textbook of Drug Design and Development (1991), P. Krogsgaard-Larsen and H. Bundgaard, Eds. Harwood Academic Publishers, pp. 113-191; Tranoyl-Opalinski, I., Fernandes, A., Thomas, M., Gesson, J.-P., and Papot, S., Anti-Cancer Agents in Med. Chem., 8 (2008) 618-637). Enzymes which are capable of an enzymatic activation mechanism with the prodrug compounds of the invention include, but are not limited to nitroreductase, proteases (e.g. serine proteases such as prostate specific antigen (PSA), amidases, esterases, microbial enzymes, phospholipases, cholinesterases, and phosphases).

In cases where compounds are sufficiently basic or acidic, a salt of a compound of formula I can be useful as an intermediate for isolating or purifying a compound of formula I. Additionally, administration of a compound of formula I as a pharmaceutically acceptable acid or base salt may be appropriate. Examples of pharmaceutically acceptable salts are organic acid addition salts formed with acids which form a physiological acceptable anion, for example, tosylate, methanesulfonate, acetate, citrate, malonate, tartrate, succinate, benzoate, ascorbate, α-ketoglutarate, and α-glycerophosphate. Suitable inorganic salts may also be formed, including hydrochloride, sulfate, nitrate, bicarbonate, and carbonate salts. Salts may be obtained using standard procedures well known in the art, for example by reacting a sufficiently basic compound such as an amine with a suitable acid affording the corresponding anion. Alkali metal (for example, sodium, potassium or lithium) or alkaline earth metal (for example calcium) salts of carboxylic acids can also be made.

Pharmaceutically suitable counterions include pharmaceutically suitable cations and pharmaceutically suitable anions that are well known in the art. Examples of pharmaceutically suitable anions include, but are not limited to those described above (e.g. physiologically acceptable anions) including Cl⁻, F⁻, CH₃SO₃⁻, CF₃SO₃⁻, p-CH₃C₆H₄SO₃⁻, citrate, tartrate, malate, fumarate, formate, or acetate.

It will be appreciated by those skilled in the art that a compound of the invention comprising a counterion can be converted to a compound of the invention comprising a different counterion. Such a conversion can be accomplished using a variety of well known techniques and materials including but not limited to ion exchange resins, ion exchange chromatography and selective crystallization.

The compounds of formula I can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

Thus, the present compounds may be systemically administered, e.g., orally, in combination with a pharmaceutically acceptable vehicle such as an inert diluent, excipient or an assimilable edible carrier. They may be enclosed in hard or soft shell gelatin capsules, may be compressed into tablets, or may be incorporated directly with the food of the patient's diet. For oral therapeutic administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 90% of the weight of a given unit dosage form. The amount of active compound in such therapeutically useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations, particles, and devices.

The active compound may also be administered intravenously or intramuscularly by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersion, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina, nanoparticles, and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of formula I can be determined by comparing their in vitro activity, and in vivo activity in animal models. Methods for the extrapolation of effective dosages in mice, and other animals, to humans are known to the art; for example, see U.S. Pat. No. 4,938,949.

The amount of the compound, or an active salt or derivative thereof, required for use in treatment will vary not only with the particular salt selected but also with the route of administration, the nature of the condition being treated and the age and condition of the patient and will be ultimately at the discretion of the attendant physician or clinician.

In general, however, a suitable dose will be in the range of from about 0.1 to about 500 mg/kg, e.g., from about 0.5 to about 400 mg/kg of body weight per day, such as 1 to about 250 mg per kilogram body weight of the recipient per day.

The compound is conveniently formulated in unit dosage form; for example, containing 0.5 to 500 mg, 1 to 400 mg, or 0.5 to 100 mg of active ingredient per unit dosage form. In one embodiment, the invention provides a composition comprising a compound of the invention formulated in such a unit dosage form.

The desired dose may conveniently be presented in a single dose or as divided doses administered at appropriate intervals, for example, as two, three, four or more sub-doses per day. The sub-dose itself may be further divided, e.g., into a number of discrete loosely spaced administrations.

The impact of a compound of the invention on the dynamics of bacterial FtsZ polymerization can be determined using a method like Test A described below.

### Test A. FtsZ Polymerization Assay.

Compound-induced alteration in the dynamics of FtsZ polymerization can be tested using a turbidity assay with purified FtsZ protein. Upon addition of GTP, FtsZ self-associates to form polymeric structures that scatter light at 340 nm to a greater extent than the monomeric protein. The impact of the compounds of the invention on the polymerization dynamics of FtsZ can be detected by an increase or decrease in the extent of GTP-induced light scattering (as determined by corresponding changes in optical density at 340 nm) relative to that observed in the absence of compound. Quantitation of the overall extent of light scattering as a function of compound concentration provides an indication of the potency of that compound at altering the dynamics of FtsZ polymerization.

The impact of a compound of the invention on FtsZ Z-ring formation in bacteria can be determined using a method like Test B described below.

### Test B. FtsZ Z-Ring Assay.

The impact of a compound on FtsZ Z-ring formation can be determined using a strain of Bacillus subtilis (FG347) that expresses a green fluorescent protein (GFP)-ZapA fusion protein upon induction with xylose. ZapA is known to associate with FtsZ Z-rings in B. subtilis and, thus, serves as a marker for Z-ring formation. In this assay, log-phase FG347 bacteria are treated with differing concentrations of compound for time periods ranging from 1 to 6 hours. At each time point, aliquots are taken from each culture and then viewed with a fluorescence microscope. In the absence of compound, the bacteria exhibit green fluorescent foci (Z-rings) localized at mid-cell. By contrast, bacteria treated with a compound that disrupts Z-ring formation do not exhibit the green fluorescent Z-rings at mid-cell and are typically associated with an elongated, filamentous phenotype.

The antibacterial activity of a compound of the invention can be determined using a method like Test C described below.

### Test C. Antibacterial Assay.

Antibacterial activity can be determined as per Clinical and Laboratory Standards Institute (CLSI) guidelines using a broth microdilution assay in which log-phase bacteria are grown at 37° C. in appropriate medium containing two-fold serial dilutions of a compound to yield final concentrations ranging from 256 to 0.06 μg/mL. For determination of minimal inhibitory concentration (MIC) values, bacterial growth is monitored after 24 to 48 hours by measuring optical density at 600 nm. MIC values reflect the minimal compound concentrations at which bacterial growth is completely inhibited.

Using a procedure similar to Test C, representative compounds of the invention were tested against methicillin-susceptible Staphylococcus aureus (MSSA) and methicillin-resistant Staphylococcus aureus (MRSA). Results are shown in Table 1.

TABLE 1
Minimal Inhibitory Concentrations against MSSA and MRSA for representative compounds of the Invention

| | | MSSA | MRSA | |
|---|---|---|---|---|
| | | MIC data | MIC data | |
| Code | Structure | (μg/mL) | (μg/mL)* | Example |
| 1 | 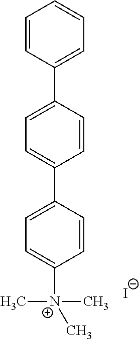 | 8.0 | 64 | 1 |
| 2 | 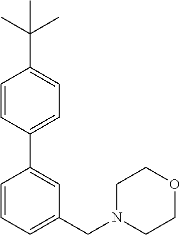 [SMILES: CC(C)(C)c1ccc(-c2cccc(CN3CCOCC3)c2)cc1] | >64.0 | n/d | |
| 3 | 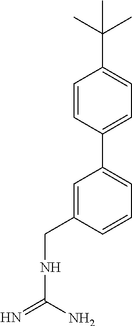 [SMILES: CC(C)(C)c1ccc(-c2cccc(CNC(=N)N)c2)cc1] | 1.0 | 1.0 | 2 |
| 4 | 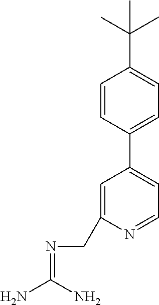 [SMILES: CC(C)(C)c1ccc(-c2ccnc(CN=C(N)N)c2)cc1] | 8.0 | 16 | 3 |
| 5 | 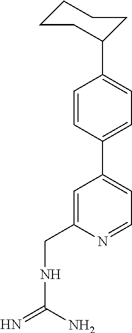 [SMILES: N=C(N)NCc1cc(-c2ccc(C3CCCCC3)cc2)ccn1] | 4.0 | 8.0 | 4 |
| 6 | 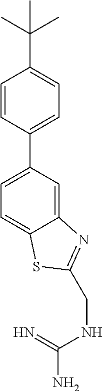 [SMILES: CC(C)(C)c1ccc(-c2ccc3sc(CNC(=N)N)nc3c2)cc1] | 4.0 | 8.0 | 5 |
| 7 | 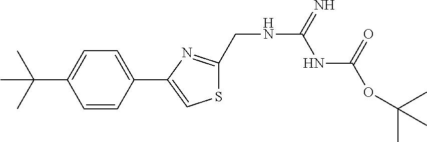 [SMILES: CC(C)(C)OC(=O)NC(=N)NCc1nc(-c2ccc(C(C)(C)C)cc2)cs1] | 8.0 | n/d | |
| 8 | 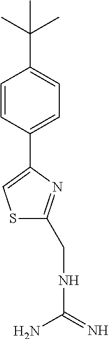 [SMILES: CC(C)(C)c1ccc(-c2csc(CNC(=N)N)n2)cc1] | 4.0 | 32 | 6 |
| 9 | 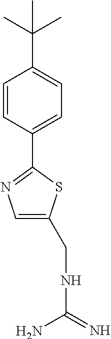 [SMILES: CC(C)(C)c1ccc(-c2ncc(CNC(=N)N)s2)cc1] | 8.0 | 32 | 7 |
| 10 | 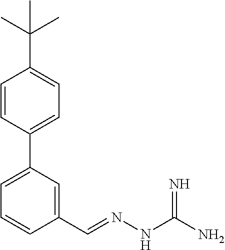 [SMILES: CC(C)(C)c1ccc(-c2cccc(/C=N/NC(=N)N)c2)cc1] | 2.0 | 8.0 | 8 |
| 11 | 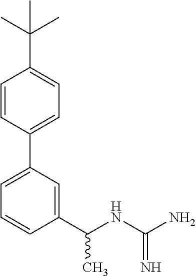 [SMILES: CC(NC(=N)N)c1cccc(-c2ccc(C(C)(C)C)cc2)c1] | 1.0 | 2.0 | 9 |
| 12 | 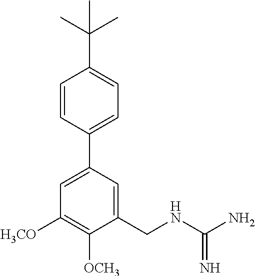 [SMILES: COc1cc(-c2ccc(C(C)(C)C)cc2)cc(CNC(=N)N)c1OC] | 2.0 | 8.0 | 10 |
| 13 | 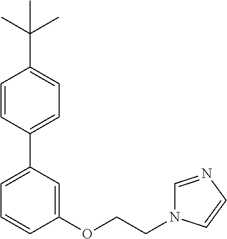 [SMILES: CC(C)(C)c1ccc(-c2cccc(OCCn3ccnc3)c2)cc1] | 4.0 | 4.0 | 11 |
| 14 | 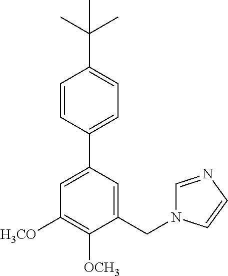 [SMILES: COc1cc(-c2ccc(C(C)(C)C)cc2)cc(Cn2ccnc2)c1OC] | 2.0 | 2.0 | 12 |
| 15 | 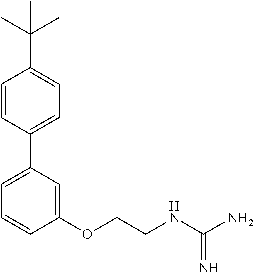 [SMILES: CC(C)(C)c1ccc(-c2cccc(OCCNC(=N)N)c2)cc1] | 2.0 | 4.0 | 13 |
| 16 | 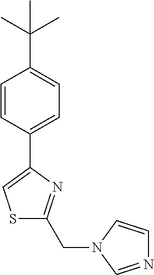 [SMILES: CC(C)(C)c1ccc(-c2csc(Cn3ccnc3)n2)cc1] | 4.0 | 16 | 14 |
| 17 | 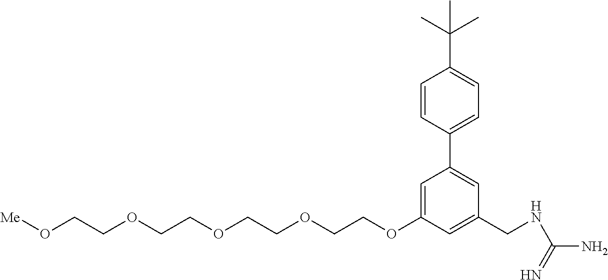 [SMILES: COCCOCCOCCOCCOc1cc(CNC(=N)N)cc(-c2ccc(C(C)(C)C)cc2)c1] | 4.0 | 4.0 | 15 |
| 18 | 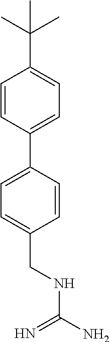 [SMILES: CC(C)(C)c1ccc(-c2ccc(CNC(=N)N)cc2)cc1] | 2.0 | 4.0 | 16 |
| 19 | 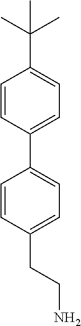 [SMILES: CC(C)(C)c1ccc(-c2ccc(CCN)cc2)cc1] | 8.0 | 16 | 17 |
| 20 | 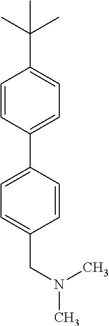 [SMILES: CN(C)Cc1ccc(-c2ccc(C(C)(C)C)cc2)cc1] | 16 | 32 | 18 |
| 21 | 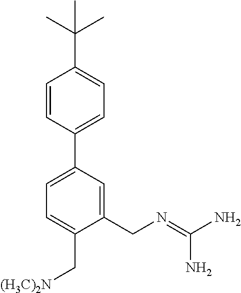 | 4.0 | 8.0 | 19 |
| 22 | 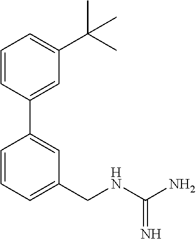 [SMILES: CC(C)(C)c1cccc(-c2cccc(CNC(=N)N)c2)c1] | 2.0 | 8.0 | 20 |
| 23 | 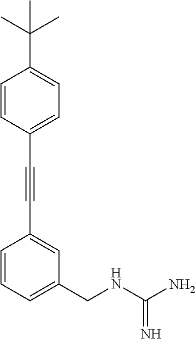 [SMILES: CC(C)(C)c1ccc(C#Cc2cccc(CNC(=N)N)c2)cc1] | 4.0 | 8.0 | 21 |
| 24 | 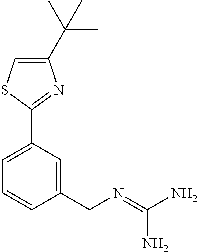 [SMILES: CC(C)(C)c1csc(-c2cccc(CN=C(N)N)c2)n1] | 4.0 | 16 | 22 |
| 25 | 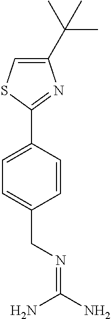 [SMILES: CC(C)(C)c1csc(-c2ccc(CN=C(N)N)cc2)n1] | 4.0 | 8 | 23 |
| 26 | 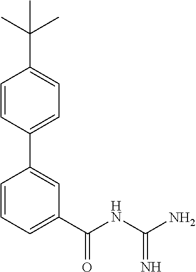 [SMILES: CC(C)(C)c1ccc(-c2cccc(C(=O)NC(=N)N)c2)cc1] | 4.0 | 8.0 | 24 |
| 27 | 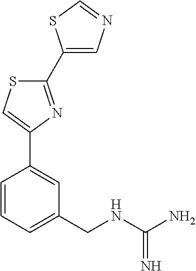 [SMILES: N=C(N)NCc1cccc(-c2csc(-c3cncs3)n2)c1] | 32 | n/d | |
| 28 | 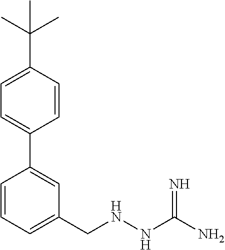 [SMILES: CC(C)(C)c1ccc(-c2cccc(CNNC(=N)N)c2)cc1] | 2.0 | 4.0 | 25 |
| 29 | 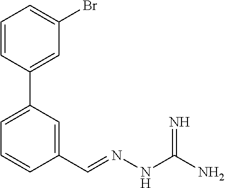 [SMILES: N=C(N)N/N=C/c1cccc(-c2cccc(Br)c2)c1] | 4.0 | 8.0 | 26 |
| 30 | 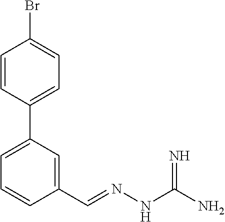 [SMILES: N=C(N)N/N=C/c1cccc(-c2ccc(Br)cc2)c1] | 4.0 | 4.0 | 27 |
| 31 | 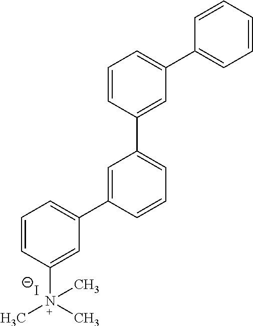 | 1.0 | 4.0 | 28 |
| 32 | 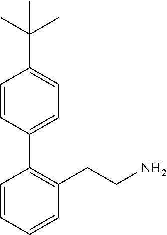 [SMILES: CC(C)(C)c1ccc(-c2ccccc2CCN)cc1] | 32 | n/d | |
| 33 | 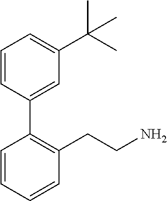 [SMILES: CC(C)(C)c1cccc(-c2ccccc2CCN)c1] | 32 | n/d | |
| 34 | 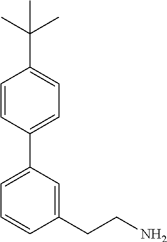 [SMILES: CC(C)(C)c1ccc(-c2cccc(CCN)c2)cc1] | 16 | 8.0 | 29 |
| 35 | 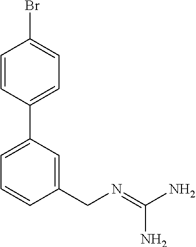 [SMILES: NC(N)=NCc1cccc(-c2ccc(Br)cc2)c1] | 4.0 | 8.0 | 30 |
| 36 | 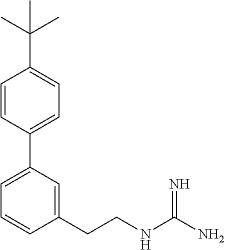 [SMILES: CC(C)(C)c1ccc(-c2cccc(CCNC(=N)N)c2)cc1] | 2.0 | 4.0 | 31 |
| 37 | 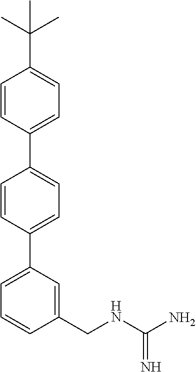 [SMILES: CC(C)(C)c1ccc(-c2ccc(-c3cccc(CNC(=N)N)c3)cc2)cc1] | 1.0 | 2.0 | 32 |
| 38 | 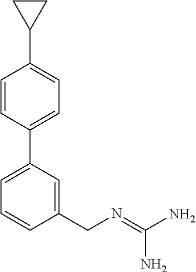 [SMILES: NC(N)=NCc1cccc(-c2ccc(C3CC3)cc2)c1] | 4.0 | 32 | 33 |
| 39 | 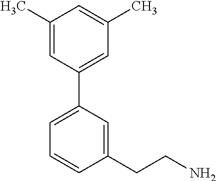 [SMILES: Cc1cc(C)cc(-c2cccc(CCN)c2)c1] | 64 | n/d | |
| 40 | 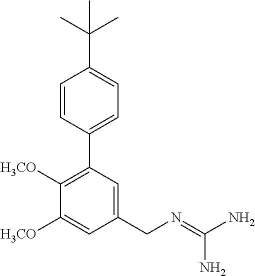 [SMILES: COc1cc(CN=C(N)N)cc(-c2ccc(C(C)(C)C)cc2)c1OC] | 4.0 | 16 | 34 |
| 41 | 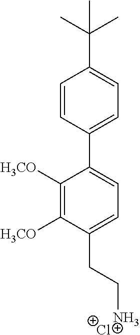 | 8.0 | 16 | 35 |
| 42 | 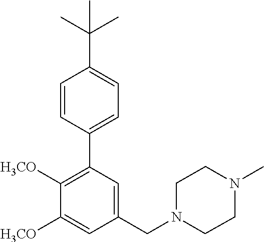 [SMILES: COc1cc(CN2CCN(C)CC2)cc(-c2ccc(C(C)(C)C)cc2)c1OC] | 8.0 | 8.0 | 36 |
| 43 | 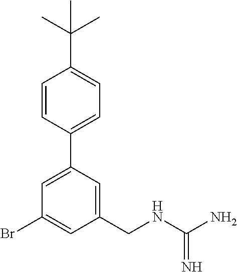 [SMILES: CC(C)(C)c1ccc(-c2cc(Br)cc(CNC(=N)N)c2)cc1] | 1.0 | 2.0 | 37 |
| 44 | 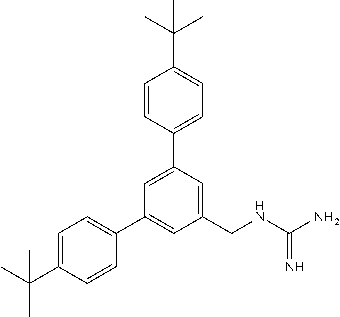 [SMILES: CC(C)(C)c1ccc(-c2cc(CNC(=N)N)cc(-c3ccc(C(C)(C)C)cc3)c2)cc1] | 1.0 | 2.0 | 38 |
| 45 | 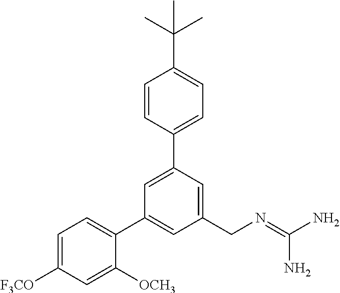 [SMILES: COc1cc(OC(F)(F)F)ccc1-c1cc(CN=C(N)N)cc(-c2ccc(C(C)(C)C)cc2)c1] | 1.0 | 2.0 | 39 |
| 46 | 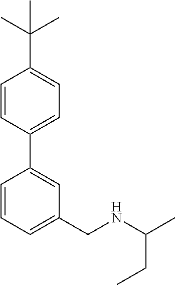 [SMILES: CCC(C)NCc1cccc(-c2ccc(C(C)(C)C)cc2)c1] | 16 | 32 | 40 |
| 47 | 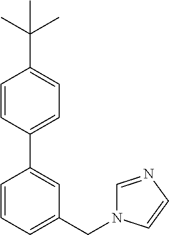 [SMILES: CC(C)(C)c1ccc(-c2cccc(Cn3ccnc3)c2)cc1] | 2.0 | 8.0 | 41 |
| 48 | 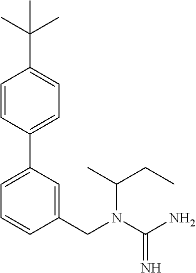 [SMILES: CCC(C)N(Cc1cccc(-c2ccc(C(C)(C)C)cc2)c1)C(=N)N] | >64 | n/d | |
| 49 | 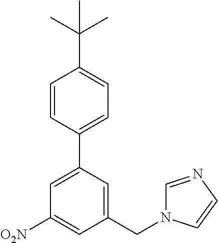 [SMILES: CC(C)(C)c1ccc(-c2cc(Cn3ccnc3)cc([N+](=O)[O-])c2)cc1] | 8.0 | 8.0 | 42 |
| 50 | 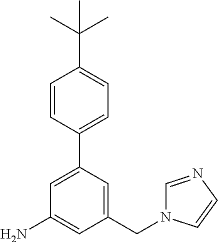 [SMILES: CC(C)(C)c1ccc(-c2cc(N)cc(Cn3ccnc3)c2)cc1] | >64.0 | n/d | |
| 51 | 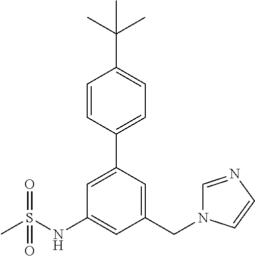 [SMILES: CC(C)(C)c1ccc(-c2cc(Cn3ccnc3)cc(NS(C)(=O)=O)c2)cc1] | 16.0 | >64 | 43 |
| 52 | 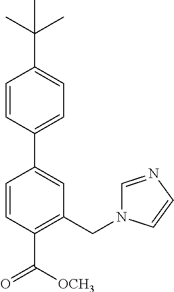 [SMILES: COC(=O)c1ccc(-c2ccc(C(C)(C)C)cc2)cc1Cn1ccnc1] | 2.0 | 8.0 | 44 |
| 53 | 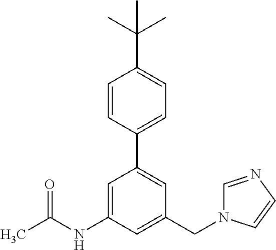 [SMILES: CC(=O)Nc1cc(Cn2ccnc2)cc(-c2ccc(C(C)(C)C)cc2)c1] | 16 | >64 | 45 |
| 54 | 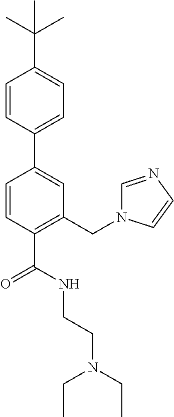 [SMILES: CCN(CC)CCNC(=O)c1ccc(-c2ccc(C(C)(C)C)cc2)cc1Cn1ccnc1] | 16 | 32 | 46 |
| 55 | 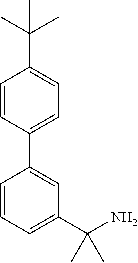 [SMILES: CC(C)(C)c1ccc(-c2cccc(C(C)(C)N)c2)cc1] | 32 | n/d | |
| 56 | 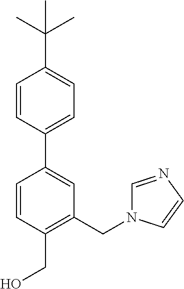 [SMILES: CC(C)(C)c1ccc(-c2ccc(CO)c(Cn3ccnc3)c2)cc1] | 16 | 16 | 47 |
| 57 | 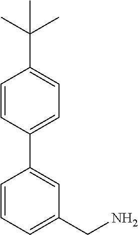 [SMILES: CC(C)(C)c1ccc(-c2cccc(CN)c2)cc1] | 16.0 | 16 | 48 |
| 58 | 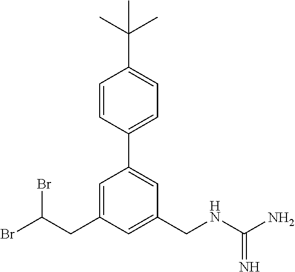 [SMILES: CC(C)(C)c1ccc(-c2cc(CNC(=N)N)cc(CC(Br)Br)c2)cc1] | 32 | n/d | 49 |
| 59 | 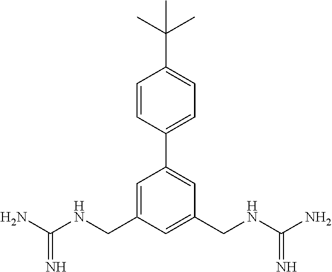 [SMILES: CC(C)(C)c1ccc(-c2cc(CNC(=N)N)cc(CNC(=N)N)c2)cc1] | 16 | 16 | 50 |
| 60 | 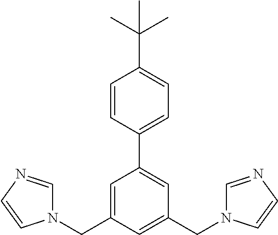 [SMILES: CC(C)(C)c1ccc(-c2cc(Cn3ccnc3)cc(Cn3ccnc3)c2)cc1] | 4.0 | 8.0 | 51 |
| 61 | 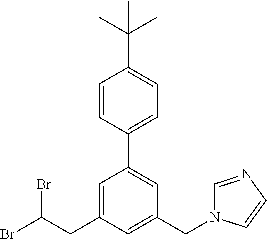 [SMILES: CC(C)(C)c1ccc(-c2cc(CC(Br)Br)cc(Cn3ccnc3)c2)cc1] | 4.0 | 8.0 | 52 |
| 62 | 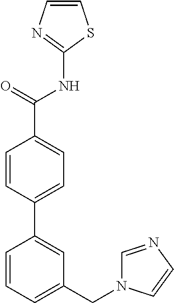 [SMILES: O=C(Nc1nccs1)c1ccc(-c2cccc(Cn3ccnc3)c2)cc1] | 4.0 | 32 | |
| 63 | 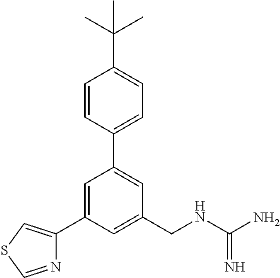 [SMILES: CC(C)(C)c1ccc(-c2cc(CNC(=N)N)cc(-c3cscn3)c2)cc1] | 2.0 | 8.0 | 53 |
| 64 | 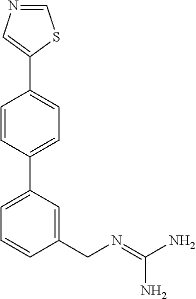 [SMILES: NC(N)=NCc1cccc(-c2ccc(-c3cncs3)cc2)c1] | 4.0 | 32 | 54 |
| 65 | 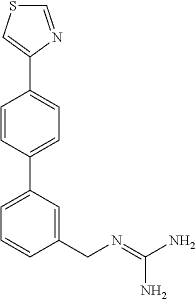 [SMILES: NC(N)=NCc1cccc(-c2ccc(-c3cscn3)cc2)c1] | 8.0 | 32 | 55 |
| 66 | 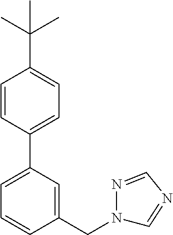 [SMILES: CC(C)(C)c1ccc(-c2cccc(Cn3cncn3)c2)cc1] | 16 | 32 | 56 |
| 67 | 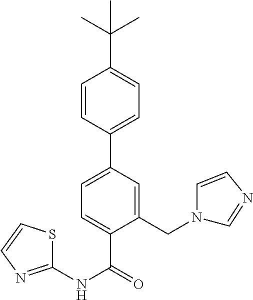 [SMILES: CC(C)(C)c1ccc(-c2ccc(C(=O)Nc3nccs3)c(Cn3ccnc3)c2)cc1] | 32 | n/d | |

*n/d = not determined

Representative compounds of the invention were also tested against vancomycin-resistant Enterococcus faecalis and Enterococcus faecium (VRE), vancomycin-sensitive Enterococcus faecalis and Enterococcus faecium (VSE). Streptococcus pyogenes, Streptococcus agalactiae, Clostridium difficile, Propionibacterium acnes, Bacillus subtilis, and Escherichia coli, and they were found to have significant antibacterial activity.

The invention will now be illustrated by the following non-limiting examples.

### EXAMPLES

### Example-1

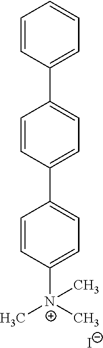 [SMILES: C[N+](C)(C)c1ccc(-c2ccc(-c3ccccc3)cc2)cc1 ; [I-]]

A solution of the N,N-dimethyl-[1,1′,4′,1″-terphenyl]-4-amine 1a (30 mg, 0.109 mmol) in iodomethane (1.0 mL) in a sealed 2-dram vial was stirred at 80° C. overnight. After being allowed to cool to room temperature, Et₂O was added to the suspension. The solid was collected by filtration to afford the desired compound (35 mg, 77%) as an off-white solid. ¹H NMR (DMSO-d₃, 300 MHz) δ 8.05 (d, J=9.0 Hz, 2H), 7.97 (d, J=9.3 Hz, 2H), 7.85 (d, J=8.7 Hz, 2H), 7.79 (d, J=8.7 Hz, 2H), 7.72 (d, J=7.2 Hz, 2H), 7.48 (t, J=7.2 Hz, 2H), 7.38 (m, 1H), 3.63 (s, 9H).

The requisite intermediate was prepared as follows:

a. Preparation of Compound

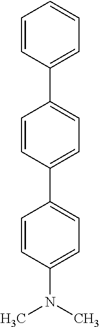 [SMILES: CN(C)c1ccc(-c2ccc(-c3ccccc3)cc2)cc1]

A 25-mL round bottom flask equipped with a magnetic stirrer, a condenser and a nitrogen in/outlet adapter was charged with 4-bromo-N,N-dimethylaniline (300 mg, 1.5 mmol), 4-biphenylboronic acid (400 mg, 2.0 mmol), water/dioxane (2 mL/6 mL), K₂CO₃ (828 mg, 6.0 mmol). The resulting solution was degassed for 5 minutes, then Pd(PPh₃)₄ (60 mg) was added. The reaction mixture was warmed to 100° C. and stirred for 3 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with EtOAc/hexanes solvent system afforded the desired compound (280 mg, 68% yield)¹H NMR (400 MHz, CDCl₃) δ 7.63 (m, 5H), 7.56 (d, J=8.0 Hz, 2H), 7.44 (t, J=8.0 Hz, 2H), 7.33 (m, 1H), 6.82 (d, J=8.0 Hz, 2H), 3.01 (s, 3H).

### Example-2

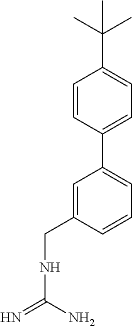 [SMILES: CC(C)(C)c1ccc(-c2cccc(CNC(=N)N)c2)cc1]

To a 10-mL vial was added di-tert-butoxycarbonyl guanidine 2d (100 mg, 0.207 mmol), CH₂Cl₂ (2.0 mL), and TFA (2.0 mL). The sealed vial was stirred at room temperature overnight. The solvent was removed and the residue was purified on silica gel. Elution using CH₂Cl₂ and a gradient to MeOH/CHCl₃/ammonium hydroxide (10/89/1) afforded the title compound as a white solid (53 mg, 91% yield). ¹H NMR (300 MHz, CDCl₃) δ 7.38-7.35 (m, 61-1), 7.15 (t, 1H), 7.06 (d, 1H), 4.1 (s, 2H), 1.28 (s, 91-1). ¹³C NMR (75 MHz, CDCl₃) δ 158.4, 150.7, 141.6, 138.6, 138.0, 129.4, 127.0, 126.3, 125.9, 45.9, 34.7, 31.6. LC/MS calculated: C₁₈H₂₃N₃, 281, found: 282 (M+H).

The requisite intermediates were prepared as follows:

a. Preparation of Compound

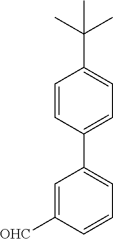 [SMILES: CC(C)(C)c1ccc(-c2cccc(C=O)c2)cc1]

A 100-mL round bottom flask equipped with a magnetic stirrer, a condenser and a nitrogen in/outlet adapter was charged with 3-bromobenzaldehyde (1.5 g, 8.10 mmol), 4-tert-butylphenylboronic acid (1.93 g, 9.70 mmol), water/dioxane (10 mL/30 ml), K₂CO₃ (2.23 g, 16.2 mmol). The resulting solution was degassed for 5 minutes, then Pd(PPh₃)₄ (180 mg, 0.162 mmol) was added. The reaction mixture was warmed to 100° C. and stirred for 3 hours. After being allowed to cool to room temperature, the reaction mixture was diluted with EtOAc (100 mL) and washed with saturated NaHCO₃ (30 mL), brine (30 mL), dried over Na₂SO₄. The organic layer was concentrated under reduced pressure and purified on silica gel. Elution with EtOAc/hexanes solvent system afforded the title compound 4′-(tert-butyl)-[1,1′-biphenyl]-3-carbaldehyde (1.6 g, 84% yield): ¹H NMR (300 MHz, CDCl₃) δ 10.12 (s, 1H), 8.13 (s, 1H), 7.88 (m, 2H), 7.63-7.52 (m, 5H), 1.40 (s, 9H). ¹³C NMR (75 MHz, CDCl₃) δ 151.4, 142.2, 137.2, 137.0, 133.2, 129.7, 128.6, 128.3, 127.0, 126.2, 34.9, 31.6. HRMS calcd for C₁₇H₁₉O (M+H)⁺, 239.1436; found, 239.1428.

b. Preparation of Compound

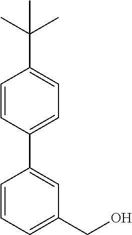 [SMILES: CC(C)(C)c1ccc(-c2cccc(CO)c2)cc1]

A 50-mL round bottom flask equipped with a magnetic stirrer was charged with the biaryl-carbaldehyde 2a (700 mg, 2.94 mmol), ethanol (95%, 10 mL), NaBH₄ (112 mg, 2.94 mmol) was added in several portions. The reaction mixture was stirred at room temperature for 1 hour. Acetone (1 mL) was added to the reaction mixture. After 20 minutes, the reaction mixture was concentrated and the residue was partitioned between EtOAc (50 mL) and 1 N HCl (15 mL) The organic layer was washed with saturated NaHCO₃ (15 mL), brine (15 mL), dried over Na₂SO₄, concentrated under reduced pressure and purified on silica gel. Elution with 10% EtOAc/Hexanes afforded the reduced compound (4′-(tert-butyl)-[1,1′-biphenyl]-3-yl)methanol (530 mg, 75% yield). ¹H NMR (400 MHz, CDCl₃) δ 7.69-7.35 (m, 8H), 4.81 (m, 2H), 1.40 (s, 9H). ¹³C NMR (100 MHz, CDCl₃) δ 150.4, 141.4, 138.0, 128.9, 126.8, 126.3, 125.7, 65.5, 34.5, 31.4.

c. Preparation of Compound

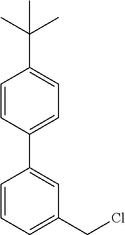 [SMILES: CC(C)(C)c1ccc(-c2cccc(CCl)c2)cc1]

A 25-mL round bottom flask equipped with a magnetic stirrer under nitrogen was charged with the benzyl alcohol 2b (600 mg, 2.50 mmol), CH₂Cl₂ (10 mL), and triethylamine (0.70 ml, 5.00 mmol). Methanesulfonyl chloride (0.39 mL, 5.00 mmol) was added via a syringe over 5 min. The resulting reaction mixture was stirred at room temperature overnight. The reaction mixture was diluted with CH₂Cl₂ (30 mL) and washed with saturated NaHCO₃ (15 ml), brine (15 mL), dried over Na₂SO₄, and concentrated under reduced pressure and purified on silica gel. Elution with hexanes afforded the product as a white solid 4′-(tert-butyl)-3-(chloromethyl)-1,1′-biphenyl (582 mg, 90% yield). ¹H NMR (400 MHz, CDCl₃) δ 7.53-7.20 (m, 8H), 4.56 (s, 2H), 1.28 (s, 9H). ¹³C NMR (100 MHz), CDCl₃) δ 150.6, 141.7, 137.8, 129.1, 127.2, 126.8, 125.8, 46.3, 34.6, 31.8.

d. Preparation of Compound

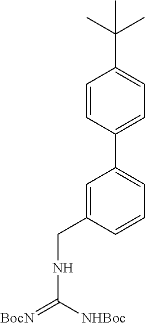

A 25-mL round bottom flask equipped with a magnetic stirrer, a condenser and a nitrogen in/outlet adapter was charged with bromomethyl intermediate 2c (130 mg, 0.50 mmol), DMF (2 mL), K₂CO₃ (103 mg, 0.75 mmol), and 1,3-bis(tert-butoxycarbonyl)guanidine (143 mg, 0.55 mmol) The reaction mixture was stirred at 50° C. for 2 hours. The reaction mixture was diluted with EtOAc (40 mL), washed with water (10 mL), 10% LiCl (10 mL), brine (10 mL), dried over Na₂SO₄, concentrated, and purified on silica gel. Elution with 5% EtOAc/hexanes afforded the title compound as a white solid 1,3-bis(t-butoxycarbonyl)guanidine (215 mg, 90% yield). ¹H NMR (400 MHz, CDCl₃) δ 7.54 (m, 3H), 7.48 (m, 3H), 7.37 (t, J=8.0 Hz, 1H), 7.22 (d, J=8.0 Hz, 1H), 5.26 (s, 2H), 1.52 (s, 9H), 1.39 (s, 9H), 1.37 (s, 9H). ¹³C NMR (100 MHz, CDCl₃) δ 150.3, 140.9, 139.3, 138.2, 128.5, 126.7, 125.8, 125.7, 125.6, 125.3, 84.0, 47.8, 34.5, 31.3, 28.3, 27.8.

### Example-3

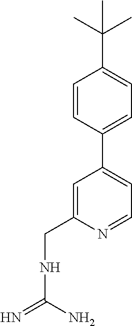 [SMILES: CC(C)(C)c1ccc(-c2ccnc(CNC(=N)N)c2)cc1]

To a 10-mL vial was added di-tert-butoxycarbonyl guanidine 3d (35 mg, 0.072 mmol), CH₂Cl₂ (0.5 mL), and TFA (0.5 mL). The sealed vial was stirred at 50° C. for 2 hours. The solvent was removed and the residue was purified on silica gel. Elution with CH₂Cl₂ to (10/89/1: MeOH/CHCl₃/ammonium hydroxide) afforded the title compound as a white solid (18 mg, 89% yield).: ¹H NMR (300 MHz, (CDCl₃) δ 8.32 (s, 1H), 7.45-7.20 (m, 7H), 6.43 (bs, 2H), 4.32 (s, 2H), 1.27 (s, 9H). ¹³C NMR (75 MHz, CDCl₃) δ 159.1, 157.2, 152.8, 149.7, 149.3, 134.5, 126.9, 126.3, 120.6, 120.4, 47.5, 34.9, 31.4. LC/MS calculated: C₁₇H₂₂N₄, 282, found: 283 (M+H).

The requisite intermediates were prepared as follows:

a. Preparation of Compound

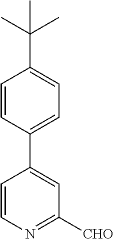 [SMILES: CC(C)(C)c1ccc(-c2ccnc(C=O)c2)cc1]

A 50-mL round bottom flask equipped with a magnetic stirrer, a condenser and a nitrogen in/outlet adapter was charged with 4-chloropicolinaldehyde (1.15 g, 8.10 mmol), 4-tert-butylphenylboronic acid (1.93 g, 9.70 mmol), water/dioxane (10 mL/30 ml), K₂CO₃ (2.23 g, 16.2 mmol). The resulting solution was degassed for 5 minutes, then Pd(PPh₃)₄ (180 mg, 0.162 mmol) was added. The reaction mixture was warmed to 100° C. and stirred for 3 hours. After being allowed to cool to room temperature, the reaction mixture was diluted with EtOAc (100 mL) and washed with saturated NaHCO₃ (30 mL), brine (30 mL), dried over Na₂SO₄. The organic layer was concentrated under reduced pressure and purified on silica gel. Elution with EtOAc/hexanes solvent system afforded the title compound (1.59 g, 82% yield). ¹H NMR (300 MHz, CDCl₃) δ 10.15 (s, 1H), 8.81 (d, J=6.0 Hz, 1H), 8.21 (d, J=3.0 Hz, 1H), 7.74 (dd, J=6.0, 3.0 Hz, 1H), 7.65 (m, 2H), 7.54 (m, 2H), 1.37 (s, 9H). ¹³C NMR (75 MHz, CDCl₃) δ 193.8, 153.8, 153.4, 150.8, 149.7, 134.2, 126.9, 126.5, 125.5, 119.5, 35.0, 31.4.

b. Preparation of Compound

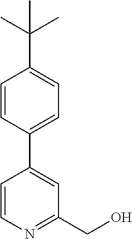 [SMILES: CC(C)(C)c1ccc(-c2ccnc(CO)c2)cc1]

A 50-mL round bottom flask equipped with a magnetic stirrer was charged biaryl-carbaldehyde 3a (2.94 mmol), ethanol (95%, 10 mL), NaBH₄ (112 mg, 2.94 mmol) was added in several portions. The reaction mixture was stirred at room temperature for 1 hour. Acetone (1 mL) was added to the reaction mixture. After 20 minutes, the reaction mixture was concentrated and the residue was partitioned between EtOAc (50 mL) and 1 N HCl (15 mL). The organic layer was washed with saturated NaHCO₃ (15 mL), brine (15 mL), dried over Na₂SO₄, concentrated under reduced pressure and purified on silica gel. Elution with 10% EtOAc/Hexanes afforded the reduced compounds in good yield: ¹H NMR (300 MHz, CDCl₃) δ 8.57 (d, J=3.0 Hz, 1H), 7.60 (d, J=6.0 Hz, 2H), 7.58-7.41 (m, 4H), 4.83 (s, 2H), 1.37 (s, 9H). ¹³C NMR (75 MHz, CDCl₃) 149.1, 135.3, 126.9, 126.3, 120.6, 118.4, 64.6, 34.9, 31.5. LC/MS calculated: C₁₆H₁₈NO, 241, found: 242 (M+H).

c. Preparation of Compound

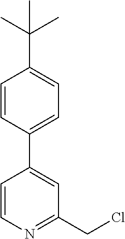 [SMILES: CC(C)(C)c1ccc(-c2ccnc(CCl)c2)cc1]

A 25-mL round bottom flask equipped with a magnetic stirrer under nitrogen was charged with the benzyl alcohol 3b (2.50 mmol), CH₂Cl₂ (10 mL), and triethylamine (0.70 ml, 5.00 mmol). Methanesulfonyl chloride (0.39 mL, 5.00 mmol) was added via a syringe over 5 min. The resulting reaction mixture was stirred at room temperature overnight. The reaction mixture was diluted with CH₂Cl₂ (30 mL) and washed with saturated NaHCO₃ (15 ml), brine (15 mL), dried over Na₂SO₄, and concentrated under reduced pressure and purified on silica gel. Elution with hexanes afforded the product as a white solid. ¹H NMR (300 MHz, CDCl₃) δ 8.60 (J=6.0 Hz, 1H), 7.58-7.25 (m, 6H), 4.73 (s, 2H), 1.37 (s, 9H). ¹³C NMR (75 MHz, CDCl₃) δ 157.3, 152.9, 150.1, 149.7, 135.1, 127.0, 126.4, 121.1, 120.8, 47.1, 35.0, 31.5.

d. Preparation of Compound

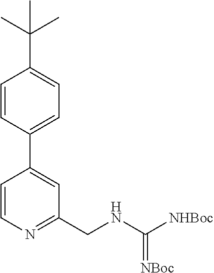

A 25-mL round bottom flask equipped with a magnetic stirrer, a condenser and a nitrogen in/outlet adapter was charged with chloromethyl intermediate 3c (130 mg, 0.50 mmol), DMF (2 mL), K₂CO₃ (103 mg, 0.75 mmol), and 1,3-bis(tert-butoxycarbonyl)guanidine (143 mg, 0.55 mmol) The reaction mixture was stirred at 50° C. for 2 hours. The reaction mixture was diluted with EtOAc (40 mL), washed with water (10 mL), 10% LiCl (10 mL), brine (10 mL), dried over Na₂SO₄, concentrated, and purified on silica gel. Elution with 5% EtOAc/hexanes afforded the title compound as a white solid. ¹H NMR (300 MHz, CDCl₃) δ 9.5 (bs, 2H), 8.47 (d, J=6.0 Hz, 1H), 7.50-7.42 (m, 4H), 7.3 (m, 2H), 5.4 (s, 2H), 1.47 (s, 9H), 1.36 (s, 9H), 1.25 (s, 9H). ¹³C NMR (75 MHz, CDCl₃) δ 163.8, 161.2, 159.3, 155.2, 152.6, 149.6, 148.8, 135.6, 126.9, 126.3, 119.9, 118.0, 84.2, 79.2, 49.7, 34.9, 31.5, 28.5, 28.3, 27.9.

### Example-4

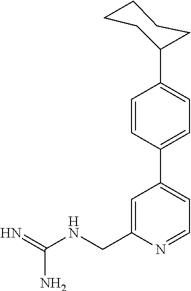 [SMILES: N=C(N)NCc1cc(-c2ccc(C3CCCCC3)cc2)ccn1]

To a 10-mL vial was added di-tert-butoxycarbonyl guanidine 4c (60 mg, 0.12 mmol), CH₂Cl₂ (1.5 mL), and TFA (1.5 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound (33 mg, 91% yield). LC/MS 309 [M+H].

The requisite intermediates were prepared as follows:

a. Preparation of Compound

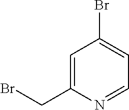

To a solution of 4-bromo-2-methylpyridine (1.0 g) in carbon tetrachloride (10.0 mL) was added NBS (1.25 g) and AIBN (50 mg) and the mixture was stirred at 85° C. for 1 hour. After cooling, the reaction mixture was filtered to remove the insoluble materials and the filtrate was concentrated. The crude material (120 mg) was used for the next step immediately. ¹H NMR (400 MHz, CDCl₃) δ 8.32 (d, J=4.0 Hz, 1H), 7.57 (s, 1H), 7.33 (d, J=4.0 Hz, 1H), 4.43 (s, 2H).

b. Preparation of Compound

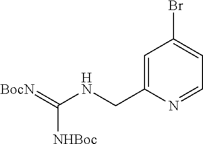

A 25-mL round bottom flask equipped with a magnetic stirrer, was charged with the bromo intermediate 4a (150 mg, 0.6 mmol), DMF (3 mL), K₂CO₃ (180 mg, 1.3 mmol), and 1,3-bis(tert-butoxycarbonyl)guanidine (330 mg, 1.21 mmol). The reaction mixture was stirred at 50° C. for 2 hours. The reaction mixture was diluted with EtOAc, washed with water, 10% LiCl, brine, dried over Na₂SO₄, concentrated, and purified on silica gel. Elution with 5% EtOAc/hexanes afforded the desired compound (120 mg, 47% yield). ¹H NMR (300 MHz, CDCl₃) δ 8.27 (s, 1H), 7.24 (m, 2H), 5.25 (s, 2H), 1.40 (s, 9H), 1.21 (s, 9H).

c. Preparation of Compound

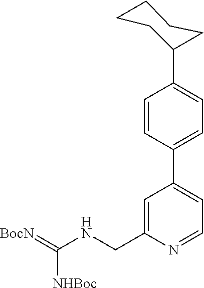

A 25-mL round bottom flask equipped with a magnetic stirrer, a condenser and a nitrogen in/outlet adapter was charged with the bromo pyridyl compound 4b (120 mg, 0.28 mmol), (4-cyclohexylphenyl)boronic acid (114 mg, 0.558 mmol), water/dioxane (2 mL/6 mL), K₂CO₃ (138 mg, 1.0 mmol). The resulting solution was degassed for 5 minutes, then Pd(PPh₃)₄ (60 mg) was added. The reaction mixture was warmed to 100° C. and stirred for 3 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with EtOAc/hexanes afforded the desired compound (77 mg, 54% yield). ¹H NMR (400 MHz, CDCl₃) δ 9.51 (bs, 2H), 8.55 (d, J=4.0 Hz, 1H), 8.12 (bs, 1H), 7.56 (d, J=8.0 Hz, 2H), 7.33 (m, 4H), 5.41 (s, 2H), 2.59 (m, 1H), 1.94-1.81 (m, 9H), 1.49 (s, 9H), 1.28 (s, 9H).

### Example-5

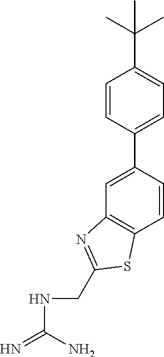 [SMILES: CC(C)(C)c1ccc(-c2ccc3sc(CNC(=N)N)nc3c2)cc1]

A 10-mL vial was added di-tert-butoxycarbonyl guanidine 5c (10 mg, 0.0185 mmol), CH₂Cl₂ (1 mL), and TFA (0.25 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the desired compound (4.0 mg, 64% yield), LC/MS: 339.5 (M+H).

The requisite intermediates were prepared as follows:

a. Preparation of Compound

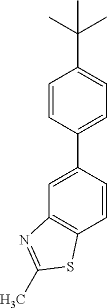 [SMILES: Cc1nc2cc(-c3ccc(C(C)(C)C)cc3)ccc2s1]

To a solution of 5-chloro-2-methylbenzthiazole (400 mg) in DMF-H₂O (12.0 mL-6.0 mL) was added t-butylphenylboronic acid (462 mg) and potassium phosphate (560 mg). The reaction mixture was degassed for 20 minutes followed by addition of Pd(dppf)Cl₂.DCM (260 mg). The reaction was again degassed for 20 minutes and then heated at 110° C. for 2 hours under argon. After being allowed to cool to room temperature, the reaction mixture was diluted with EtOAc and washed with saturated NaHCO₃, then brine and dried over Na₂SO₄. The organic layer was concentrated under reduced pressure and purified on silica gel. Elution with EtOAc/hexanes solvent system afforded the desired compound (220 mg) as an inseparable mixture together with the starting material.

b. Preparation of Compound

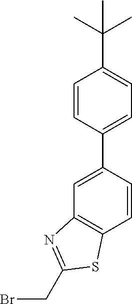 [SMILES: CC(C)(C)c1ccc(-c2ccc3sc(CBr)nc3c2)cc1]

A mixture of above mixture (200 mg), NBS (212 mg, 1.18 mmol) in carbon tetrachloride (4.0 mL) was heated under light for 2 hours. The solids were filtered and the solvent was removed to give the crude product. Purification using 10% ethyl acetate in hexane afforded the product along with dibromo and starting material as a mixture.

c. Preparation of Compound

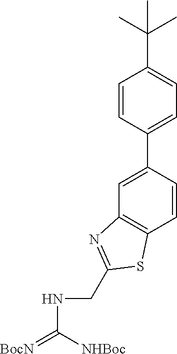

A 25-mL round bottom flask equipped with a magnetic stirrer, a condenser was charged with bromomethyl intermediate as a mixture together with the dibromo derivative (37 mg, 0.102 mmol), DMF (3 mL), K₂CO₃ (30 mg, 0.202 mmol), and 1,3-bis(tert-butoxycarbonyl)guanidine (34 mg, 0.115 mmol) The reaction mixture was stirred at 50° C. for 12 hours. The reaction mixture was diluted with EtOAc, washed with water, 10% LiCl, brine, dried over Na₂SO₄, concentrated and purified on silica gel. Elution with 10% EtOAc in hexane afforded the desired compound (11 mg). ¹H NMR (300 MHz, CDCl₃) δ 8.18 (s, 1H), 7.88 (d, J=7.2 Hz, 1H), 7.63-7.61 (m, 3H), 7.50 (d, J=6.9 Hz, 2H), 5.62 (s, 2H), 1.48 (s, 9H), 1.37 (s, 9H), 1.35 (s, 9H).

### Example-6

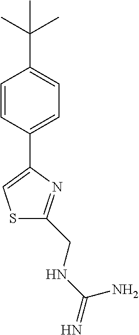 [SMILES: CC(C)(C)c1ccc(-c2csc(CNC(=N)N)n2)cc1]

To a 10-mL vial was added di-tert-butoxycarbonyl guanidine 6c (20 mg, 0.041 mmol), CH₂Cl₂ (1.0 mL), and TFA (0.5 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the desired compound (10 mg, 85% yield). ¹H NMR (300 MHz, CDCl₃) δ 7.62 (d, J=8.1 Hz, 2H), 7.38 (d, J=8.1 Hz, 2H), 7.31 (s, 1H), 4.50 (d, J=5.7 Hz, 2H), 1.3 (s, 9H).

The requisite intermediates were prepared as follows:

a. Preparation of Compound

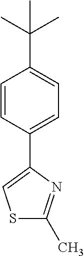 [SMILES: Cc1nc(-c2ccc(C(C)(C)C)cc2)cs1]

A 25-mL round bottom flask equipped with a magnetic stirrer, a condenser and a nitrogen in/outlet adapter was charged with 4-bromo-2-methylthiazole (250 mg, 1.4 mmol), 4-tert-butylphenylboronic acid (400 mg, 2.1 mmol), water/dioxane (1 mL/3 ml), K₂CO₃ (386 mg, 2.8 mmol). The resulting solution was degassed for 5 minutes, then Pd(PPh₃)₄ (30 mg) was added. The reaction mixture was warmed to 100° C. and stirred for 3 hours. After being allowed to cool to room temperature, the reaction mixture was diluted with EtOAc and washed with saturated NaHCO₃, then brine and dried over Na₂SO₄. The organic layer was concentrated under reduced pressure and purified on silica gel. Elution with EtOAc/hexanes solvent system afforded the desired compound (210 mg, 65% yield). ¹H NMR (300 MHz, CDCl₃) δ 7.79 (d, J=7.8 Hz, 2H), 7.42 (d, J=7.8 hz, 2H), 7.26 (s, 1H), 2.77 (s, 3H), 1.34 (s, 3H).

b. Preparation of Compound

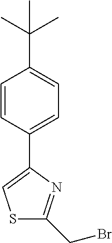 [SMILES: CC(C)(C)c1ccc(-c2csc(CBr)n2)cc1]

A mixture of above compound 6a (210 mg, 0.91 mmol), NBS (212 mg, 1.18 mmol) in carbon tetrachloride (4.0 mL) was heated under light for 2 hours. The solids were filtered and the solvent was removed to give the crude product. Purification using 10% ethyl acetate in hexane afforded the product along with dibromo and starting material as a mixture.

c. Preparation of Compound

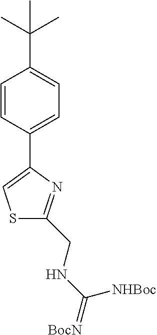

A 25-mL round bottom flask equipped with a magnetic stirrer, a condenser was charged with above mixture (70 mg), DMF (3 mL), K₂CO₃ (62 mg, 0.451 mmol), and 1,3-bis(tert-butoxycarbonyl)guanidine (75 mg, 0.292 mmol) The reaction mixture was stirred at 50° C. for 12 hours. The reaction mixture was diluted with EtOAc, washed with water, 10% LiCl, brine, dried over Na₂SO₄, concentrated, and purified on silica gel. Elution with 100% EtOAc afforded the desired compound (50 mg, 45% yield). ¹H NMR (300 MHz, CDCl₃) δ 7.76 (d, J=8.1 Hz, 2H), 7.45 (d, J=7.8 Hz, 2H), 7.40 (s, 1H), 4.68 (s, 2H), 1.48 (s, 9H), 1.38 (s, 9H), 1.27 (s, 9H).

### Example-7

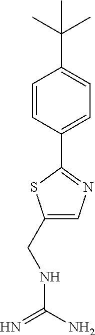 [SMILES: CC(C)(C)c1ccc(-c2ncc(CNC(=N)N)s2)cc1]

To a 10-mL vial was added di-tert-butoxycarbonyl guanidine 7c (68 mg, 0.135 mmol), CH₂Cl₂ (1 mL), and TFA (1 mL) The sealed vial was stirred at room temperature overnight. The solvent was removed and the residue was purified on silica gel. Elution with CH₂Cl₂ to (10/89/1: MeOH/CHCl₃/ammonium hydroxide) afforded the desired compound (37 mg, 92% yield). ¹H NMR (300 MHz, DMSO-d₆) δ 8.3 (s, 1H), 7.57 (d, J=8.4 Hz, 2H), 7.28 (d, J=8.7 Hz, 2H), 4.4 (s, 2H), 1.21 (s, 9H).

The requisite intermediates were prepared as follows:

a. Preparation of Compound

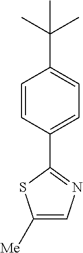 [SMILES: Cc1cnc(-c2ccc(C(C)(C)C)cc2)s1]

A 25-mL round bottom flask equipped with a magnetic stirrer, a condenser and a nitrogen in/outlet adapter was charged with 2-bromo-5-methylthiazole (250 mg, 1.4 mmol), 4-tert-butylphenylboronic acid (400 mg, 2.1 mmol), water/dioxane (1 mL/3 ml), K₂CO₃ (386 mg, 0.2 mmol). The resulting solution was degassed for 5 minutes, then Pd(PPh₃)₄ (30 mg) was added. The reaction mixture was warmed to 100° C. and stirred for 3 hours. After being allowed to cool to room temperature, the reaction mixture was diluted with EtOAc (100 mL) and washed with saturated NaHCO₃, brine and then dried over Na₂SO₄. The organic layer was concentrated under reduced pressure and purified on silica gel. Elution with EtOAc/hexanes solvent system afforded the desired compound (145 mg, 45% yield). ¹H NMR (300 MHz, CDCl₃) δ 7.81 (d, J=9.0 Hz, 2H), 7.47 (s, 1H), 7.43 (d, J=9.0 Hz, 2H), 2.50 (s, 3H), 1.34 (s, 9H).

b. Preparation of Compound

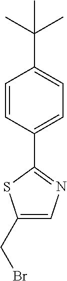 [SMILES: CC(C)(C)c1ccc(-c2ncc(CBr)s2)cc1]

A mixture of the substituted methylthiazole 7a (145 mg, 0.63 mmol), NBS (145 mg, 0.819 mmol) in carbon tetrachloride (5.0 mL) was heated under light for 2 hours. The solids were filtered and the solvent was removed to give the crude product. Purification using 10% ethyl acetate in hexane afforded the product (55 mg, 28% yield). ¹H NMR (300 MHz, CDCl₃) δ 7.85 (d, J=8.1 Hz, 2H), 7.76 (s, 1H), 7.46 (d, J=8.1 Hz, 2H), 4.76 (s, 2H), 1.35 (s, 9H).

c. Preparation of Compound

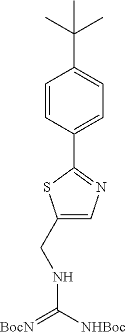

A 25-mL round bottom flask equipped with a magnetic stirrer was charged with bromomethyl intermediate 7b (50 mg, 0.161 mmol), DMF (2 mL), K₂CO₃ (51 mg, 0.37 mmol), and 1,3-bis(tert-butoxycarbonyl)guanidine (70 mg, 0.27 mmol) The reaction mixture was stirred at 50° C. for 2 hours. The reaction mixture was diluted with EtOAc, washed with water, 10% LiCl, brine, dried over Na₂SO₄, concentrated, and purified on silica gel. Elution with 5% EtOAc/hexanes afforded the title compound as a fluffy white solid (68 mg, 87% yield). ¹H NMR (300 MHz, CDCl₃) δ 7.84 (d, J=8.4 Hz, 2H), 7.81 (s, 1H), 7.45 (d, J=8.7 Hz, 21-1), 5.26 (s, 2H), 1.56-1.34 (3×9H).

### Example-8

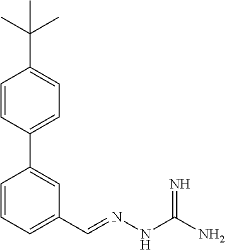 [SMILES: CC(C)(C)c1ccc(-c2cccc(/C=N/NC(=N)N)c2)cc1]

A mixture of biaryl aldehyde 2a (100 mg, 0.42 mmol), aminoguanidine hydrochloride (90 mg, 0.84 mmol) in 5 mL EtOH was refluxed overnight. The resulting solid was filtered, washed with ether and was dried to produce the desired compound in high purity (80 mg, 65% yield). ¹H NMR (300 MHz, DMSO-d6) δ 8.26 (s, 1H), 8.19 (s, 1H), 7.82 (d, J=7.5 Hz, 1H), 7.75 (d, J=8.1 Hz, 1H), 7.78 (d, J=8.7 Hz, 2H), 7.54 (m, 3H), 1.34 (s, 9H).

### Example-9

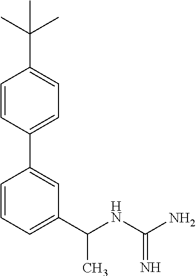 [SMILES: CC(NC(=N)N)c1cccc(-c2ccc(C(C)(C)C)cc2)c1]

A 10-mL vial was added di-tert-butoxycarbonyl guanidine 9c (100 mg, 0.20 mmol), CH₂Cl₂ (2 mL), and TFA (1 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the desired compound (52 mg, 87% yield). ¹H NMR (300 MHz, CD₃OD) δ 7.59 (d, J=8.1 Hz, 4H), 7.53-7.46 (m, 3H), 7.33 (d, J=8.1 Hz, 1H), 4.80 (m, 1H), 1.61 (d, J=6.9 Hz, 3H), 1.39 (s, 9H).

The required intermediates were prepared as follows:

a. Preparation of Compound

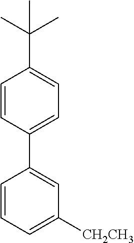 [SMILES: CCc1cccc(-c2ccc(C(C)(C)C)cc2)c1]

A 25-mL round bottom flask equipped with a magnetic stirrer, a condenser and a nitrogen in/outlet adapter was charged with 1-bromo-3-ethylbenzene (250 mg, 1.35 mmol), 4-tert-butylphenylboronic acid (400 mg, 2.1 mmol), water/dioxane (1 mL/3 ml), K₂CO₃ (386 mg, 2.0 mmol). The resulting solution was degassed for 5 minutes, then Pd(PPh₃)₄ (30 mg) was added. The reaction mixture was warmed to 100° C. and stirred for 3 h. After being allowed to cool to room temperature, the reaction mixture was diluted with EtOAc (100 mL) and washed with saturated NaHCO₃, brine, dried over Na₂SO₄. The organic layer was concentrated under reduced pressure and purified on silica gel. Elution with EtOAc/hexanes solvent system afforded the desired compound (260 mg, 81% yield). ¹H NMR (300 MHz, CDCl₃) δ 7.55-7.31 (m, 7H), 7.16 (d, J=7.6 Hz, 1H), 2.70 (qt, 2H), 1.36 (s, 9H), 1.27 (t, J=7.5 Hz, 3H).

b. Preparation of Compound

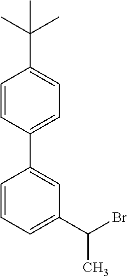 [SMILES: CC(Br)c1cccc(-c2ccc(C(C)(C)C)cc2)c1]

A mixture of the substituted ethyl biaryl compound 9a (145 mg, 0.63 mmol), NBS (145 mg, 0.819 mmol) in carbon tetrachloride (5.0 mL) was heated under light for 2 hours. The solids were filtered and the solvent was removed to give the crude product. Purification using 10% ethyl acetate in hexane afforded the product (55 mg, 28% yield). ¹H NMR (300 MHz, CDCl₃) δ 7.64-7.41 (m, 8H), 5.29 (m, 1H), 2.09 (d, J=7.2 Hz, 3H), 1.37 (s, 9H).

c. Preparation of Compound

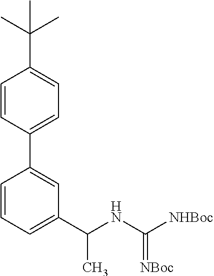

A 25-mL round bottom flask equipped with a magnetic stirrer, a condenser and a nitrogen in/outlet adapter was charged with bromomethyl intermediate 9b (257 mg, 1.233 mmol), DMF (2 mL), K₂CO₃ (280 mg, 2.0 mmol), and 1,3-bis(tert-butoxycarbonyl)guanidine (402 mg, 1.55 mmol) The reaction mixture was stirred at 50° C. for 12 hours. The reaction mixture was diluted with EtOAc, washed with water, 10% LiCl, brine and then dried over Na₂SO₄, concentrated, and purified on silica gel. Elution with 5% EtOAc/hexanes afforded the title compound (180 mg, 30% yield). ¹H NMR (400 MHz, CDCl₃) δ 7.51-7.15 (m, 8H), 6.66 (m, 1H), 1.75-1.11 (30H).

### Example-10

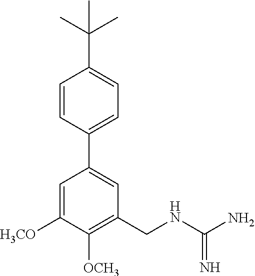 [SMILES: COc1cc(-c2ccc(C(C)(C)C)cc2)cc(CNC(=N)N)c1OC]

A 10-mL vial was added di-tert-butoxycarbonyl guanidine 10d (60 mg, 0.129 mmol), CH₂Cl₂ (1 mL), and TFA (1 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound (33 mg, 75% yield). ¹H NMR (300 MHz, CDCl₃) δ 7.47 (s, 4H), 7.08 (m, 2H), 4.24 (s, 2H), 3.92 (s, 3H), 3.90 (s, 3H), 1.37 (s, 9H).

The requisite intermediates were prepared as follows:

a. Preparation of Compound

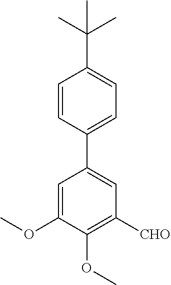 [SMILES: COc1cc(-c2ccc(C(C)(C)C)cc2)cc(C=O)c1OC]

A 25-mL round bottom flask equipped with a magnetic stirrer, a condenser and a nitrogen in/outlet adapter was charged with 5-bromo-2,3-dimethoxybenzaldehyde (500 mg, 2.0 mmol), 4-tert-butylphenylboronic acid (428 mg, 2.1 mmol), water/dioxane (2 mL/6 ml), K₂CO₃ (441 mg, 3.2 mmol). The resulting solution was degassed for 5 minutes, then Pd(PPh₃)₄ (60 mg) was added. The reaction mixture was warmed to 100° C. and stirred for 3 hours. After being allowed to cool to room temperature, the reaction mixture was diluted with EtOAc (100 mL), washed with saturated NaHCO₃, brine and dried over Na₂SO₄. The organic layer was concentrated under reduced pressure and purified on silica gel. Elution with EtOAc/hexanes solvent system afforded the desired compound (340 mg, 57% yield). ¹H NMR (300 MHz, CDCl₃) δ 10.35 (s, 1H), 7.52 (s, 1H), 7.42-7.33 (m, 4H), 7.24 (s, 1H), 3.90 (s, 3H), 3.84 (s, 3H), 1.36 (s, 9H).

b. Preparation of Compound

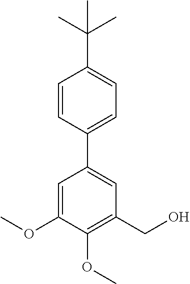 [SMILES: COc1cc(-c2ccc(C(C)(C)C)cc2)cc(CO)c1OC]

A 50-mL round bottom flask equipped with a magnetic stirrer was charged with the biaryl-carbaldehyde 10a (320 mg, 1.072 mmol), ethanol (95%, 10 mL), NaBH₄ (112 mg, 2.94 mmol) was added in several portions. The reaction mixture was stirred at room temperature for 1 hour. Acetone (1 mL) was added to the reaction mixture. After 20 minutes, the reaction mixture was concentrated and the residue was partition between EtOAc (50 mL) and 1 N HCl (15 mL). The organic layer was washed with saturated NaHCO₃ (15 mL), brine (15 mL), dried over Na₂SO₄, concentrated under reduced and purified on silica gel. Elution with 50% EtOAc/Hexanes afforded the reduced compound (220 mg, 68% yield). ¹H NMR (300 MHz, CDCl₃) δ 7.44 (m, 4H), 7.12 (s, 1H), 7.05 (s, 1H), 4.72 (s, 2H), 3.91 (s, 3H), 3.88 (s, 3H), 1.34 (s, 9H).

c. Preparation of Compound

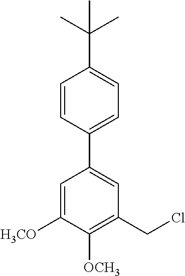 [SMILES: COc1cc(-c2ccc(C(C)(C)C)cc2)cc(CCl)c1OC]

A 25-mL round bottom flask equipped with a magnetic stirrer was charged with the alcohol 10b (100 mg, 0.41 mmol), CH₂Cl₂ (3.0 mL), and triethylamine (0.110 mL, 0.8 mmol) under nitrogen. Methanesulfonyl chloride (0.050 mL, 0.062 mmol) was added via a syringe over 5 minutes. The resulting reaction mixture was stirred at room temperature overnight. The reaction mixture was diluted with CH₂Cl₂ and washed with saturated NaHCO₃, brine, and then dried over Na₂SO₄, and concentrated under reduced pressure and purified on silica gel. Elution with hexanes afforded the product (85 mg, 65% yield). ¹H NMR (300 MHz, CDCl₃) δ 7.55-7.48 (m, 4H), 7.22 (s, 1H), 7.13 (s, 1H), 4.74 (s, 2H), 4.00 (s, 3H), 3.97 (s, 3H), 1.40 (s, 9H).

d. Preparation of Compound

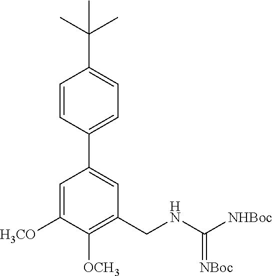

A 25-mL round bottom flask equipped with a magnetic stirrer was charged with chloro intermediate 10c (55 mg, 0.145 mmol), DMF (2 mL), K₂CO₃ (40 mg, 0.3 mmol), and 1,3-bis(tert-butoxycarbonyl)guanidine (56 mg, 0.21 mmol) The reaction mixture was stirred at 50° C. for 2 hours. The reaction mixture was diluted with EtOAc, washed with water, 10% LiCl, brine, dried over Na₂SO₄, concentrated, and purified on silica gel. Elution with 5% EtOAc/hexanes afforded the desired compound (60 mg, 77% yield); ¹H NMR (300 MHz, CDCl₃) δ 7.47 (s, 4H), 7.02 (d, J=1.8 Hz, 1H), 6.88 (d, J=2.1 Hz, 1H), 5.34 (s, 2H), 3.94 (s, 3H), 3.89 (s, 3H), 1.48 (s, 9H), 1.39 (s, 9H), 1.34 (s, 9H).

### Example-11

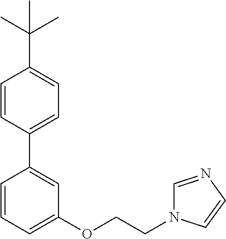 [SMILES: CC(C)(C)c1ccc(-c2cccc(OCCn3ccnc3)c2)cc1]

To a solution of 2-((4′-(tert-butyl)-(1:1′-biphenyl]-3-yl)oxy)ethyl methanesulfonate 13c (30 mg, 0.086 mmol) in 2.0 ml ACN was added excess imidazole (50 mg) and the reaction mixture was stirred at room temperature for 12 hours. The solvent was removed and the residue was purified eluting with CH₂Cl₂ to (10/89/1: MeOH/CHCl₃/ammonium hydroxide) afforded the desired compound (12 mg, 43% yield). ¹H NMR (400 MHz, CDCl₃) δ 7.64 (s, 1H), 7.5 (m, 4H), 7.35 (m, 1H), 7.22 (s, 1H), 7.1 (m, 3H), 6.85 (d, 1H), 4.38 (t, 2H), 4.3 (t, 2H), 1.48 (s, 9H).

### Example-12

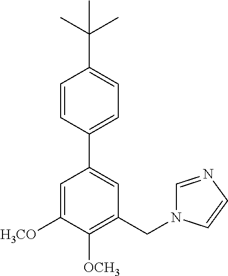 [SMILES: COc1cc(-c2ccc(C(C)(C)C)cc2)cc(Cn2ccnc2)c1OC]

To a solution of chloro intermediate 10c (30 mg 0.094 mmol) in 1.0 mL DMF was added excess imidazole. The solution was stirred at room temperature for 12 hours. The reaction mixture was diluted with EtOAc, washed with water, 10% LiCl, brine, dried over Na₂SO₄, concentrated, and purified on silica gel. Elution with 5% EtOAc/hexanes afforded the desired compound (17 mg, 51% yield), ¹H NMR (300 MHz, CD3OD) δ 7.78 (s, 1H), 7.53-7.45 (m, 4H), 7.24 (d, J=2.1 Hz, 1H), 7.15 (s, 1H), 7.05 (d, J=2.1 Hz, 1H), 6.98 (s, 1H), 5.26 (s, 2H), 3.92 (s, 3H), 3.79 (s, 3H), 1.36 (s, 9H).

### Example-13

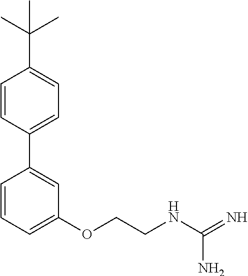 [SMILES: CC(C)(C)c1ccc(-c2cccc(OCCNC(=N)N)c2)cc1]

To a 10-mL vial was added di-tert-butoxycarbonyl guanidine 13d (40 mg, 0.083 mmol), CH₂Cl₂ (0.5 mL), and TFA (0.5 mL). The sealed vial was stirred at 50° C. for 2 hours. The solvent was removed and the residue was purified on silica gel. Elution with CH₂Cl₂ to (10/89/1: MeOH/CHCl₃/ammonium hydroxide) afforded the desired compound (20 mg, 77% yield). ¹H NMR (300 MHz, CD3OD) δ 7.55 (d, J=8.4 Hz, 2H), 7.49 (d, J=8.4 Hz, 2H), 7.37 (t, J=7.8 Hz, 1H), 7.23 (m, 2H), 6.94 (m, 1H), 4.21 (t, J=5.4 Hz, 2H), 3.65 (t, J=4.8 Hz, 2H), 1.37 (s, 9H).

The requisite intermediates were prepared as follows:

a. Preparation of Compound

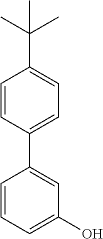 [SMILES: CC(C)(C)c1ccc(-c2cccc(O)c2)cc1]

A 25-mL round bottom flask equipped with a magnetic stirrer, a condenser and a nitrogen in/outlet adapter was charged with 3-bromophenol (519 mg, 3.0 mmol), 4-tert-butylphenylboronic acid (800 mg, 4.2 mmol), water/dioxane (2 mL/6 mL), K₂CO₃ (828 mg, 6.0 mmol). The resulting solution was degassed for 5 min, then Pd(PPh₃)₄ (60 mg) was added. The reaction mixture was warmed to 100° C. and stirred for 3 hours. After being allowed to cool to room temperature, the reaction mixture was diluted with EtOAc, washed with saturated NaHCO₃, then brine and dried over Na₂SO₄. The organic layer was concentrated under reduced pressure and purified on silica gel. Elution with EtOAc/hexanes solvent system afforded the desired compound (580 mg, 85% yield). 7.48 (d, J=6.0 Hz, 2H), 7.43 (d, J=6.0 Hz, 2H), 7.23 (m, 1H), 7.14 (m, 1H), 7.02 (s, 1H), 6.77 (m, 1H), 1.40 (s, 9H).

b. Preparation of Compound

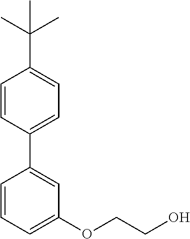 [SMILES: CC(C)(C)c1ccc(-c2cccc(OCCO)c2)cc1]

To a solution of 3-hydroxy-4′-tert-butyl[1:1′]biphenyl 13a (580 mg, 2.5 mmol) in 3.0 mL DMF was added 2-bromoethanol (0.36 mL, 5.0 mmol) and K₂CO₃ (690 mg, 5.0 mmol) and the resulting mixture was stirred at room temperature for 12 hours. The reaction mixture was diluted with ethyl acetate and was washed with water and brine. Removal of the solvent and purification using silica gel produced the desired compound (500 mg, 74% yield). ¹H NMR (300 MHz, CDCl₃) δ 7.56 (d, J=9.0 Hz, 2H), 7.58 (d, J=9.0 Hz, 2H), 7.38 (t, J=6.0 Hz, 1H), 7.28 (m, 1H), 6.92 (m, 2H), 6.92 (d, J=9.0 Hz, 1H), 4.18 (m, 2H), 4.03 (m, 2H), 1.40 (s, 9H).

c. Preparation of Compound

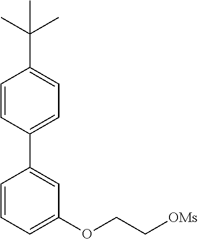 [SMILES: CC(C)(C)c1ccc(-c2cccc(OCCOS(C)(=O)=O)c2)cc1]

A 25-mL round bottom flask equipped with a magnetic stirrer was charged with the alcohol 13b (70 mg, 0.259 mmol), CH₂Cl₂ (3 mL), and triethylamine (0.10 ml, 0.71 mmol) under nitrogen. Methanesulfonyl chloride (0.055 mL, 0.71 mmol) was added via a syringe over 5 minutes. The resulting reaction mixture was stirred at room temperature overnight. The reaction mixture was diluted with CH₂Cl₂ and washed with saturated NaHCO₃, brine, dried over Na₂SO₄, and concentrated under reduced pressure and purified on silica gel. Elution with hexanes afforded the product (70 mg, 78% yield). ¹H NMR (300 MHz, CDCl₃) δ 7.56 (d, J=9.0 Hz, 2H), 7.49 (d, J=9.0 Hz, 2H), 7.39 (t, J=6.0 Hz, 1H), 7.265 (m, 1H), 7.15 (s, 1H), 6.92-6.88 (m, 1H), 4.64 (m, 2H), 4.34 (m, 2H), 3.14 (s, 3H), 1.40 (s, 3H).

c. Preparation of Compound

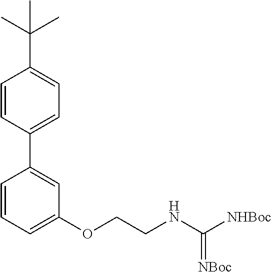

A 25-mL round bottom flask equipped with a magnetic stirrer, was charged with mesylate 13c (58 mg, 0.166 mmol), DMF (3 mL), K₂CO₃ (46 mg, 0.33 mmol), and 1,3-bis(tert-butoxycarbonyl)guanidine (60 mg, 0.233 mmol) The reaction mixture was stirred at 50° C. for 2 hours. The reaction mixture was diluted with EtOAc (40 mL), washed with water, 10% LiCl, brine, dried over Na₂SO₄, concentrated and purified on silica gel. Elution with EtOAc/hexanes afforded the title compound (60 mg, 70% yield). ¹H NMR (300 MHz, CDCl₃) δ 9.38 (bs, 2H), 7.53 (d, J=9.0 Hz, 2H), 7.48 (d, J=9.0 Hz, 2H), 7.36 (m, 1H), 7.20 (d, J=6.0 Hz, 1H), 7.12 (s, 1H), 7.0 (d, J=6.0 Hz, 1H), 4.37 (t, 2H), 4.25 (t, 2H), 1.52-1.37 (3×9H).

### Example-14

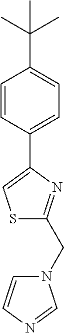 [SMILES: CC(C)(C)c1ccc(-c2csc(Cn3ccnc3)n2)cc1]

To a solution of 2-(bromomethyl)-4-(4-(tert-butyl)phenyl)thiazole 6b (10 mg, 0.0322 mmol) in 2.0 ml ACN was added excess imidazole (50 mg) and the reaction mixture was stirred at room temperature for 12 hours. The solvent was removed and the residue was purified eluting with CH₂Cl₂ to (10/89/1: MeOH/CHCl₃/ammonium hydroxide) afforded the desired compound (2.5 mg, 26% yield). LC/MS: 298 (M+H).

### Example-15

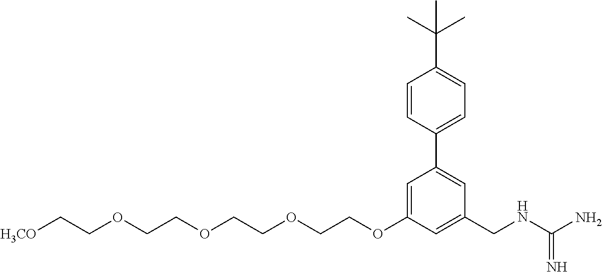 [SMILES: COCCOCCOCCOCCOc1cc(CNC(=N)N)cc(-c2ccc(C(C)(C)C)cc2)c1]

A 10-mL vial was added di-tert-butoxycarbonyl guanidine 15e (30 mg, 0.043 mmol), CH₂Cl₂ (2 mL), and TFA (1.5 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the desired compound (12 mg, 56% yield). ¹H NMR (300 MHz, CDCl₃) & 7.45 (m, 4H) 7.03 (s, 1H), 7.94 (s, 1H), 6.78 (s, 1R), 4.48 (s, 2H), 4.1-3.57 (m, 16H), 3.4 (s, 3H), 1.37 (s, 9H).

The required intermediates were prepared as follows:

a. Preparation of Compound

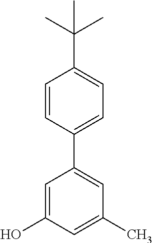 [SMILES: Cc1cc(O)cc(-c2ccc(C(C)(C)C)cc2)c1]

A 25-mL round bottom flask equipped with a magnetic stirrer, a condenser and a nitrogen in/outlet adapter was charged with 3-bromo-5-methylphenol (300 mg, 1.6 mmol), 4-tert-butylphenylboronic acid (428 mg, 2.1 mmol), water/dioxane (1 mL/3 ml), K₂CO₃ (441 mg, 3.2 mmol). The resulting solution was degassed for 5 minutes, then Pd(PPh₃)₄ (60 mg) was added. The reaction mixture was warmed to 100° C. and stirred for 3 hours. After being allowed to cool to room temperature, the reaction mixture was diluted with EtOAc (100 mL) and washed with saturated NaHCO₃, brine, dried over Na₂SO₄. The organic layer was concentrated under reduced pressure and purified on silica gel. Elution with EtOAc/hexanes solvent system afforded the desired compound (270 mg, 70% yield). ¹H NMR (300 MHz, CDCl₃) δ 7.54-7.46 (m, 4H), 7.01 (s, 1H), 6.89 (s, 1H), 6.65 (s, 1H), 2.39 (s, 3H), 1.38 (s, 9H).

b. Preparation of Compound

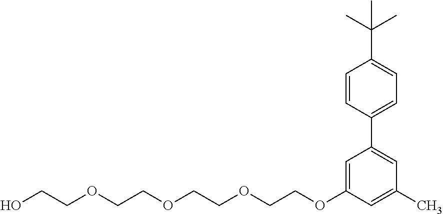 [SMILES: Cc1cc(OCCOCCOCCOCCO)cc(-c2ccc(C(C)(C)C)cc2)c1]

A 25-mL round bottom flask equipped with a magnetic stirrer was charged with the phenol intermediate 15a (270 mg, 1.12 mmol), DMF (2 mL), K₂CO₃ (345 mg, 2.5 mmol), and 2-(2-(2-(2-hydroxyethoxy)ethoxy)ethyl methanesulfonate (405 mg, 1.46 mmol) The reaction mixture was stirred at 50° C. for 12 hours. The reaction mixture was diluted with EtOAc, washed with water, 10% LiCl, brine, dried over Na₂SO₄, concentrated and purified on silica gel. Elution with 100% EtOAc afforded the desired compound product (300 mg, 64% yield). ¹H NMR (300 MHz, CDCl₃) δ 7.53-7.43 (m, 4H), 7.0 (s, 1H), 6.95 (s, 1H), 6.72 (s, 1H), 4.17 (m, 2H), 3.87 (m, 2H), 3.74-3.58 (m, 12H), 2.37 (s, 3H), 1.36 (s, 3H).

c. Preparation of Compound

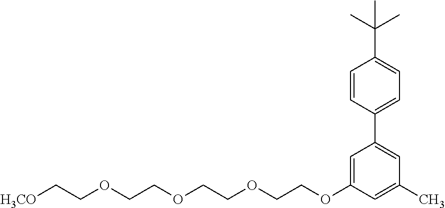 [SMILES: COCCOCCOCCOCCOc1cc(C)cc(-c2ccc(C(C)(C)C)cc2)c1]

To a solution of above alcohol 15b (80 mg, 0.192 mmol) in 3.0 mL THF at 0° C. was added NaH (40 mg) and the reaction mixture was stirred at room temperature for 1 hour. After 1 hour, MeI (0.3 mL) was added and the mixture was stirred one hour more after which THF was removed and was diluted with ethyl acetate. Careful washing with water and brine provided the crude product which was purified on an ISCO with silica using 50% EtOAC/hexane to yield the pure product (68 mg, 82% yield). ¹H NMR (300 MHz, CDCl₃) δ 7.50 (d, J=7.2 Hz, 2H), 7.43 (d, J=7.2 Hz, 2H), 6.99 (s, 1H), 6.94 (s, 1H), 6.71 (s, 1H), 4.16 (m, 2H), 3.87 (m, 2H), 3.73-3.51 (m, 12H), 3.37 (s, 3H), 2.37 (s, 3H), 1.36 (s, 9H).

d. Preparation of Compound

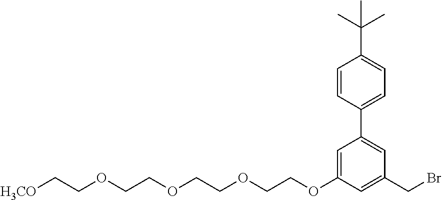 [SMILES: COCCOCCOCCOCCOc1cc(CBr)cc(-c2ccc(C(C)(C)C)cc2)c1]

A mixture of above compound 15c (68 mg, 0.158 mmol), NBS (40 mg, 0.205 mmol) in carbon tetrachloride (4.0 mL) was heated under light for 2 hours. The solids were filtered and the solvent was removed to give the crude product. Purification using 100% ethyl acetate afforded the product (40 mg, 80% yield) with some dibromo product which was used as crude for the next step.

e. Preparation of Compound

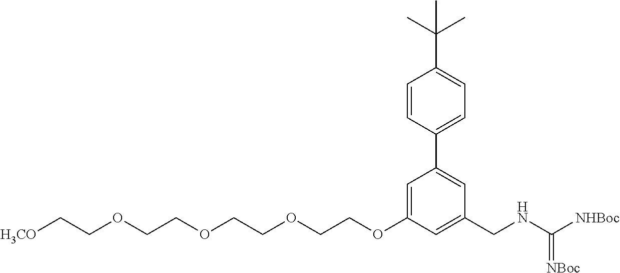

A 25-mL round bottom flask equipped with a magnetic stirrer and a condenser was charged with bromomethyl intermediate 15d (40 mg, 0.078 mmol), DMF (3 mL), K₂CO₃ (50 mg, 0.362 mmol), and 1,3-bis(tert-butoxycarbonyl)guanidine (41 mg, 0.115 mmol) The reaction mixture was stirred at 50° C. for 12 hours. The reaction mixture was diluted with EtOAc, washed with water, 10% LiCl, brine, dried over Na₂SO₄, concentrated to afford the crude desired compound (35 mg) which was used as such for the next step.

### Example 16

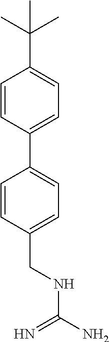 [SMILES: CC(C)(C)c1ccc(-c2ccc(CNC(=N)N)cc2)cc1]

To a 10-mL vial was added di-tert-butoxycarbonyl guanidine 16c (40 mg, 0.083 mmol), CH₂Cl₂ (0.5 mL), and TFA (0.5 mL). The sealed vial was stirred at 50° C. for 2 hours. The solvent was removed and the residue was purified on silica gel. Elution with CH₂Cl₂ to (10/89/1: MeOH/CHCl₃/ammonium hydroxide) afforded the title compound as a white solid (12 mg, 51% yield). ¹H NMR (300 MHz, CD₃OD) δ 7.64 (d, J=6.0 Hz, 2H), 7.55 (d, J=6.0 Hz, 2H), 7.47 (d, J=6.0 Hz, 2H), 7.39 (d, J=6.0 Hz, 2H), 4.40 (s, 2H), 1.35 (s, 9H).

The requisite intermediates were prepared as follows.

a. Preparation of Compound

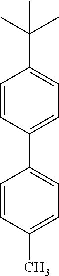 [SMILES: Cc1ccc(-c2ccc(C(C)(C)C)cc2)cc1]

A 50-mL round bottom flask equipped with a magnetic stirrer, a condenser and a nitrogen in/outlet adapter was charged with 4-bromotoluene (250 mg, 1.46 mmol), 4-tert-butylphenylboronic acid (390 mg, 2.2 mmol), DME (12.5 mL), Na₂CO₃ (5.0 ml)(2M). The resulting solution was degassed for 15 minutes, then Pd(PPh₃)₄ (150 mg) was added. The reaction mixture was warmed to 85° C. and stirred for 2 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with EtOAc/hexanes solvent system afforded the title compound (250 mg, 75% yield). ¹H NMR (300 MHz, CDCl₃) δ 7.53-7.44 (m, 6H), 7.24 (m, 2H), 2.39 (s, 3H), 1.36 (s, 9H).

b. Preparation of Compound

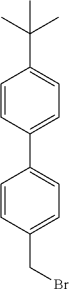 [SMILES: CC(C)(C)c1ccc(-c2ccc(CBr)cc2)cc1]

A mixture of the substituted toluene 16a (200 mg, 0.893 mmol), NBS (192 mg, 1.07 mmol) in carbon tetrachloride (2.0 mL) was heated under light for 2 hours. The solids were filtered and the solvent was removed to give the crude product. Purification using 10% ethyl acetate in hexane afforded the product (180 mg, 67% yield). ¹H NMR (300 MHz, CDCl₃) δ 7.55-7.46 (m, 8H), 4.55 (s, 2H), 1.36 (s, 9H).

c. Preparation of Compound

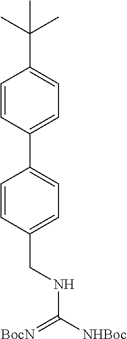

A 10-mL round bottom flask equipped with a magnetic stirrer, a condenser and a nitrogen in/outlet adapter was charged with the benzyl bromide 16b (37 mg, 0.13 mmol), DMF (1.0 mL), K₂CO₃ (37 mg, 0.265 mmol), and 1,3-bis(tert-butoxycarbonyl)guanidine (51 mg, 0.19 mmol) The reaction mixture was stirred at 50° C. for 2 hours. The reaction mixture was diluted with EtOAc, washed with water, 10% LiCl, brine, dried over Na₂SO₄, concentrated, and purified on silica gel. Elution with EtOAc/hexanes afforded the title compound as a white solid (40 mg, 64% yield). ¹H NMR (300 MHz, CDCl₃) δ 7.57-7.53 (m, 6H), 7.26 (m, 2H), 5.22 (s, 2H), 1.50 (s, 9H), 1.37 (s, 9H), 1.32 (s, 9H).

### Example 17

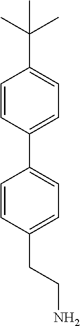 [SMILES: CC(C)(C)c1ccc(-c2ccc(CCN)cc2)cc1]

A THF solution of the nitrile 17a (22 mg, 0.098 mmol) was added to 0.48 mL LAH (1.0M) in THF at 0° C. The reaction mixture was then refluxed to 100° C. for 1 hour. After cooling to the room temperature, the normal LAH work-up procedure was followed. The solids were filtered and the solvent was removed and the residue was purified on silica gel. Elution with CH₂Cl₂ to (10/89/1: MeOH/CHCl₃/ammonium hydroxide) afforded the title compound. ¹H NMR (300 MHz, CD₃OD) δ 7.58-7.43 (m, 6H), 7.29 (d, J=6.0 Hz, 2H), 2.94 (m, 2H), 2.84 (m, 2H), 1.35 (s, 9H).

The requisite intermediate was prepared as follows:

a. Preparation of Compound

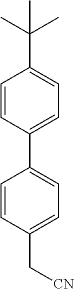 [SMILES: CC(C)(C)c1ccc(-c2ccc(CC#N)cc2)cc1]

A 25-mL round bottom flask equipped with a magnetic stirrer was charged with the benzyl bromide 16b (50 mg, 0.179 mmol), DMF (2.0 mL) followed by KCN (18 mg, 0.27 mmol). The reaction mixture was stirred at 50° C. for 2 hours. The reaction mixture was diluted with EtOAc, washed with water, 10% LiCl, brine, dried over Na₂SO₄, concentrated and purified on silica gel. Elution with EtOAc/hexanes afforded the desired compound (25 mg, 56% yield). ¹H NMR (300 MHz, CDCl₃) δ 7.60 (d, J=9.0 Hz, 2H), 7.54-7.46 (m, 4H), 7.38 (d, J=9.0 Hz, 2H), 3.79 (s, 2H), 1.37 (s, 9H).

### Example-18

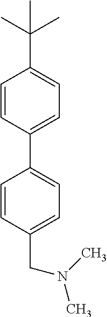 [SMILES: CN(C)Cc1ccc(-c2ccc(C(C)(C)C)cc2)cc1]

To a flask containing the benzyl bromide 16b (122 mg, 0.403 mmol) was added 1.0 mL dimethylamine (2M solution in THF). The reaction mixture was stirred at room temperature for 2 hours. The solvents were removed and the residue was purified on an ISCO with silica using 50% EtOAc in hexane to afford the desired product (55 mg, 64% yield). ¹H NMR (300 MHz, CDCl₃) δ 7.55 (d, J=9.0 Hz, 4H), 7.46 (d, J=6.0 Hz, 2H), 7.36 (d, J=6.0 Hz, 2H), 3.46 (s, 2H), 2.72 (s, 6H), 1.36 (s, 9H).

### Example-19

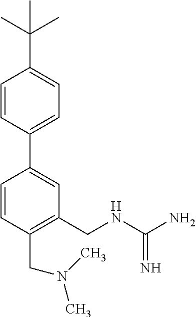 [SMILES: CN(C)Cc1ccc(-c2ccc(C(C)(C)C)cc2)cc1CNC(=N)N]

To a 10-mL vial was added di-tert-butoxycarbonyl guanidine 19a (12 mg, 0.022 mmol), CH₂Cl₂ (0.5 mL), and TFA (0.5 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the desired product (3.0 mg, 40% yield). ¹H NMR (300 MHz, CD₃OD) δ 7.64 (s, 1H), 7.55 (m, 5H), 7.30 (d, J=6.0 Hz, 1H), 4.48 (s, 2H), 3.57 (s, 2H), 2.29 (s, 6H), 1.36 (s, 9H).

The required intermediates were prepared as follows:

a. Preparation of Compound

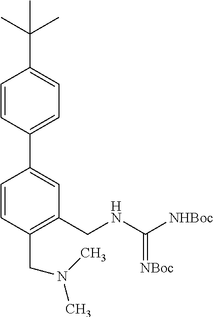

To a mixture of the alcohol 19b (13 mg, 0.044 mmol), PPh₃ (17 mg, 0.066 mmol) and 1,3-bis(tert-butoxycarbonyl)guanidine (23 mg, 0.088 mmol) in 1.0 mL toluene was added DIAD (0.013 ml) at room temperature. The reaction mixture was stirred for 16 hours at room temperature after which the solvent was removed to give the crude product. Purification on an ISCO with silica using 50% EtOAC in hexane afforded the desired product (12 mg, 51% yield). ¹H NMR (300 MHz, CDCl₃) δ 7.7-7.2 (m, 7H), 5.35 (s, 2H), 3.47 (s, 2H), 2.23 (s, 6H), 1.45 (s, 9H), 1.36 (s, 9H), 1.26 (s, 9H).

b. Preparation of Compound

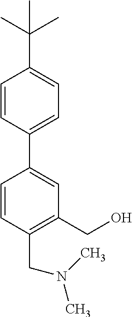 [SMILES: CN(C)Cc1ccc(-c2ccc(C(C)(C)C)cc2)cc1CO]

To a solution of 4-aminomethyl-4′-tert-butyl[1:1′]biphenyl 18 (49 mg, 0.184 mmol) in 1.0 mL dry ether under argon was added n-BuLI (0.090 ml, 2.5 M in hexane). The reaction mixture was stirred under argon for 10 hours at room temperature. After partition between ethyl acetate and water, the crude mixture was found to be mainly alcohol. The alcohol was used for the Mitsunobu step without further purification.

### Example-20

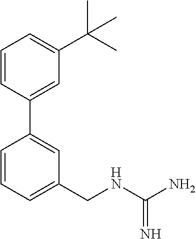 [SMILES: CC(C)(C)c1cccc(-c2cccc(CNC(=N)N)c2)c1]

To a 10-mL vial was added di-tert-butoxycarbonyl guanidine 20b (14 mg, 0.029 mmol), CH₂Cl₂ (1.0 mL), and TFA (1.0 mL). The sealed vial was stirred at room temperature overnight. The solvent was removed and the residue was purified on silica gel. Gradient elution with CH₂Cl₂ to (10/89/1: MeOH/CHCl₃/ammonium hydroxide) afforded the title compound (5 mg, 62% yield). ¹H NMR (300 MHz, CD₃OD) δ 7.64-7.41 (m, 8H), 4.50 (s, 2H), 1.40 (s, 9H).

The requisite intermediates were prepared as follows:

a. Preparation of Compound

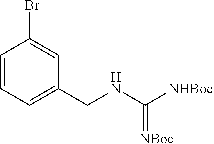

A 25 mL round bottom flask equipped with a magnetic stirrer was charged with 3-bromobenzyl bromide (500 mg, 2.0 mmol), DMF (4.0 mL), K₂CO₃ (553 mg, 4.0 mmol), and 1,3-bis(tert-butoxycarbonyl)guanidine (771 mg, 3.0 mmol) The reaction mixture was stirred at 50° C. for 2 hours. The reaction mixture was diluted with EtOAc, washed with water, 10% LiCl, brine, dried over Na₂SO₄, concentrated and purified on silica gel. Elution with EtOAc/hexanes afforded the title compound as a white solid (660 mg, 77% yield). ¹H NMR (300 MHz, CDCl₃) δ 9.4 (bs, 2H), 7.41 (d, J=8.1 Hz, 2H), 7.12 (d, J=7.8 Hz, 2H), 5.11 (s, 2H), 1.48 (s, 9H), 1.42 (s, 9H).

b. Preparation of Compound

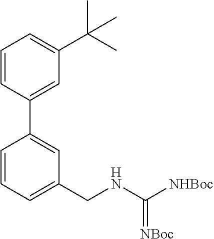

A 50-mL round bottom flask equipped with a magnetic stirrer, a condenser and a nitrogen in/outlet adapter was charged with aryl bromide 20a (50 mg, 0.117 mmol), 3-tert-butylphenylboronic acid (31 mg, 0.175 mmol), DME (1.5 mL), Na₂CO₃ (0.5 ml)(2M). The resulting solution was degassed for 15 minutes, then Pd(PPh₃)₄ (13 mg, 0.017 mmol) was added. The reaction mixture was warmed to 85° C. and stirred for 2 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with EtOAc/hexanes solvent system afforded the title compound (40 mg, 71% yield). ¹H NMR (300 MHz, CDCl₃) δ 7.60 (s, 1H), 7.52-7.39 (m, 5H), 7.28 (m, 2H), 5.27 (s, 2H), 1.51 (s, 9H), 1.39 (s, 9H), 1.38 (s, 9H).

### Example-21

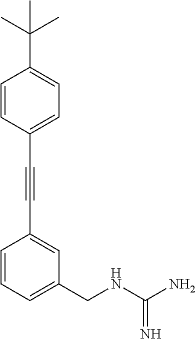 [SMILES: CC(C)(C)c1ccc(C#Cc2cccc(CNC(=N)N)c2)cc1]

To a 10-mL vial was added di-tert-butoxycarbonyl guanidine 21a (26 mg, 0.051 mmol), CH₂Cl₂ (1.0 mL), and TFA (1.0 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound as a white solid (11 mg, 71% yield). ¹H NMR (300 MHz, CD₃OD) δ 8.02 (d, J=8.4 Hz, 2H), 7.57 (d, J=8.4 Hz, 2H), 7.35 (m, 1H), 7.28 (m, 3H), 4.41 (s, 2H), 1.37 (s, 9H), LC/MS: 294.46 (M+H).

The requisite intermediate was prepared as follows:

a. Preparation of Compound

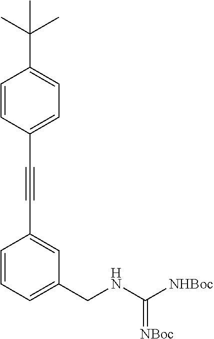

A solution of 20a (50 mg, 0.117 mmol), 1-(tert-butyl)-4-ethynylbenzene (0.063 mL, 0.35 mmol), CuI (3.2 mg, 0.007 mmol), and PPh₃ (8 mg, 0.029 mmol) in 2.0 mL TEA was degassed for 30 minutes. Then the catalyst Pd₂dba₃ (3.2 mg, 0.0035 mmol) was added and the resulting mixture was heated at 80° C. for 16 hours. After cooling to room temperature the solids were filtered and the solvents were removed to give the crude product. Purification in ISCO using 50% EtOAc/Hexane afforded the pure product (26 mg, 44% yield). ¹H NMR (300 MHz, CDCl₃) 9.4 (bs, 2H), 7.48-7.29 (m, 8H), 5.21 (s, 2H), 1.53 (s, 9H), 1.37 (s, 9H), 1.36 (s, 9H).

### Example-22

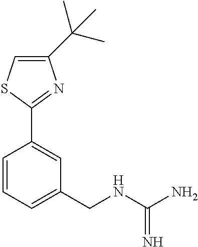 [SMILES: CC(C)(C)c1csc(-c2cccc(CNC(=N)N)c2)n1]

To a 10-mL vial was added di-tert-butoxycarbonyl guanidine 22b (20 mg, 0.04 mmol), CH₂Cl₂ (1.0 mL), and TFA (1.0 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound as a white solid (9 mg, 76% yield). ¹H NMR (300 MHz, CD₃OD) δ 7.95 (s, 1H), 7.88 (d, J=7.5 Hz, 1H), 7.49 (t, J=7.5 Hz, 1H), 7.40 (d, J=7.8 Hz, 1H), 7.16 (s, 1H), 4.49 (s, 2H), 1.39 (s, 9H).

The requisite intermediate was prepared as follows:

a. Preparation of Compound

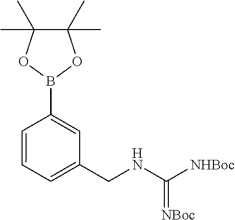

To a solution of aryl bromide 20a (230 mg, 0.537 mmol), in 5.0 mL dioxane was added KOAc (160 mg, 1.66 mmol), diborane (322 mg, 1.27 mmol) followed by Pd(dppf)Cl₂.DCM (88 mg). The mixture was heated at 80° C. for 16 h. The reaction mixture was cooled to room temperature, the solids were filtered off, the solvent was removed and the crude product (150 mg, 62% yield) was used without further purification.

b. Preparation of Compound

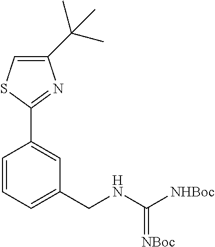

A 25-mL round bottom flask equipped with a magnetic stirrer, a condenser and a nitrogen in/outlet adapter was charged with 2-bromo-4-(tert-butyl)thiazole (0.013 ml, 0.084 mmol), above boronate ester 22a (50 mg, 0.105 mmol), DME (1.5 mL), Na₂CO₃ (0.4 ml)(2M). The resulting solution was degassed for 15 minutes, then Pd(PPh₃)₄ (13 mg, 0.017 mmol) was added. The reaction mixture was warmed to 85° C. and stirred for 2 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with EtOAc/hexanes solvent system afforded the title compound (23 mg). ¹H NMR (300 MHz, CDCl₃) δ 9.4 (bs, 2H), 7.96 (s, 1H), 7.85 (d, J=7.2 Hz, 1H), 7.40 (m, 2H), 6.90 (s, 1H), 5.25 (s, 2H), 1.53 (s, 9H), 1.44 (s, 9H), 1.41 (s, 9H).

### Example-23

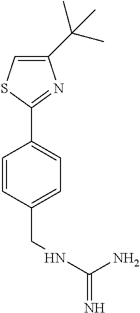 [SMILES: CC(C)(C)c1csc(-c2ccc(CNC(=N)N)cc2)n1]

To a 10-mL vial was added di-tert-butoxycarbonyl guanidine 23c (28 mg, 0.573 mmol), CH₂Cl₂ (1.0 mL), and TFA (1.0 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound as a white solid (13 mg, 79% yield). ¹H NMR (300 MHz, CD₃OD) δ 7.96 (d, J=8.4 Hz, 2H), 7.42 (d, J=8.4 Hz, 2H), 7.13 (s, 1H), 4.47 (s, 2H), 1.39 (s, 9H).

The requisite intermediates were prepared as follows:

a. Preparation of Compound

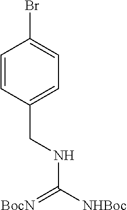

A 25 mL round bottom flask equipped with a magnetic stirrer was charged with 4-bromobenzyl bromide (870 mg, 2.82 mmol), DMF (4.0 mL), K₂CO₃ (583 mg, 4.24 mmol), and 1,3-bis(tert-butoxycarbonyl)guanidine (870 mg, 3.39 mmol) The reaction mixture was stirred at 80° C. for 2 hours. The reaction mixture was diluted with EtOAc, washed with water, 10% LiCl, brine, dried over Na₂SO₄, concentrated, and purified on silica gel. Elution with EtOAc/hexanes afforded the title compound as a white solid (600 mg, 50% yield).

b. Preparation of Compound

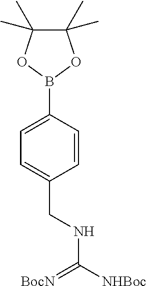

To a solution of the aryl bromide 23a (230 mg, 0.537 mmol), in 5.0 mL dioxane was added KOAc (160 mg, 1.66 mmol), diborane (322 mg, 1.27 mmol) followed by Pd(dppf)Cl₂.DCM (88 mg). The mixture was heated at 80° C. for 16 hours. The reaction mixture was cooled to room temperature, the solids were filtered off, the solvent was removed and the crude product was purified on an ISCO using silica and 10% EtOAc in hexane to afford the desired product (200 mg, 82% yield). ¹H NMR (300 MHz, CDCl₃) δ 9.49 (bs, 1H), 9.38 (bs, 1H), 7.75 (d, J=6.9 Hz, 2H), 7.24 (d, J=7.2 Hz, 2H), 5.22 (s, 2H), 1.53-1.37 (30H).

c. Preparation of Compound

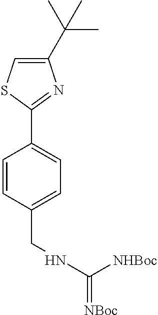

A 25-mL round bottom flask equipped with a magnetic stirrer, a condenser and a nitrogen in/outlet adapter was charged with 2-bromo-4-(tert-butyl)thiazole (0.02 ml), boronate ester 23b (50 mg), DME (1.5 mL), Na₂CO₃ (0.4 ml)(2M). The resulting solution was degassed for 15 minutes, then Pd(PPh₃)₄ (13 mg, 0.017 mmol) was added. The reaction mixture was warmed to 85° C. and stirred for 2 hours. After being allowed to cool to room temperature, the reaction mixture was diluted with EtOAc and washed with saturated NaHCO₃, then brine and dried over Na₂SO₄. The organic layer was concentrated under reduced pressure and purified on silica gel. Elution with EtOAc/hexanes solvent system afforded the title compound (28 mg) ¹H NMR (300 MHz, CDCl₃) δ 9.4 (bs, 2H), 7.93 (d, J=8.1 Hz, 2H), 7.31 (d, J=8.1 Hz, 2H), 6.89 (s, 1H), 5.23 (s, 2H), 1.52 (s, 9H), 1.42 (s, 9H), 1.38 (s, 9H).

### Example-24

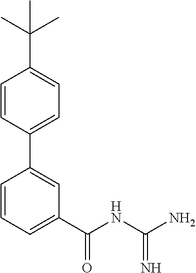 [SMILES: CC(C)(C)c1ccc(-c2cccc(C(=O)NC(=N)N)c2)cc1]

To a 10-mL vial was added di-tert-butoxycarbonyl guanidine 24b (20 mg, 0.04 mmol), CH₂Cl₂ (1.0 mL), and TFA (1.0 mL). The sealed vial was stirred at room temperature overnight. The solvent was removed and the residue was purified on silica gel. Elution with CH₂Cl₂ to (10/89/1: MeOH/CHCl₃/ammonium hydroxide) afforded the title compound as a white solid (6 mg, 50% yield). ¹H NMR (300 MHz, CD₃OD) δ 8.27 (s, 1H), 7.97 (d, J=7.8 Hz, 1H), 7.84 (d, J=8.1 Hz, 1H), 7.62 (d, J=8.7 Hz, 2H), 7.5 (m, 3H), 1.37 (s, 9H).

The requisite intermediates were prepared as follows:

a. Preparation of Compound

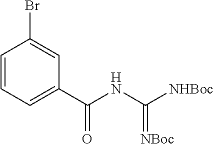

To a solution of 3-bromobenzoyl chloride (250 mg, 1.14 mmol) in 5.0 mL DCM was added Et₃N (0.3 mL) and 1,3-bis(tert-butoxycarbonyl)guanidine (322 mg, 1.25 mmol) and the reaction mixture was stirred for 12 hours at room temperature. The solvent was removed and the residue was purified on an ISCO with silica using 50% EtOAC in hexane to afford the desired product (124 mg, 25% yield). ¹H NMR (300 MHz, CDCl₃) δ 8.39 (s, 1H), 8.15 (d, J=8.1 Hz, 1H), 7.73 (d, J=8.4 Hz, 1H), 7.41 (t, J=7.8 Hz, 1H), 1.54 (s, 9H), 1.46 (s, 9H).

b. Preparation of Compound

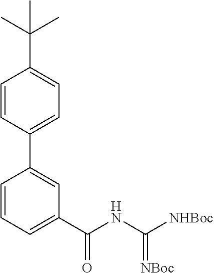

A 15-mL round bottom flask equipped with a magnetic stirrer, a condenser and a nitrogen in/outlet adapter was charged with bromo compound 24a (50 mg, 0.114 mmol), 4-tert-butylphenylboronic acid 30 mg, 0.17 mmol), DME (1.3 mL), Na₂CO₃ (0.5 ml)(2M). The resulting solution was degassed for 15 minutes, then Pd(PPh₃)₄ (13 mg, 0.11 mmol) was added. The reaction mixture was warmed to 85° C. and stirred for 1 hour. After being allowed to cool to room temperature, the reaction mixture was diluted with EtOAc and washed with saturated NaHCO₃, brine and dried over Na₂SO₄. The organic layer was concentrated under reduced pressure and purified on silica gel. Elution with EtOAc/hexanes solvent system afforded the title compound (23 mg, 41% yield). ¹H NMR (300 MHz, CD₃OD) δ 8.42 (t, J=1.8 Hz, 1H), 8.11 (d, J=7.8 Hz, 1H), 7.79 (d, J=7.8 Hz, 1H), 7.63 (d, J=6.0 Hz, 2H), 7.50 (m, 3H), 7.22 (d, J=6.3 Hz, 1H), 6.71 (d, J=8.7 Hz, 1H), 1.57 (s, 9H), 1.39 (s, 9H), 1.29 (s, 9H).

### Example-25

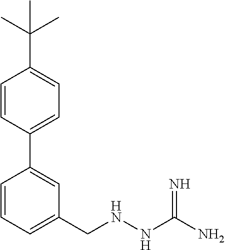 [SMILES: CC(C)(C)c1ccc(-c2cccc(CNNC(=N)N)c2)cc1]

To a solution of the amino guanidine 8 (75 mg, 0.252 mmol) in 5.0 mL ethanol was added 70 mg Pd/C. The reaction mixture was stirred under hydrogen balloon for 1 hour. The catalyst was filtered and the solvent was removed to give very pure product (60 mg, 80% yield). ¹H NMR (300 MHz, CD₃OD) δ 7.68-7.38 (m, 8H), 4.03 (s, 2H), 1.38 (s, 9H).

### Example-26

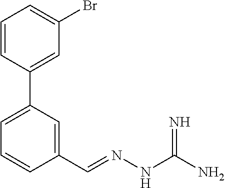 [SMILES: N=C(N)N/N=C/c1cccc(-c2cccc(Br)c2)c1]

A mixture of biaryl aldehyde 26a (50 mg, 0.19 mmol), aminoguanidine hydrochloride (26 mg, 0.23 mmol) in 1 mL EtOH was refluxed overnight. The resulting solid was filtered, washed with ether and was dried to produce the desired compound in high purity (40 mg, 66% yield). ¹H NMR (300 MHz, CD₃OD) δ 8.15 (s, 1H), 8.06 (s, 1H), 7.81 (s, 1H), 7.75 (d, J=7.8 Hz, 1H), 7.68-7.61 (m, 2H), 7.51 (t, J=6.0 Hz, 2H), 7.35 (t, J=8.1 Hz, 1H),

The requisite intermediate was prepared as follows:

a. Preparation of compound

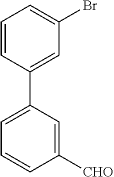 [SMILES: O=Cc1cccc(-c2cccc(Br)c2)c1]

A 50-mL round bottom flask equipped with a magnetic stirrer, a condenser and a nitrogen in/outlet adapter was charged with 1,3-dibromobenzene (600 mg, 2.5 mmol), 3-formylphenylboronic acid (128 mg), DME (10.0 mL), Na₂CO₃ (3.8 ml)(2M). The resulting solution was degassed for 15 minutes, then Pd(PPh₃)₄ (90 mg) was added. The reaction mixture was warmed to 85° C. and stirred for 2 hours. After being allowed to cool to room temperature, the reaction mixture was diluted with EtOAc and washed with saturated NaHCO₃, then brine and dried over Na₂SO₄. The organic layer was concentrated under reduced pressure and purified on silica gel. Elution with EtOAc/hexanes solvent system afforded the title compound (139 mg, 21% yield). ¹H NMR (300 MHz, CDCl₃) δ 8.07 (s, 1H), 7.9-7.7 (m, 3H), 7.63 (m, 2H), 7.54 (m, 2H), 7.35 (t, J=6.0 Hz, 1H).

### Example-27

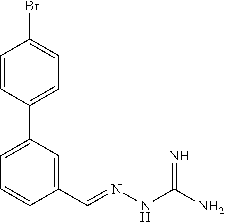 [SMILES: N=C(N)N/N=C/c1cccc(-c2ccc(Br)cc2)c1]

A mixture of biaryl aldehyde 27a (50 mg, 0.19 mmol), aminoguanidine hydrochloride (26 mg, 0.23 mmol) in 1 ml EtOH was refluxed overnight. The resulting solid was filtered, washed with ether and was dried to produce the desired compound in high purity (43 mg, 71% yield). ¹H NMR (300 MHz, CD₃OD) δ 8.18 (s, 1H), 8.10 (s, 1H), 7.77 (d, J=6.0 Hz, 1H), 7.70 (d, J=6.0 Hz, 1H), 7.62 (m, 4H), 7.53 (t, J=7.8 Hz, 1H).

The following intermediate was prepared as follows:

a. Preparation of Compound

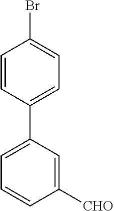 [SMILES: O=Cc1cccc(-c2ccc(Br)cc2)c1]

A 50-mL round bottom flask equipped with a magnetic stirrer, a condenser and a nitrogen in/outlet adapter was charged with 1,4-dibromobenzene (600 mg, 2.5 mmol), 3-formylphenylboronic acid (128 mg), DME (10 mL), Na₂CO₃ (3.8 ml, 2M). The resulting solution was degassed for 15 minutes, then Pd(PPh₃)₄ (90 mg) was added. The reaction mixture was warmed to 85° C. and stirred for 2 hours. After being allowed to cool to room temperature, the reaction mixture was diluted with EtOAc and washed with saturated NaHCO₃, then brine and dried over Na₂SO₄. The organic layer was concentrated under reduced pressure and purified on silica gel. Elution with EtOAc/hexanes solvent system afforded the title compound (215 mg, 32% yield). ¹H NMR (300 MHz, CDCl₃) δ 10.09 (s, 1H), 8.07 (s, 1H), 7.89-7.81 (m, 2H), 7.60 (d, J=8.4 Hz, 2H), 7.50 (d, J=8.4 Hz, 2H), 7.34 (d, J=6.0 Hz, 1H).

### Example-28

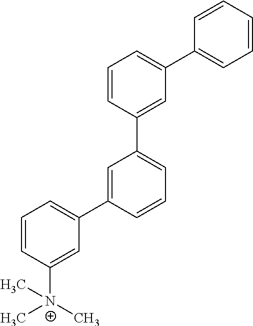 [SMILES: C[N+](C)(C)c1cccc(-c2cccc(-c3cccc(-c4ccccc4)c3)c2)c1]

A solution of the starting material N,N-dimethylaniline 28b (15 mg, 0.04 mmol) in iodomethane (1.0 mL) was stirred in a sealed 2-dram vial at 80° C. overnight. After being allowed to cool to room temperature, Et₂O was added to the reaction mixture. The solid was collected by filtration to afford the title compound (18 mg, 90%) as light yellow solid. ¹H NMR (CDCl₃, 400 MHz) δ 7.36-7.96 (m, 16H), 4.14 (s, 9H).

The requisite intermediates were prepared as follows:

a. Preparation of Compound

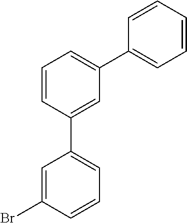 [SMILES: Brc1cccc(-c2cccc(-c3ccccc3)c2)c1]

A 200-mL round bottom flask equipped with a magnetic stirrer, a condenser and a nitrogen in/outlet adapter was charged with 1-bromo-3-iodobenzene (2.85 g, 10.1 mmol), 3-biphenylboronic acid (1.0 g, 5.05 mmol), water/dioxane (10 mL/40 ml), K₂CO₃ (1.4 g, 10 mmol). The resulting solution was degassed for 5 minutes, then Pd(PPh₃)₄ (192 mg, 0.25 mmol) was added. The reaction mixture was heated to 80° C. for 4 hours. After being allowed to cool to room temperature, the reaction mixture was diluted with EtOAc (100 mL) and washed with saturated NaHCO₃ (40 mL), then brine (40 mL) and dried over Na₂SO₄. The organic layer was concentrated under reduced pressure and purified on silica gel. Elution with hexanes afforded the title compound (782 mg, 50%) colorless oil. ¹H NMR (CDCl₃, 300 MHz) δ 7.76-7.80 (m, 2H), 7.31-7.67 (m, 11H).

b. Preparation of Compound

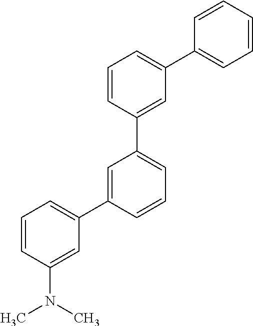 [SMILES: CN(C)c1cccc(-c2cccc(-c3cccc(-c4ccccc4)c3)c2)c1]

A 25-mL round bottom flask equipped with a magnetic stirrer, a condenser and a nitrogen in/outlet adapter was charged with 3-bromo-1,1′:3′,1″-terphenyl 28a (50 mg, 0.16 mmol), 3-(dimethylamino)phenylboronic acid (38 mg, 0.23 mmol), water/dioxane (1 mL/4 ml), K₂CO₃ (45 mg, 0.32 mmol). The resulting solution was degassed for 5 minutes, then Pd(PPh₃)₄ (9 mg, 0.008 mmol) was added. The reaction mixture was heated to 90° C. for 2 hours. After being allowed to cool to room temperature, the reaction mixture was diluted with EtOAc and washed with saturated NaHCO₃, brine, dried over Na₂SO₄. The organic layer was concentrated under reduced pressure and purified on silica gel. Elution with 5% EtOAc/hexanes afforded the title compound (29 mg, 51%) as a white solid. ¹H NMR (CDCl₃, 400 MHz) δ 7.78 (s, 2H), 7.24-7.60 (m, 13H), 6.90-6.94 (m, 2H), 6.69-6.71 (m, 1H), 2.90 (s, 6H).

### Example-29

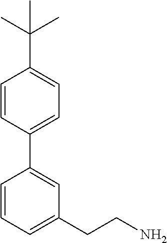 [SMILES: CC(C)(C)c1ccc(-c2cccc(CCN)c2)cc1]

A 10-mL vial was added tert-butyl (2-(4′-(tert-butyl)-[1,1′-biphenyl]-3-yl)ethyl)carbamate 29b (100 mg, 0.03 mmol), CH₂Cl₂ (1 mL), and TFA (1 mL). The sealed vial was stirred at 50° C. for 1 hour. The solvent was removed and the residue was purified on silica gel. Elution with 5% MeOH/EtOAc afforded the title compound (45 mg, 63%) as colorless oil. ¹H NMR (CDCl₃, 400 MHz) δ 7.45 (d, 2H, J=8.5 Hz), 7.38 (d, 2H, J=8.5 Hz), 7.35 (m, 2H), 7.29 (t, J=4.6 Hz, 1H), 7.09 (d, J=7.5 Hz, 1H), 2.94 (t, J=6.9 Hz, 2H), 2.74 (t, J=6.8 Hz, 2H), 1.29 (s, 9H).

The requisite intermediate was prepared as follows:

a. Preparation of Compound

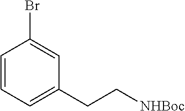 [SMILES: CC(C)(C)OC(=O)NCCc1cccc(Br)c1]

Prepared by the method described by Herth, Matthias M., et al., Bioorganic & Medicinal Chemistry, 2012, 20, 4574-4581.

b. Preparation of Compound

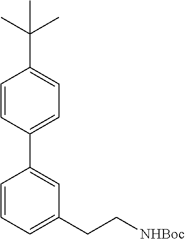 [SMILES: CC(C)(C)OC(=O)NCCc1cccc(-c2ccc(C(C)(C)C)cc2)c1]

A 25-mL round bottom flask equipped with a magnetic stirrer, a condenser and a nitrogen in/outlet adapter was charged with tert-butyl 3-bromophenethylcarbamate 29a (170 mg, 0.57 mmol), (4-(tert-butyl)phenyl)boronic acid (148 mg, 0.83 mmol), water/dioxane (1 mL/4 ml), K₂CO₃ (157 mg, 1.14 mmol). The resulting solution was degassed for 5 minutes, then Pd(PPh₃)₄ (12 mg, 0.01 mmol) was added. The reaction mixture was heated to 100° C. for 2 hours. After cooling to room temperature, the reaction mixture was diluted with EtOAc and washed with saturated NaHCO₃, brine, dried over Na₂SO₄. The organic layer was concentrated and purified on silica gel. Elution with 5% EtOAc/hexanes afforded the title compound (180 mg, 90%) as colorless oil. ¹H NMR (CDCl₃, 400 MHz) δ 7.54 (d, J=8.5 Hz, 2H), 7.48 (m, 3H), 7.43 (s, 1H), 7.39 (t, J=7.6 Hz, 1H), 7.18 (d, J=7.5 Hz, 1H), 4.58 (bs, 1H), 3.45 (m, 2H), 2.88 (t, J=6.9 Hz, 2H), 1.46 (s, 9H), 1.39 (s, 9H).

### Example-30

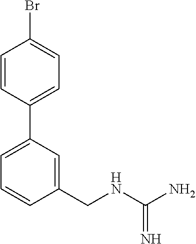 [SMILES: N=C(N)NCc1cccc(-c2ccc(Br)cc2)c1]

A 10-mL vial was added 30b (30 mg, 0.06 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (10 mg, 55%) as a white solid. ¹H NMR (DMSO-d₆, 300 MHz) δ 7.98 (s, 1H), 7.70-7.61 (m, 5H), 7.49 (t, J=8.4 Hz, 1H), 7.32 (d, J=7.5 Hz, 1H), 4.43 (s, 2H).

The requisite intermediates were prepared as follows:

a. Preparation of Compound

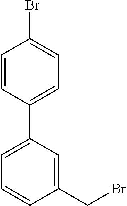 [SMILES: BrCc1cccc(-c2ccc(Br)cc2)c1]

A 100-mL round bottom flask equipped with a magnetic stirrer, a condenser and a nitrogen in/outlet adapter was charged with 4′-bromo-3-methyl-1,1′-biphenyl (580 g, 2.35 mmol), CCl₄ (10 mL), and NBS (460 mg, 2.59 mmol). The reaction mixture was refluxed for 5 hours by heating with bulb light. The starting material was consumed by TLC. After cooling to room temperature, the reaction mixture was added hexanes (60 mL). The solid was removed by filtration and the filtrate was concentrated under reduced pressure and purified on silica gel. Elution with 5% hexanes afforded the title compound (550 mg, 72%) as colorless oil. ¹H NMR (CDCl₃, 300 MHz) δ 7.55-7.59 (m, 4H), 7.39-7.49 (m, 6H), 4.54 (s, 2H).

b. Preparation of Compound

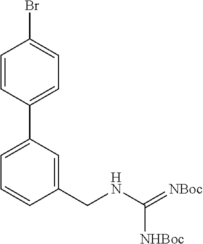

A 25-mL round bottom flask equipped with a magnetic stirrer under nitrogen was charged with 30a (30 mg, 0.06 mmol), DMF (1 mL), K₂CO₃ (16 mg, 0.12 mmol), and 1,3-bis(tert-butoxycarbonyl)guanidine (20 mg, 0.07 mmol) The reaction mixture was stirred at room temperature for 12 hours. The reaction mixture was diluted with EtOAc (60 mL), washed with water (20 mL), 10% LiCl (20 mL), brine (20 mL), dried over Na₂SO₄, concentrated, and purified on silica gel. Elution with 5% EtOAc/hexanes afforded the title compound (35 mg, 85%) as a white solid. ¹H NMR (CDCl₃, 300 MHz) δ 9.37 (broad s, 1H), 9.31 (broad s, 1H), 7.53-7.56 (m, 3H), 7.41-7.45 (m, 3H), 7.36 (t, 1H, J=5.70 Hz), 7.24 (m, 1H), 5.22 (s, 2H), 1.48 (s, 9H), 1.35 (s, 9H).

### Example 31

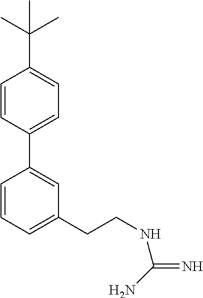 [SMILES: CC(C)(C)c1ccc(-c2cccc(CCNC(=N)N)c2)cc1]

A 10-mL vial was added 31a (30 mg, 0.06 mmol), CH₂Cl₂ (1 mL), and TFA (1 mL). The sealed vial was stirred at room temperature for 1 hour. The solvent was removed and the residue was purified on silica gel. Elution with CH₂Cl₂ to (10/89/1: MeOH/CHCl₃/ammonium hydroxide) afforded the title compound (15 mg, 83%) as a white solid. ¹H NMR (CDCl₃, 300 MHz) δ 7.84 (broad s, 1H), 7.33-7.52 (m, 7H), 7.17 (d, 1H, J=7.5 Hz), 3.34 (q, 2H, J=6.30 Hz), 2.92 (t, 2H, J=6.60 Hz), 1.34 (s, 9H). LCMS: 296.00

The requisite intermediate was prepared as follows:

a. Preparation of Compound

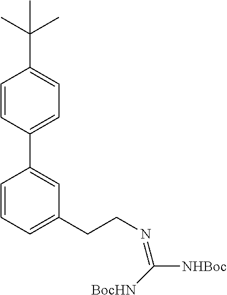 [SMILES: CC(C)(C)OC(=O)NC(=NCCc1cccc(-c2ccc(C(C)(C)C)cc2)c1)NC(=O)OC(C)(C)C]

A 25-mL round bottom flask equipped with a magnetic stirrer under nitrogen was charged with 2-(4′-(tert-butyl)-[1,1′-biphenyl]-3-yl)ethanamine 29 (30 mg, 0.11 mmol), CH₂Cl₂ (1 mL), and triethylamine (17 μL, 0.13 mmol), and di-tert-butyl ((trifluoromethylsulfonamido)methylene)-dicarbamate (50 mg, 0.13 mmol). The resulting reaction mixture was stirred at room temperature overnight. The reaction mixture was diluted with CH₂Cl₂ and washed with saturated NaHCO₃, brine, dried over Na₂SO₄, concentrated and purified using silica gel. Elution with 10% EtOAc/hexanes afforded the title compound (37 mg, 62%) as white solid. ¹H NMR (CDCl₃, 300 MHz) δ 8.44 (broad s, 1H), 7.52-7.55 (m, 2H), 7.42-7.46 (m, 4H), 7.38 (t, 1H, J=7.8 Hz), 7.17 (d, 1H, J=7.5 Hz), 3.71 (q, 2H, J=5.40 Hz), 2.92 (t, 2H, J=6.40 Hz), 1.49 (s, 9H), 1.43 (s, 9H), 1.36 (s, 9H).

### Example 32

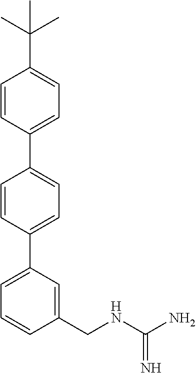 [SMILES: CC(C)(C)c1ccc(-c2ccc(-c3cccc(CNC(=N)N)c3)cc2)cc1]

A 10-mL vial was added 32a (10 mg, 0.02 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (4.5 mg, 70%) as a white solid. ¹H NMR (CDCl₃, 300 MHz) δ 8.59 (broad s, 1H), 7.39-7.67 (m, 11H), 7.22 (d, 1H, J=7.8 Hz), 4.35 (s, 2H), 1.36 (s, 9H).

The requisite intermediates were prepared as follows:

a. Preparation of Compound

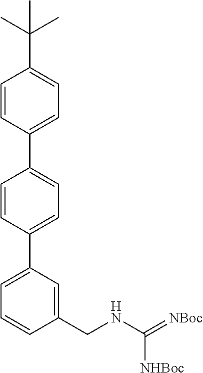

A 25-mL round bottom flask equipped with a magnetic stirrer, a condenser and a nitrogen in/outlet adapter was charged with di-tert-butyl ((((4′-bromo-[1,1′-biphenyl]-3-yl)methyl)amino)-methylene)dicarbamate 30b (30 mg, 0.06 mmol), (4-(tert-butyl)phenyl)boronic acid (16 mg, 0.09 mmol), water/dioxane (1 mL/3 ml), K₂CO₃ (16 mg, 0.12 mmol). The resulting solution was degassed for 5 minutes, then Pd(PPh₃)₄ (5 mg, 0.004 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated under reduced pressure and purified on silica gel. Elution with 10% EtOAc/hexanes afforded the title compound (16 mg, 48%) as a white foam. ¹H NMR (CDCl₃, 300 MHz) δ 8.59 (broad s, 1H), 7.35-7.65 (m, 11H), 7.22 (d, 1H, J=7.8 Hz), 5.28 (s, 2H), 1.51 (s, 9H), 1.39 (s, 9H), 1.36 (s, 9H).

### Example-33

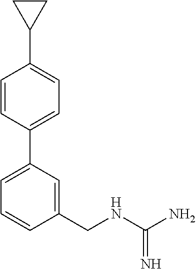 [SMILES: N=C(N)NCc1cccc(-c2ccc(C3CC3)cc2)c1]

A 2-dram vial was added diboc guanidine compound 33a (15 mg, 0.03 mmol), CH₂Cl₂ (1 mL), and TFA (1 mL). The sealed vial was stirred at room temperature for 12 hours. The solvent was removed and the residue was purified on silica gel. Elution with CH₂Cl₂ to (10/89/1: MeOH/CHCl₃/ammonium hydroxide) afforded the title compound (6.0 mg, 70%) as a white foam. ¹H NMR (CDCl₃, 300 MHz) δ 8.51 (s, 1H), 7.52-7.37 (m, 4H), 7.20-7.11 (m, 3H), 4.33 (d, J=5.4 Hz, 2H), 1.93 (m, 1H), 1.25-0.77 (m, 4H).

The requisite intermediates were prepared as follows:

a. Preparation of Compound

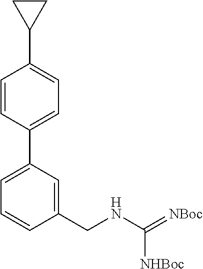

A 100-mL round bottom flask equipped with a magnetic stirrer, a condenser and a nitrogen in/outlet adapter was charged with di-tert-butyl ((((4′-bromo-[1,1′-biphenyl]-3-yl)methyl)amino)methylene)dicarbamate 30b (51 mg, 0.1 mmol), cyclopropylboronic acid (17 mg, 0.2 mmol), water/toluene (1 mL/20 ml), K₃PO₄ (64 mg, 0.5 mmol), and tricyclohexylphosphine (2.8 mg, 0.01 mmol). The resulting solution was degassed for 5 minutes, then Pd(OAc)₂ (5 mg, 0.003 mmol) was added. The reaction mixture was heated to 110° C. for 5 hours. After cooling to room temperature, the reaction mixture was diluted with EtOAc (30 mL) and washed with saturated NaHCO₃ (10 mL), brine (10 mL), dried over Na₂SO₄. The organic layer was concentrated and purified on silica gel. Elution with 2% EtOAc/hexanes afforded the title compound (16 mg, 34%) as a white foam. ¹H NMR (CDCl₃, 300 MHz) δ 9.5 (bs, 1H), 9.45 (bs, 1H), 7.6-7.15 (m, 8H), 5.24 (s, 2H), 1.98 (m, 1H), 1.45 (s, 9H), 1.36 (s, 9H), 1.02 (m, 2H), 0.8 (m, 2H).

### Example-34

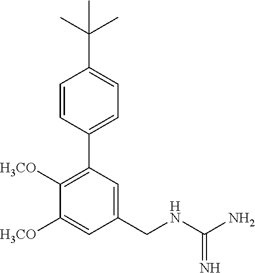 [SMILES: COc1cc(CNC(=N)N)cc(-c2ccc(C(C)(C)C)cc2)c1OC]

A 10-mL vial was added 34c (35 mg, 0.05 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (16 mg, 67%) as a white solid. ¹H NMR (CDCl₃, 300 MHz) δ 8.42 (broad s, 1H), 7.41 (s, 4H), 6.82 (d, 1H, J=1.20 Hz), 6.80 (d, 1H, J=1.20 Hz), 4.24 (d, 2H, J=4.5 Hz), 3.86 (s, 3H), 3.58 (s, 3H), 1.33 (s, 9H).

The requisite intermediates were prepared as follows:

a. Preparation of Compound

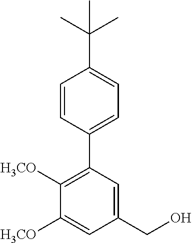 [SMILES: COc1cc(CO)cc(-c2ccc(C(C)(C)C)cc2)c1OC]

A 100-mL round bottom flask equipped with a magnetic stirrer, a condenser and a nitrogen in/outlet adapter was charged with (3-bromo-4,5-dimethoxyphenyl)methanol (520 mg, 2.41 mmol), (4-(tert-butyl)phenyl)boronic acid (715 mg, 3.61 mmol), water/dioxane (5 mL/15 ml), K₂CO₃ (665 mg, 4.82 mmol). The resulting solution was degassed for 5 minutes, then Pd(PPh₃)₄ (80 mg, 0.07 mmol) was added. The reaction mixture was heated to 100° C. for 2 hours. After cooling to room temperature, the reaction mixture was diluted with EtOAc and washed with saturated NaHCO₃, brine, dried over Na₂SO₄. The organic layer was concentrated and purified on silica gel. Elution with 10% EtOAc/hexanes afforded the title compound (586 mg, 81%) as colorless oil. ¹H NMR (CDCl₃, 300 MHz) δ 7.48 (d, 2H, J=8.60 Hz), 7.42 (d, 2H, J=8.60 Hz), 6.95 (d, 1H, J=1.20 Hz), 6.93 (d, 1H, J=1.20 Hz), 4.68 (s, 2H), 3.91 (s, 3H), 3.59 (s, 3H), 1.35 (s, 9H).

b. Preparation of Compound

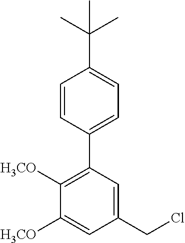 [SMILES: COc1cc(CCl)cc(-c2ccc(C(C)(C)C)cc2)c1OC]

A 25-mL round bottom flask equipped with a magnetic stirrer under nitrogen was charged with 34a (144 mg, 0.48 mmol), CH₂Cl₂ (5 mL), and triethylamine (0.14 mL, 0.96 mmol). After cooling to 0° C., methanesulfonyl chloride (0.08 mL, 0.96 mmol) was added via a syringe. The resulting reaction mixture was stirred at room temperature overnight. The reaction mixture was diluted with CH₂Cl₂ and washed with saturated NaHCO₃, brine, dried over Na₂SO₄, concentrated to afford the title compound (95 mg, 62%) as colorless oil which was used without further purification.

c. Preparation of Compound

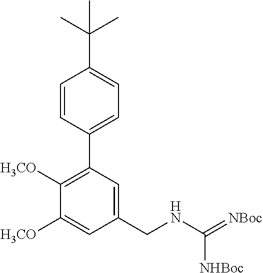

A 50-mL round bottom flask equipped with a magnetic stirrer, a condenser and a nitrogen in/outlet adapter was charged with 34b (19 mg, 0.06 mmol), DMF (1 mL), K₂CO₃ (16 mg, 0.12 mmol), and 1,3-bis(tert-butoxycarbonyl)guanidine (20 mg, 0.07 mmol) The reaction mixture was stirred at room temperature for 12 hours. The reaction mixture was diluted with EtOAc (60 mL), washed with water (20 mL), 10% LiCl (20 mL), brine (20 mL), dried over Na₂SO₄, concentrated, and purified on silica gel. Elution with 5% EtOAc/hexanes afforded the title compound (30 mg, 92%) as a white solid. ¹H NMR (CDCl₃, 300 MHz) δ 9.43 (broad s, 1H), 9.31 (broad s, 1H), 7.40-7.51 (m, 4H), 7.01 (d, 1H, J=1.2 Hz), 7.01 (d, 1H, J=1.20 Hz), 6.94 (d, 1H, J=1.20 Hz), 5.12 (s, 2H), 3.88 (s, 3H), 3.57 (s, 3H), 1.49 (s, 9H), 1.39 (s, 9H), 1.35 (s, 9H).

### Example-35

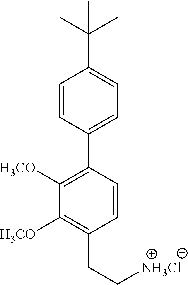

A 25-mL round bottom flask equipped with a magnetic stirrer, a condenser and a nitrogen in/outlet adapter was charged with N-(2-(4′-(tert-butyl)-2,3-dimethoxy-[1,1′-biphenyl]-4-yl)ethyl)acetamide (60 mg, 0.17 mmol), 6 N HCl (1.5 mL), MeOH (1.5 mL). The reaction mixture was heated to 100° C. for 12 hours. After cooling to room temperature, the reaction mixture was water. The solid was collected by filtration. After drying, there was obtained the title compound (55 mg, 93%) as a white solid. ¹H NMR (DMSO-d₆, 300 MHz) δ 8.09 (s, 2H), 7.43 (m, 4H), 7.05 (s, 2H), 3.84 (s, 3H), 3.57 (s, 3H), 3.06 (m, 2H), 2.93 (m, 2H), 1.31 (s, 9H).

### Example-36

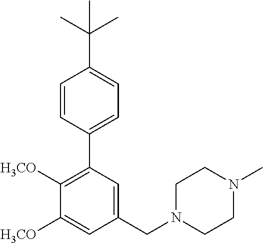 [SMILES: COc1cc(CN2CCN(C)CC2)cc(-c2ccc(C(C)(C)C)cc2)c1OC]

A 2-dram vial was added 4′-(tert-butyl)-5-(chloromethyl)-2,3-dimethoxy-1,1′-biphenyl 34b (20 mg, 0.06 mmol), CH₃CN (1 mL), 1-methylpiperazine (7 mg, 0.07 mmol). The sealed vial was heated to 60° C. for 2 hours. After cooling to room temperature, the solvent was removed and the residue was purified on silica gel. Elution with CH₂Cl₂ to (10/89/1: MeOH/CHCl₃/ammonium hydroxide) afforded the title compound (21 mg, 87%) as a write solid. ¹H NMR (CDCl₃, 300 MHz) δ 7.48 (d, 2H, J=7.8 Hz), 7.42 (d, 2H, J=8.7 Hz), 6.90 (s, 1H), 6.88 (s, 1H), 3.90 (s, 3H), 3.58 (s, 3H), 3.51 (s, 2H), 2.59 (broad s, 8H), 2.38 (s, 3H), 1.35 (s, 9H).

### Example-37

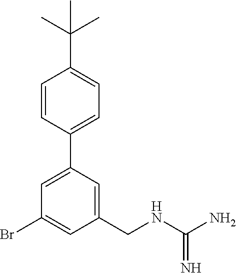 [SMILES: CC(C)(C)c1ccc(-c2cc(Br)cc(CNC(=N)N)c2)cc1]

A 10-mL vial was added diboc guanidine 37d (30 mg, 0.05 mmol), CH₂Cl₂ (1 mL), and TFA (1 mL) The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (12 mg, 63%) as a white solid. ¹H NMR (CDCl₃, 300 MHz) δ 8.68 (broad s, 1H), 7.66 (s, 1H), 7.46 (s, 4H), 7.39 (s, 1H), 7.34 (s, 1H), 4.34 (d, 2H, J=5.4 Hz), 1.34 (s, 9H).

The requisite intermediates were prepared as follows:

a. Preparation of Compound

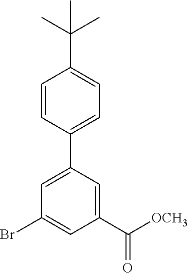 [SMILES: COC(=O)c1cc(Br)cc(-c2ccc(C(C)(C)C)cc2)c1]

A 500-mL round bottom flask equipped with a magnetic stirrer, a condenser and a nitrogen in/outlet adapter was charged with methyl 3-bromo-5-iodobenzoate (2.83 g, 8.3 mmol), (4-(tert-butyl)phenyl)boronic acid (1.64 g, 8.3 mmol), water/dioxane (15 mL/60 ml), K₂CO₃ (2.3 g, 16.6 mmol). The resulting solution was degassed for 5 minutes, then Pd(PPh₃)₄ (340 mg, 0.29 mmol) was added. The reaction mixture was heated to 60° C. for 24 hours. After being allowed to cool to room temperature, the reaction mixture was diluted with EtOAc (150 mL) and washed with saturated NaHCO₃ (50 mL), brine (50 mL), dried over Na₂SO₄. The organic layer was concentrated and purified on silica gel. Elution with hexanes afforded the title compound (1.69 g, 59%) colorless oil. ¹H NMR (CDCl₃, 300 MHz) δ 8.18 (s, 1H), 8.11 (s, 1H), 7.90 (s, 1H), 7.55-7.46 (m, 4H), 3.95 (s, 3H), 1.36 (s, 9H).

b. Preparation of Compound

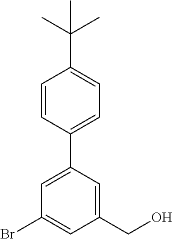 [SMILES: CC(C)(C)c1ccc(-c2cc(Br)cc(CO)c2)cc1]

A 50-mL round bottom flask equipped with a magnetic stirrer, and a condenser under nitrogen was charged with methyl 5-bromo-4′-(tert-butyl)-[1,1′-biphenyl]-3-carboxylate 37a (1.0 g, 2.90 mmol), THF (15 mL). After being allowed to cool to 0° C., LiBH₄ (63 mg, 2.9 mmol) was added in one portion. The resulting reaction mixture was warmed to room temperature then heated to 85° C. for 3 hours. After being allowed to cool to room temperature, acetone (1 ml) was added to the reaction mixture and stirred for 10 minutes. The solvent was removed and the residue was diluted with ethyl acetate. Washing with 1 N HCl, followed by saturated NaHCO₃, brine, the organic layers were dried over Na₂SO₄, concentrated and purified with silica gel. Elution with 20% EtOAc/hexanes afforded the title compound (740 mg, 80%) as a white solid. ¹H NMR (CDCl₃, 300 MHz) δ (s, 1H), 7.48-7.43 (m, 6H), 4.72 (s, 2H), 1.35 (s, 9H).

c. Preparation of Compound

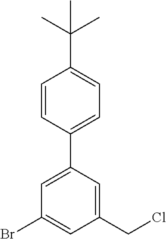 [SMILES: CC(C)(C)c1ccc(-c2cc(Br)cc(CCl)c2)cc1]

A 50-mL round bottom flask equipped with a magnetic stirrer under nitrogen was charged with (5-bromo-4′-(tert-butyl)-[1,1′-biphenyl]-3-yl)methanol 37b (740 mg, 2.23 mmol), CH₂Cl₂ (10 mL), and triethylamine (0.48 mL, 3.48 mmol). After being allowed to cool to 0° C., methanesulfonyl chloride (0.23 mL, 3.0 mmol) was added via a syringe. The resulting reaction mixture was stirred at room temperature overnight. The reaction mixture was diluted with CH₂Cl₂ (30 mL) and washed with saturated NaHCO₃ (15 ml), brine (15 mL), dried over Na₂SO₄, concentrated to purified with silica gel and elution with CH₂Cl₂ to afford the title compound (702 mg, 90%) as a white solid. ¹H NMR (CDCl₃, 300 MHz) δ 7.68 (s, 1H), 7.52-7.47 (m, 6H), 4.59 (s, 2H), 1.36 (s, 9H).

d. Preparation of Compound

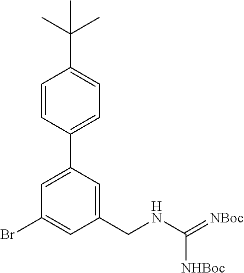

A 25-mL round bottom flask equipped with a magnetic stirrer, a condenser and a nitrogen in/outlet adapter was charged with 3-bromo-4′-(tert-butyl)-5-(chloromethyl)-1,1′-biphenyl 37c (600 mg, 1.77 mmol), DMF (5 mL), K₂CO₃ (329 mg, 2.39 mmol), and 1,3-bis(tert-butoxycarbonyl)guanidine (490 mg, 1.9 mmol) The reaction mixture was stirred at 50° C. for 3 hours. The reaction mixture was diluted with EtOAc (60 mL), washed with water (20 mL), 10% LiCl (20 mL), brine (20 mL), dried over Na₂SO₄, concentrated, and purified on silica gel. Elution with 5% EtOAc/hexanes afforded the title compound (700 mg, 70%) as a white solid. ¹H NMR (CDCl₃, 300 MHz) δ 9.51 (broad s, 1H), 9.36 (broad s, 1H), 7.60 (s, 1H), 7.51 (s, 1H), 7.47 (m, 4H), 7.38 (s, 1H), 5.18 (s, 2H), 1.50 (s, 9H), 1.39 (s, 9H), 1.36 (s, 9H).

### Example-38

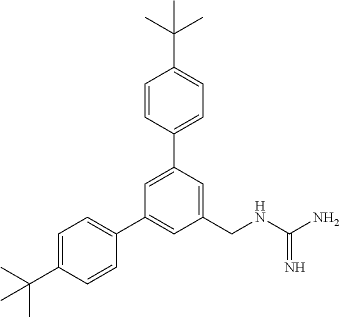 [SMILES: CC(C)(C)c1ccc(-c2cc(CNC(=N)N)cc(-c3ccc(C(C)(C)C)cc3)c2)cc1]

A 10-mL vial was added 38a (20 mg, 0.03 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (9.0 mg, 69%) as a white solid. ¹H NMR (CDCl₃, 300 MHz) δ 8.58 (broad s, 1H), 7.74 (s, 1H), 7.54 (d, 4H, J=8.40 Hz), 7.48 (d, 4H, J=8.10 Hz), 7.41 (s, 3H), 4.40 (d, 2H, J=5.1 Hz), 1.35 (s, 18H).

The requisite intermediates were prepared as follows:

a. Preparation of Compound

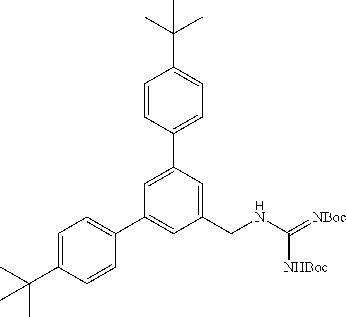

A 25-mL round bottom flask equipped with a magnetic stirrer, a condenser and a nitrogen in/outlet adapter was charged with di-tert-butyl ((((5-bromo-4′-(tert-butyl)-[1,1′-biphenyl]-3-yl)methyl)amino)methylene)dicarbamate 37d (50 mg, 0.09 mmol), (4-(tert-butyl)phenyl)boronic acid (27 mg, 0.13 mmol), water/dioxane (1 mL/3 ml), K₂CO₃ (25 mg, 0.18 mmol). The resulting solution was degassed for 5 minutes, then Pd(PPh₃)₄ (10 mg, 0.009 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated and purified on silica gel. Elution with 5% EtOAc/hexanes afforded the title compound (21 mg, 64%) as a white foam. ¹H NMR (CDCl₃, 300 MHz) δ 9.50 (broad s, 1H), 9.36 (broad s, 1H), 7.49-7.68 (m, 10H), 7.30 (s, 1H), 5.28 (s, 2H), 1.50 (s, 9H), 1.37 (s, 27H).

### Example-39

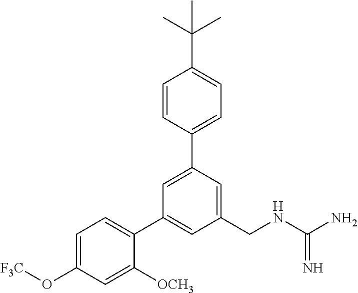 [SMILES: COc1cc(OC(F)(F)F)ccc1-c1cc(CNC(=N)N)cc(-c2ccc(C(C)(C)C)cc2)c1]

A 10-mL vial was added di-tert-butyl ((((4″-(tert-butyl)-2-methoxy-4-(trifluoromethoxy)-[1,1′:3′,1″-terphenyl]-5′-yl)methyl)amino)methylene)dicarbamate 39a (34 mg, 0.05 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (17 mg, 71%) as a white solid. ¹H NMR (CDCl₃, 300 MHz) δ 8.58 (broad s, 1H), 7.66 (s, 1H), 7.28-7.54 (m, 9H), 7.18 (s, 1H), 4.40 (d, 2H, J=5.1 Hz), 3.83 (s, 3H), 1.35 (s, 9H).

The requisite intermediates were prepared as follows:

a. Preparation of Compound

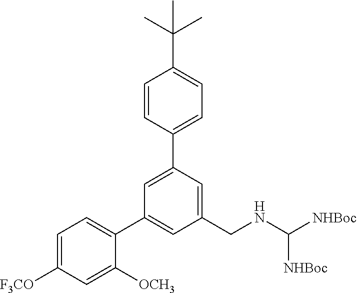 [SMILES: COc1cc(OC(F)(F)F)ccc1-c1cc(CNC(NC(=O)OC(C)(C)C)NC(=O)OC(C)(C)C)cc(-c2ccc(C(C)(C)C)cc2)c1]

A 25-mL round bottom flask equipped with a magnetic stirrer, a condenser and a nitrogen in/outlet adapter was charged with di-tert-butyl ((((5-bromo-4′-(tert-butyl)-[1,1′-biphenyl]-3-yl)methyl)amino)methylene)dicarbamate 37d (50 mg, 0.09 mmol), (2-methoxy-4-(trifluoromethoxy)phenyl)boronic acid (25 mg, 0.12 mmol), water/dioxane (1 mL/3 ml), K₂CO₃ (25 mg, 0.18 mmol). The resulting solution was degassed for 5 minutes, then Pd(PPh₃)₄ (10 mg, 0.009 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated and purified on silica gel. Elution with 5% EtOAc/hexanes afforded the title compound (38 mg, 63%) as a white foam. ¹H NMR (CDCl₃, 300 MHz) δ 9.51 (broad s, 1H), 9.36 (broad s, 1H), 7.27-7.58 (m, 9H), 7.19 (s, 1H), 5.28 (s, 2H), 3.85 (s, 3H), 1.48 (s, 9H), 1.38 (s, 18H).

### Example-40

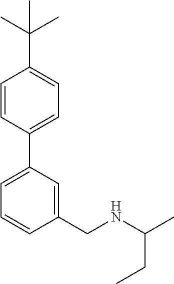 [SMILES: CCC(C)NCc1cccc(-c2ccc(C(C)(C)C)cc2)c1]

A 2-dram vial was added 4′-(tert-butyl)-3-(chloromethyl)-1,1′-biphenyl 2c (100 nag, 0.12 mmol), CH₃CN (1 mL), butan-2-amine (32 mg, 0.43 mmol). The sealed vial was stirred at room temperature for 12 hours. After cooling to room temperature, the solvent was removed and the residue was purified on silica gel. Elution with EtOAc afforded the title compound (80 mg, 70%) as a white solid. ¹H NMR (CDCl₃, 300 MHz) δ 8.42 (broad s, 1H), 7.41 (s, 4H), 6.82 (d, 1H, J=1.20 Hz), 6.80 (d, 1H, J=1.20 Hz), 4.24 (d, 2H, J=4.5 Hz), 3.86 (s, 3H), 3.58 (s, 3H), 1.33 (s, 9H).

### Example-41

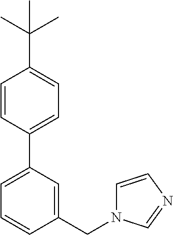 [SMILES: CC(C)(C)c1ccc(-c2cccc(Cn3ccnc3)c2)cc1]

A 2-dram vial was added 4′-(tert-butyl)-3-(chloromethyl)-1,1′-biphenyl 2c (30 mg, 0.12 mmol), CH₃CN (1 mL), 1H-imidazole (25 mg, 0.36 mmol). The sealed vial was heated to 80° C. for 2 hours. After cooling to room temperature, the solvent was removed and the residue was purified on silica gel. Elution with EtOAc afforded the title compound (27 mg, 79%) as colorless oil. ¹H NMR (CDCl₃, 300 MHz) δ 7.34-7.60 (m, 8H), 7.09-7.10 (m, 2H), 6.94 (s, 1H), 5.18 (s, 2H), 1.36 (s, 9H).

### Example-42

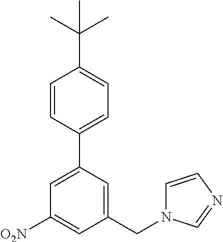 [SMILES: CC(C)(C)c1ccc(-c2cc(Cn3ccnc3)cc([N+](=O)[O-])c2)cc1]

A 2-dram vial was added 3-(bromomethyl)-4′-(tert-butyl)-5-nitro-1,1′-biphenyl 42b (150 mg, 0.43 mmol), CH₃CN (1 mL), 1H-imidazole (88 mg, 1.29 mmol). The sealed vial was heated to 80° C. for 2 hours. After cooling to room temperature, the solvent was removed and the residue was purified on silica gel. Elution with 10% EtOAc/hexanes afforded the title compound (83 mg, 58%) as a white solid. ¹H NMR (CDCl₃, 300 MHz) δ 8.39 (s, 1H), 7.98 (s, 1H), 7.63 (s, 1H), 7.61 (s, 1H), 7.50 (s, 4H), 7.11 (s, 1H), 6.96 (s, 1H), 5.29 (s, 2H), 1.35 (s, 9H).

The requisite intermediates were prepared as follows:

a. Preparation of Compound

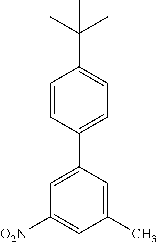 [SMILES: Cc1cc(-c2ccc(C(C)(C)C)cc2)cc([N+](=O)[O-])c1]

A 100-mL round bottom flask equipped with a magnetic stirrer, a condenser and a nitrogen in/outlet adapter was charged with 1-bromo-3-methyl-5-nitrobenzene (500 mg, 2.31 mmol), (4-(tert-butyl)phenyl)boronic acid (595 mg, 3.0 mmol), water/dioxane (6 mL/20 ml), K₂CO₃ (639 mg, 4.63 mmol). The resulting solution was degassed for 5 min, then Pd(PPh₃)₄ (80 mg, 0.07 mmol) was added. The reaction mixture was heated to 100° C. for 2 h. After cooling to room temperature, the reaction mixture was diluted with EtOAc and washed with saturated NaHCO₃, brine, dried over Na₂SO₄. The organic layer was concentrated and purified on silica gel. Elution with 5% EtOAc/hexanes afforded the title compound (605 mg, 97%) as light yellow oil. ¹H NMR (CDCl₃, 300 MHz) δ 8.24 (s, 1H), 7.99 (s, 1H), 7.70 (s, 1H), 7.48-7.56 (m, 4H), 2.51 (s, 3H), 1.36 (s, 9H).

b. Preparation of Compound

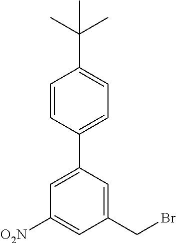 [SMILES: CC(C)(C)c1ccc(-c2cc(CBr)cc([N+](=O)[O-])c2)cc1]

A 100-mL round bottom flask equipped with a magnetic stirrer, a condenser and a nitrogen in/outlet adapter was charged with 42a (307 mg, 1.14 mmol), CCl₄ (15 mL), and NBS (213 mg, 1.20 mmol). The reaction mixture was refluxed for 4 hours by heating with bulb light. After cooling to room temperature, the reaction mixture was added hexanes (60 mL). The solid was removed by filtration and the filtrate was concentrated under reduced pressure and purified on silica gel. Elution with 5% EtOAc/hexanes afforded the title compound (238 mg, 60%) as off-white solid. ¹H NMR (CDCl₃, 300 MHz) δ 8.38 (s, 1H), 8.20 (s, 1H), 7.91 (s, 1H), 7.50-7.59 (m, 4H), 4.59 (s, 2H), 1.38 (s, 9H).

### Example-43

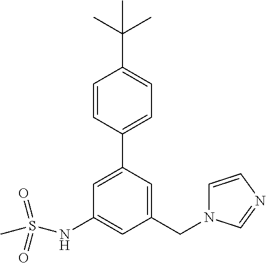 [SMILES: CC(C)(C)c1ccc(-c2cc(Cn3ccnc3)cc(NS(C)(=O)=O)c2)cc1]

A 15-mL round bottom flask equipped with a magnetic stirrer under nitrogen was charged with 5-((1H-imidazol-1-yl)methyl)-4′-(tert-butyl)-[1,1′-biphenyl]-3-amine 43a (31 mg, 0.1 mmol), CH₂Cl₂ (2 mL), and triethylamine (28 μL, 0.2 mmol). After cooling to 0° C., methanesulfonyl chloride (12 μL, 0.15 mmol) was added via a syringe. The resulting reaction mixture was stirred at room temperature overnight. The reaction mixture was diluted with CH₂Cl₂ and washed with saturated NaHCO₃, brine, dried over Na₂SO₄, concentrated under reduced pressure and purified on silica gel. Elution with 10% MeOH/EtOAc afforded the title compound (24 mg, 61%) as a white solid. ¹H NMR (CDCl₃, 300 MHz) δ 7.60 (s, 1H), 7.47 (s, 5H),), 7.16 (s, 1H), 7.11 (s, 1H), 6.96 (s, 1H), 6.90 (s, 1H), 5.18 (s, 2H), 3.03 (s, 3H), 1.36 (s, 9H).

The requisite intermediates were prepared as follows:

a. Preparation of Compound

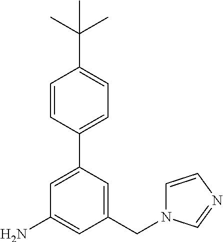 [SMILES: CC(C)(C)c1ccc(-c2cc(N)cc(Cn3ccnc3)c2)cc1]

A 50-mL round bottom flask equipped with a magnetic stirrer under N₂ was charged with 1-((4′-(tert-butyl)-5-nitro-[1,1′-biphenyl]-3-yl)methyl)-1H-imidazole 42 (90 mg, 0.27 mmol), MeOH (10 ml). The reaction mixture was degassed for 5 min, then Pd/C (10%, 20 mg) was added. The reaction mixture was stirred for 12 h under H₂ balloon. The catalyst was removed by passing through Celite plug. The filtrate was concentrated to afford the title compound (80 mg, 97%) as white solid. ¹H NMR (CDCl₃, 300 MHz) □ 7.93 (s, 1H), 7.60 (s, 1H), 7.43 (s, 4H), 7.15 (s, 1H), 7.09 (s, 1H), 6.94 (s, 1H), 6.83 (s, 1H), 6.76 (s, 1H), 6.36 (s, 1H), 5.07 (s, 2H), 1.34 (s, 9H).

### Example-44

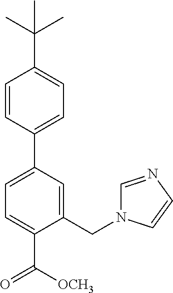 [SMILES: COC(=O)c1ccc(-c2ccc(C(C)(C)C)cc2)cc1Cn1ccnc1]

A 10-ml vial was added methyl 3-(bromomethyl)-4′-(tert-butyl)-[1,1′-biphenyl]-4-carboxylate 44b (250 mg, ˜0.62 mmol), CH₃CN (2 mL), 1H-imidazole (126 mg, 1.86 mmol). The sealed vial was heated to 80° C. for 2 hours. After cooling to room temperature, the solvent was removed and the residue was purified on silica gel. Elution with EtOAc afforded the title compound (200 mg) as a white solid. ¹H NMR (CDCl₃, 300 MHz) δ 8.10 (d, 2H, J=8.10 Hz), 7.61 (s, 1H), 7.59 (d, 2H, J=8.10 Hz), 7.44 (s, 4H), 7.12 (s, 1H), 7.06 (s, 1H), 6.97 (s, 1H), 5.64 (s, 2H), 3.93 (s, 3H), 1.34 (s, 9H).

The requisite intermediates were prepared as follows:

a. Preparation of Compound

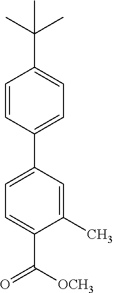 [SMILES: COC(=O)c1ccc(-c2ccc(C(C)(C)C)cc2)cc1C]

A 200-mL round bottom flask equipped with a magnetic stirrer, a condenser and a nitrogen in/outlet adapter was charged with methyl 4-bromo-2-methylbenzoate (1.50 g, 6.55 mmol), (4-(tert-butyl)phenyl)boronic acid (1.69 g, 8.5 mmol), water/dioxane (10 mL/30 mL), K₂CO₃ (1.81 g, 13.1 mmol). The resulting solution was degassed for 5 minutes, then Pd(PPh₃)₄ (150 mg, 0.13 mmol) was added. The reaction mixture was heated to 100° C. for 2 hours. After cooling to room temperature, the reaction mixture was diluted with EtOAc and washed with saturated NaHCO₃, brine, dried over Na₂SO₄. The organic layer was concentrated and purified on silica gel. Elution with 10% EtOAc/hexanes afforded the title compound (1.80 g, 97%) as colorless oil. ¹H NMR (CDCl₃, 300 MHz) δ 7.98 (d, 1H, J=8.10 Hz), 7.60-7.43 (m, 7H), 3.91 (s, 3H), 2.67 (s, 3H), 1.36 (s, 9H).

b. Preparation of Compound

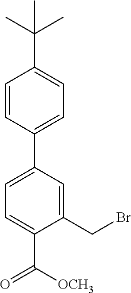 [SMILES: COC(=O)c1ccc(-c2ccc(C(C)(C)C)cc2)cc1CBr]

A 100-mL round bottom flask equipped with a magnetic stirrer, a condenser and a nitrogen in/outlet adapter was charged with methyl 4′-(tert-butyl)-3-methyl-[1,1′-biphenyl]-4-carboxylate 44a (1.07 g, 6.02 mmol), CCl₄ (15 mL), and NBS (742 mg, 6.62 mmol). The reaction mixture was refluxed for 5 hours by heating with bulb light. The starting material was consumed by TLC. After cooling to room temperature, the reaction mixture was added hexanes (60 mL). The solid was removed by filtration and the filtrate was concentrated under reduced pressure and purified on silica gel. Elution with 5% EtOAc/hexanes afforded the title compound (1.5 g, 100%) as a yellow oil. The bromide was used in next step without further identification and purification.

### Example-45

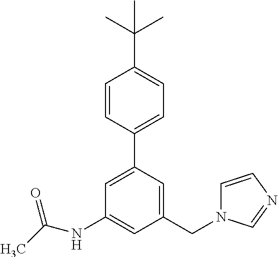 [SMILES: CC(=O)Nc1cc(Cn2ccnc2)cc(-c2ccc(C(C)(C)C)cc2)c1]

A 15-mL round bottom flask equipped with a magnetic stirrer under nitrogen was charged with 5-((1H-imidazol-1-yl)methyl)-4′-(tert-butyl)-[1,1′-biphenyl]-3-amine 43a (31 mg, 0.1 mmol), CH₂Cl₂ (2 mL), and triethylamine (28 μL, 0.2 mmol). After cooling to 0° C., acetic anhydride (15 μL, 0.15 mmol) was added via a syringe. The resulting reaction mixture was stirred at room temperature overnight. The reaction mixture was diluted with CH₂Cl₂ and washed with saturated NaHCO₃, brine, dried over Na₂SO₄, concentrated and purified using silica gel. Elution with 10% MeOH/EtOAc afforded the title compound (25 mg, 75%) as a white solid. ¹H NMR (CDCl₃, 300 MHz) δ 8.13 (s, 1H), 7.74 (s, 1H), 7.55 (s, 1H), 7.40-7.48 (m, 4H), 7.08 (s, 2H), 6.94 (s, 1H), 5.14 (s, 2H), 2.17 (s, 3H), 1.34 (s, 9H).

### Example-46

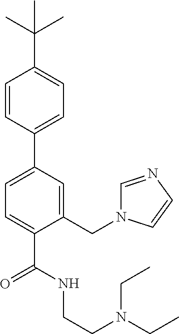 [SMILES: CCN(CC)CCNC(=O)c1ccc(-c2ccc(C(C)(C)C)cc2)cc1Cn1ccnc1]

A 10-mL round bottom flask equipped with a magnetic stirrer under nitrogen was charged with 3-((1H-imidazol-1-yl)methyl)-4′-(tert-butyl)-[1,1′-biphenyl]-4-carboxylic acid 46a (33 mg, 0.10 mmol), CH₂Cl₂ (1 mL). DMF (1 drop), oxalyl chloride (4 drops) was added. The reaction mixture was stirred at room temperature for 1 hour. The solvent was removed and the residue was dissolved in CH₂Cl₂ (2 mL), then N¹,N¹-diethylethane-1,2-diamine (4 drops) was added. The resulting reaction mixture was stirred at room temperature for 1 hour. The reaction mixture was concentrated and purified on silica gel. Elution with CH₂Cl₂ to (10/89/1: MeOH/CHCl₃/ammonium hydroxide) afforded the title compound (19 mg, 46%) as a white solid. ¹H NMR (CDCl₃, 300 MHz) δ 7.57 (s, 1H), 7.47-7.54 (m, 2H), 7.44 (s, 5H), 7.03 (s, 1H), 6.93 (s, 2H), 6.67 (broad s, 1H), 5.46 (s, 2H), 3.44 (q, 2H, J=6.3 Hz), 2.62 (t, 2H, J=6.0 Hz), 1.95 (s, 3H), 2.55 (q, 4H, J=7.2 Hz), 1.34 (s, 9H), 1.02 (t, 6H, J=7.2 Hz).

The requisite intermediate was prepared as follows:

a. Preparation of Compound

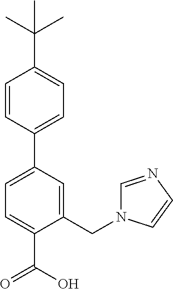 [SMILES: CC(C)(C)c1ccc(-c2ccc(C(=O)O)c(Cn3ccnc3)c2)cc1]

A 25-mL round bottom flask equipped with a magnetic stirrer, a condenser and a nitrogen in/outlet adapter was charged with methyl 3-((1H-imidazol-1-yl)methyl)-4′-(tert-butyl)-[1,1′-biphenyl]-4-carboxylate 44 (60 mg, 0.17 mmol), LiOH (40 mg, 1.7 mmol), THF/H₂O (2.0 ml/1.0 mL). The reaction mixture was heated to 80° C. for 12 hours. After cooling to room temperature, the solvent was removed and the residue was added 4 N HCl (5 mL). The solid was collected by filtration and washed with H₂O. After drying, there was obtained the title compound (50 mg, 86%) as a white solid. The acid was used in next step without further identification.

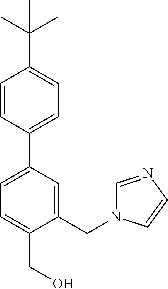 [SMILES: CC(C)(C)c1ccc(-c2ccc(CO)c(Cn3ccnc3)c2)cc1]

### Example-47

A 25-mL round bottom flask equipped with a magnetic stirrer, and a condenser under nitrogen was charged with methyl 3-((1H-imidazol-1-yl)methyl)-4′-(tert-butyl)-[1,1′-biphenyl]-4-carboxylate 44 (55 mg, 0.16 mmol), THF (3 mL) After cooling to 0° C., LiBH₄ (7 mg, 0.32 mmol) was added in one portion. The resulting reaction mixture was warmed to room temperature then heated to 85° C. for 3 hours. After being allowed to cool to room temperature, acetone (1 ml) was added to the reaction mixture and stirred for 10 min. The solvent was removed and the residue was diluted with ethyl acetate. Washing with 1 N HCl, followed by saturated NaHCO₃, brine, the organic layers were dried over Na₂SO₄, concentrated and purified with silica gel. Elution with 10% MeOH/EtOAc afforded the title compound (42 mg, 84%) as a white solid. ¹H NMR (CDCl₃, 300 MHz) δ 7.52-7.54 (m, 2H), 7.44 (s, 4H), 7.44 (s, 4H), 7.42 (s, 1H), 7.23 (s, 1H), 7.04 (s, 1H), 6.91 (s, 2H), 5.32 (s, 3H), 4.68 (s, 3H), 1.34 (s, 9H).

### Example-48

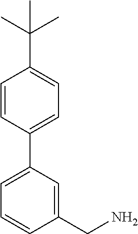 [SMILES: CC(C)(C)c1ccc(-c2cccc(CN)c2)cc1]

A 100-mL round bottom flask equipped with a magnetic stirrer, a condenser and a nitrogen in/outlet adapter was charged with 4′-(tert-butyl)-[1,1′-biphenyl]-3-carbonitrile 48a (103 mg, 0.44 mmol), THF/toluene (5 mL/5 mL). After being allowed to cool to 0° C., LiAlH₄ (84 mg, 2.2 mmol) was added to the reaction mixture. The reaction mixture was heated to 100° C. and stirred for 3 hours. After cooling to 0° C., the reaction mixture was diluted with EtOAc (40 mL) and quenched with 15% NaOH (0.44 mL), water (2.0 mL). The organic layer was decanted, dried over Na₂SO₄, concentrated and purified on silica gel. Elution with 5% MeOH/CH₂Cl₂ afforded the title compound (41 mg, 39% in yield) as colorless oil. ¹H NMR (CDCl₃, 300 MHz) δ 7.36-7.56 (m, 7H), 7.24-7.29 (m, 1H), 3.93 (s, 2H), 1.35 (s, 9H).

The requisite intermediate was prepared as follows:

a. Preparation of Compound

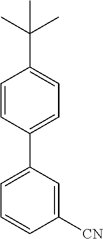 [SMILES: CC(C)(C)c1ccc(-c2cccc(C#N)c2)cc1]

A 100-mL round bottom flask equipped with a magnetic stirrer, a condenser and a nitrogen in/outlet adapter was charged with 3-bromobenzonitrile (500 mg, 2.75 mmol), (4-(tert-butyl)phenyl)boronic acid (636 mg, 3.57 mmol), water/dioxane (6 mL/20 ml), K₂CO₃ (759 mg, 5.5 mmol). The resulting solution was degassed for 5 min, then Pd(PPh₃)₄ (92 mg, 0.08 mmol) was added. The reaction mixture was heated to 100° C. for 2 h. After cooling to room temperature, the reaction mixture was diluted with EtOAc and washed with saturated NaHCO₃, brine, dried over Na₂SO₄. The organic layer was concentrated and purified on silica gel. Elution with 5% hexanes afforded the title compound (588 mg, 91%) white solid. ¹H NMR (CDCl₃, 300 MHz) δ 7.55 (d, 2H, J=8.4 Hz), 7.45 (d, 2H, J=8.4 Hz), 7.32-7.37 (m, 3H), 7.17-7.19 (m, 1H), 2.42 (s, 3H).

### Example-49

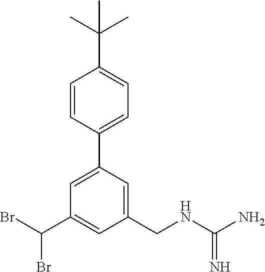 [SMILES: CC(C)(C)c1ccc(-c2cc(CNC(=N)N)cc(C(Br)Br)c2)cc1]

A 2-dram vial was added the mixture from 50c (32 mg, 0.05 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (8.6 mg, 36%) as a white foam. ¹H NMR (CDCl₃, 300 MHz) δ 8.64 (broad s, 1H), 7.72 (s, 1H), 7.49 (s, 4H), 7.42 (s, 1H), 7.40 (s, 1H), 6.65 (s, 1H), 4.38 (d, 2H, J=5.4 Hz), 1.34 (s, 9H).

### Example-50

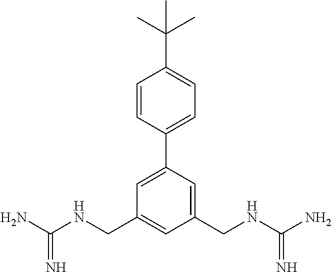 [SMILES: CC(C)(C)c1ccc(-c2cc(CNC(=N)N)cc(CNC(=N)N)c2)cc1]

A 2-dram vial was added tetra-tert-butyl ((((4′-(tert-butyl)-[1,1′-biphenyl]-3,5-diyl)bis(methylene))bis(azanediyl))bis(methanetriyl))tetracarbamate (30 mg, 0.04 mmol), CH₂Cl₂ (1 mL), and TFA (1 mL). The sealed vial was stirred at 50° C. for 1 hour. The solvent was removed and the residue was dissolved in CH₂Cl₂ (30 mL) and washed with 4 N NaOH (5 mL), brine (5 mL), dried over Na₂SO₄, concentrated to afford the title compounds (10 mg, 71%) as an off-white foam. ¹H NMR (CD₃OD, 300 MHz) δ 7.33-7.53 (m, 6H), 7.19 (s, 1H), 4.38 (s, 4H), 1.27 (s, 9H).

The requisite intermediates were prepared as follows:

a. Preparation of Compound

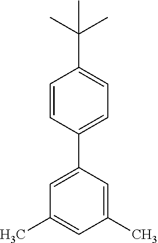

A 100-mL round bottom flask equipped with a magnetic stirrer, a condenser and a nitrogen in/outlet adapter was charged with 1-bromo-4-(tert-butyl)benzene (450 mg, 2.11 mmol), (3,5-dimethylphenyl)boronic acid (475 mg, 3.17 mmol), water/dioxane (6 mL/20 mL), K₂CO₃ (582 mg, 4.22 mmol). The resulting solution was degassed for 5 minutes, then Pd(PPh₃)₄ (80 mg, 0.07 mmol) was added. The reaction mixture was heated to 100° C. for 2 hours. After cooling to room temperature, the reaction mixture was diluted with EtOAc and washed with saturated NaHCO₃, brine, dried over Na₂SO₄. The organic layer was concentrated and purified on silica gel. Elution with hexanes afforded the title compound (336 mg, 67%) as colorless oil. ¹H NMR (CDCl₃, 300 MHz) δ 7.51 (d, 2H, J=8.10 Hz), 7.43 (d, 2H, J=8.70 Hz), 7.19 (s, 2H), 6.97 (s, 1H), 2.37 (s, 6H), 1.35 (s, 9H).

b. Preparation of Compound

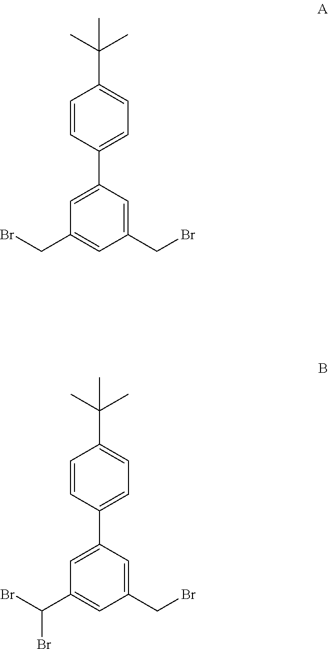 [SMILES: CC(C)(C)c1ccc(-c2cc(CBr)cc(CBr)c2)cc1 ; CC(C)(C)c1ccc(-c2cc(CBr)cc(C(Br)Br)c2)cc1]

A 100-mL round bottom flask equipped with a magnetic stirrer, a condenser and a nitrogen in/outlet adapter was charged with 4′-(tert-butyl)-3,5-dimethyl-1,1′-biphenyl 50a (336 mg, 1.41 mmol), CCl₄ (10 mL), AIBN (23 mg, 0.14 mmol), and NBS (627 mg, 3.53 mmol). The reaction mixture was heated to 85° C. for 2 hours. The starting material was consumed by checking with TLC, developing with hexanes. After cooling to room temperature, the reaction mixture was added hexanes (60 mL). The solid was removed by filtration and the filtrate was concentrated to afford a crude mixture of A and B (wt=561 mg). The ratio of A to B was about 40 to 60 based on the NMR integral with chemical shift at 6.67 and 4.52. The crude mixture as used in next step without further purification.

c. Preparation of Compound

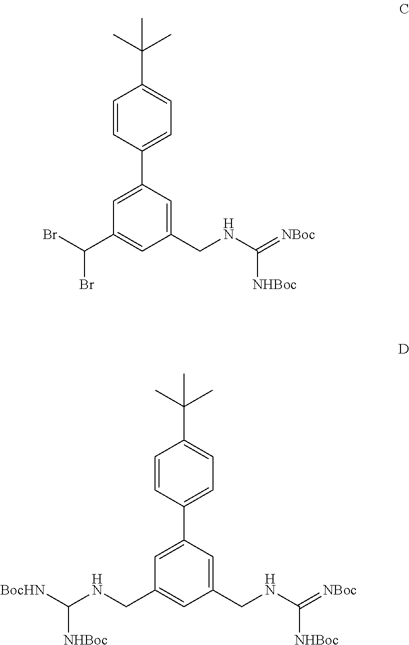

A 25-mL round bottom flask equipped with a magnetic stirrer, a condenser and a nitrogen in/outlet adapter was charged with the mixture of A and B, from 50b, (90 mg), DMF (2 mL), K₂CO₃ (94 mg, 0.68 mmol), and 1,3-bis(tert-butoxycarbonyl)guanidine (176 mg, 0.07 mmol) The reaction mixture was stirred at 50° C. for 12 hours. The reaction mixture was diluted with EtOAc (60 mL), washed with water (20 mL), 10% LiCl (20 mL), brine (20 mL), dried over Na₂SO₄, concentrated, and purified on silica gel. Elution with 5% EtOAc/hexanes afforded the title compound C (36 mg) and D (intermediate to Example 50) (30 mg) as a white solid. ¹H NMR of C (CDCl₃, 300 MHz) δ 9.47 (broad s, 1H), 9.32 (broad s, 1H), 7.41-7.61 (m, 7H), 6.67 (s, 1H), 5.22 (s, 2H), 1.49 (s, 9H), 1.40 (s, 9H), 1.35 (s, 9H); ¹H NMR of D (CDCl₃, 300 MHz) 9.47 (broad s, 1H), 9.32 (broad s, 1H), 7.41-7.51 (m, 6H), 7.01 (s, 1H), 5.19 (s, 2H), 1.47 (s, 18H), 1.32-1.35 (s, 27H).

### Examples-51 and 52

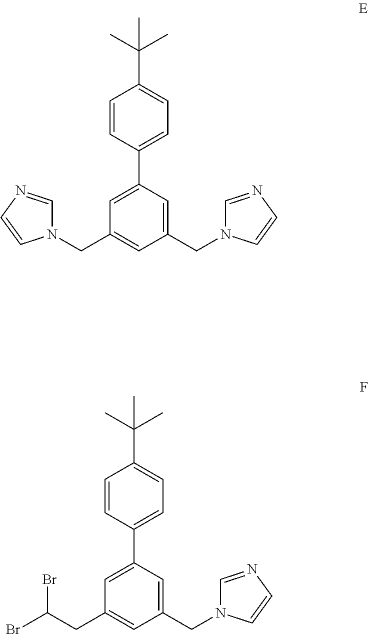 [SMILES: CC(C)(C)c1ccc(-c2cc(Cn3ccnc3)cc(Cn3ccnc3)c2)cc1 ; CC(C)(C)c1ccc(-c2cc(CC(Br)Br)cc(Cn3ccnc3)c2)cc1]

A 25-mL round bottom flask equipped with a magnetic stirrer, a condenser and a nitrogen in/outlet adapter was charged with the mixture of A and B from 50b (100 mg), DMF (2 mL), K₂CO₃ (70 mg, 0.51 mmol), and 1H-imidazole (40 mg, 0.50 mmol) The reaction mixture was stirred at 50° C. for 12 hours. The reaction mixture was diluted with EtOAc (60 mL), washed with water (20 mL), 10% LiCl (20 mL), brine (20 mL), dried over Na₂SO₄, concentrated, and purified on silica gel. Elution with CH₂Cl₂ to (10/89/1: MeOH/CHCl₃/ammonium hydroxide) afforded the title compounds E, Example 51, (22 mg) and F, Example 52, (15 mg) as a white solid. ¹H NMR of E (CDCl₃, 300 MHz) δ 7.56 (s, 2H), 7.38-7.45 (m, 4H), 7.27 (s, 2H), 7.10 (s, 2H), 6.91 (s, 2H), 6.86 (s, 1H), 5.14 (s, 4H), 1.34 (s, 9H); ¹H NMR of F (CDCl₃, 300 MHz) δ 7.71 (s, 1H), 7.59 (s, 1H), 7.38-7.46 (m, 4H), 7.25 (d, 1H, J=12 Hz), 7.12 (s, 1H), 6.94 (s, 1H), 6.64 (s, 1H), 5.19 (s, 2H), 1.34 (s, 9H).

### Example-53

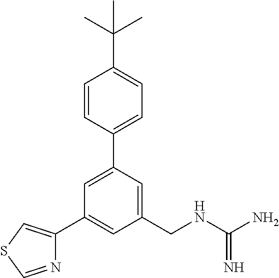 [SMILES: CC(C)(C)c1ccc(-c2cc(CNC(=N)N)cc(-c3cscn3)c2)cc1]

A 10-mL vial was added 53a (20 mg, 0.035 mmol), CH₂Cl₂ (1 mL), and TFA (1 mL) The sealed vial was stirred at 50° C. for 1 h. The solvent was removed and the residue was purified on silica gel. Elution with CH₂Cl₂ to (10/89/1: MeOH/CHCl₃/ammonium hydroxide) afforded the title compound (8.5 mg, 65%) as a white solid. ¹H NMR (CDCl₃, 300 MHz) □ 8.80 (s, 1H), 8.58 (broad s, 1H), 7.92 (s, 1H), 7.68 (s, 1H), 7.52 (s, 1H), 7.42-7.49 (m, 4H), 7.34 (s, 1H), 4.34 (d, 2H, J=4.5 Hz), 1.33 (s, 9H).

The requisite intermediates were prepared as follows:

a. Preparation of Compound

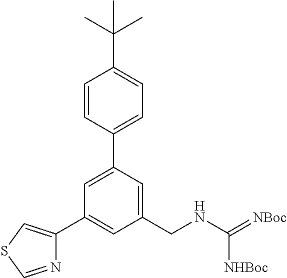

A 25-mL round bottom flask equipped with a magnetic stirrer, a condenser and a nitrogen in/outlet adapter was charged with di-tert-butyl ((((4′-(tert-butyl)-5-(4,4,5,5-tetramethyl-1,3,2-dioxaborolan-2-yl)-[1,1′-biphenyl]-3-yl)methyl)amino)methylene)dicarbamate 53b (30 mg, 0.05 mmol), 4-bromothiazole (11 mg, 0.07 mmol), water/dioxane (1 mL/3 ml), K₂CO₃ (15 mg, 0.11 mmol). The resulting solution was degassed for 5 minutes, then Pd(PPh₃)₄ (8 mg, 0.007 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated under reduced pressure and purified on silica gel. Elution with 10% EtOAc/hexanes afforded the title compound (25 mg, 68%) as a white solid. ¹H NMR (CDCl₃, 300 MHz) δ 9.47 (broad s, 1H), 9.32 (broad s, 1H), 8.88-8.9 (s, 1H), 8.06 (s, 1H), 7.84 (s, 1H), 7.55-7.60 (m, 411), 7.47 (d, 2H, J=8.10 Hz), 5.28 (s, 2H), 1.49 (s, 9H), 1.36 (s, 18H).

b. Preparation of Compound

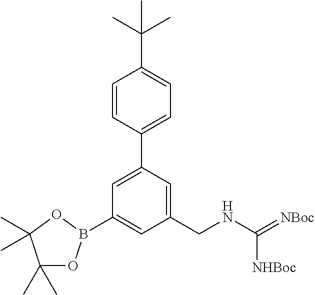

A 50-mL round bottom flask equipped with a magnetic stirrer, a condenser and a nitrogen in/outlet adapter was charged with di-tert-butyl ((((5-bromo-4′-(tert-butyl)-[1,1′-biphenyl]-3-yl)methyl)amino)methylene)dicarbamate 37d (618 mg, 1.1 mmol), 4,4,4′,4′,5,5,5′,5′-octamethyl-2,2′-bi(1,3,2-dioxaborolane) (362 mg, 1.42 mmol), dioxane (10 ml), KOAc (323 mg, 3.3 mmol). The resulting solution was degassed for 5 minutes, then Pd(dppf)Cl₂ (45 mg, 0.06 mmol) was added and the solution was carefully degassed. The reaction mixture was warmed to 80° C. and stirred for 5 hours. After cooling to room temperature, the reaction mixture was diluted with EtOAc and washed with saturated NaHCO₃, then brine and dried over Na₂SO₄. The organic layer was concentrated and purified on silica gel. Elution with 5% EtOAc/hexanes afforded the title compound (230 mg, 34%) as a white solid. ¹H NMR (CDCl₃, 300 MHz) δ 9.43 (broad s, 1H), 9.30 (broad s, 1H), 7.91 (s, 1H), 7.75 (s, 1H), 7.71 (s, 1H), 7.56 (d, 2H, J=8.40 Hz), 7.43 (d, 2H, J=8.40 Hz), 5.21 (s, 2H), 1.50 (s, 9H), 1.39 (s, 9H), 1.33-1.35 (m, 21H).

### Example-54

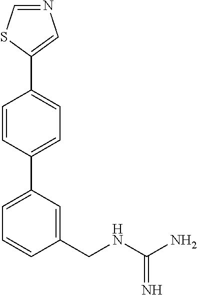 [SMILES: N=C(N)NCc1cccc(-c2ccc(-c3cncs3)cc2)c1]

A 10-mL vial was added 54a (25 mg, 0.05 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (10 mg, 66%) as a white solid. ¹H NMR (CD₃OD, 300 MHz) δ 9.01 (s, 1H), 8.25 (s, 1H), 7.73-7.82 (m, 4H), 7.65-7.70 (m, 2H), 7.53 (t, 1H, J=7.50 Hz), 7.38 (d, 1H, J=6.10 Hz), 4.52 (s, 2H).

The requisite intermediate was prepared as follows:

a. Preparation of Compound

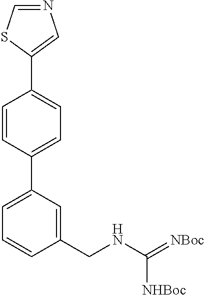

A 25-mL round bottom flask equipped with a magnetic stirrer, a condenser and a nitrogen in/outlet adapter was charged with 55a (40 mg, 0.07 mmol), 5-bromothiazole (14 mg, 0.09 mmol), water/dioxane (1 mL/3 ml), K₂CO₃ (20 mg, 0.14 mmol). The resulting solution was degassed for 5 minutes, then Pd(PPh₃)₄ (10 mg, 0.009 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated under reduced pressure and purified on silica gel. Elution with 10% EtOAc/hexanes afforded the title compound (25 mg, 68%) as a white solid. ¹H NMR (CDCl₃, 300 MHz) δ 9.47 (broad s, 1H), 9.32 (broad s, 1H), 8.78 (s, 1H), 8.11 (s, 1H), 7.56-7.66 (m, 5H), 7.50 (d, 1H, J=7.80 Hz), 7.39 (t, 1H, J=7.50 Hz), 7.25 (d, 1H, J=7.50 Hz), 5.21 (s, 2H), 1.50 (s, 9H), 1.35 (s, 9H).

### Example-55

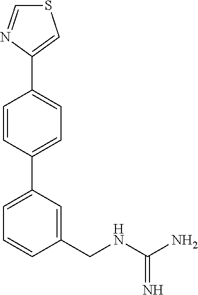 [SMILES: N=C(N)NCc1cccc(-c2ccc(-c3cscn3)cc2)c1]

A 10-mL vial was added 55b (20 mg, 0.04 mmol), CH₂Cl₂ (1 mL), and TFA (1 mL). The sealed vial was stirred at 50° C. for 1 hour. The solvent was removed and the residue was purified on silica gel. Elution with CH₂Cl₂ to (10/89/1: MeOH/CHCl₃/ammonium hydroxide) afforded the title compound (71 mg, 59%) as a white solid. ¹H NMR (CD₃OD, 300 MHz) δ 9.12 (d, 1H, J=2.1 Hz), 8.07 (d, 2H, J=8.10 Hz), 7.97 (d, 1H, J=1.8 Hz), 7.75 (d, 2H, J=8.10 Hz), 7.67-7.70 (m, 2H), 7.53 (t, 1H, J=8.40 Hz), 7.37 (d, 1H, J=7.50 Hz), 4.52 (s, 2H).

The required intermediate was prepared as follows:

a. Preparation of Compound

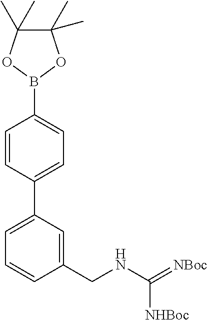

A 50-mL round bottom flask equipped with a magnetic stirrer, a condenser and a nitrogen in/outlet adapter was charged with 30b (540 mg, 1.1 mmol), 4,4,4′,4′,5,5,5′,5′-octamethyl-2,2′-bi(1,3,2-dioxaborolane) (362 mg, 1.42 mmol), dioxane (10 ml), KOAc (323 mg, 3.3 mmol). The resulting solution was degassed for 5 minutes, then Pd(dppf)Cl₂ (45 mg, 0.06 mmol) was added and the solution was carefully degassed. The reaction mixture was warmed to 80° C. and stirred for 12 hours. After cooling to room temperature, the reaction mixture was diluted with EtOAc and washed with saturated NaHCO₃, brine, dried over Na₂SO₄. The organic layer was concentrated and purified on silica gel. Elution with 5% EtOAc/hexanes afforded the title compound (352 mg, 59%) as a white solid. ¹H NMR (CDCl₃, 300 MHz) δ 9.43 (broad s, 1H), 9.30 (broad s, 1H), 7.86 (d, 2H, J=7.50 Hz), 7.58 (d, 2H, J=7.80 Hz), 7.55 (s, 1H), 7.48 (d, 1H, J=7.80 Hz), 7.36 (t, 1H, J=7.80 Hz), 7.24 (m, 1H), 5.21 (s, 2H), 1.49 (s, 9H), 1.35 (s, 9H), 1.25 (s, 12H).

b. Preparation of Compound

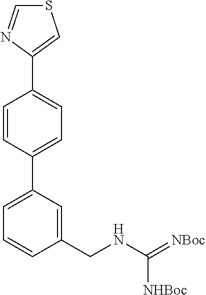

A 25-mL round bottom flask equipped with a magnetic stirrer, a condenser and a nitrogen in/outlet adapter was charged with 55a (40 mg, 0.07 mmol), 4-bromothiazole (14 mg, 0.09 mmol), water/dioxane (1 mL/3 ml), K₂CO₃ (20 mg, 0.14 mmol). The resulting solution was degassed for 5 minutes, then Pd(PPh₃)₄ (10 mg, 0.009 mmol) was added. The reaction mixture was heated to 100° C. for 2 hours. After being allowed to cool to room temperature, the reaction mixture was diluted with EtOAc (30 mL) and washed with saturated NaHCO₃ (10 mL), brine (10 mL), dried over Na₂SO₄. The organic layer was concentrated under reduced pressure and purified on silica gel. Elution with 10% EtOAc/hexanes afforded the title compound (28 mg, 76%) as a white solid. ¹H NMR (CDCl₃, 300 MHz) δ 9.49 (broad s, 1H), 9.35 (broad s, 1H), 8.90-8.91 (m, 1H), 8.00 (d, 1H, J=7.80 Hz), 7.67 (d, 1H, J=7.50 Hz), 7.35 (s, 1H), 7.58-7.59 (m, 1H), 7.53 (d, 1H, J=7.80 Hz), 7.39 (t, 1H, J=7.80 Hz), 7.24-7.26 (m, 1H), 3.25 (s, 2H), 1.50 (s, 9H), 1.36 (s, 9H).

### Example-56

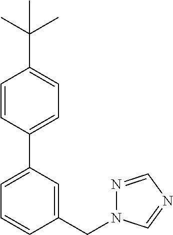 [SMILES: CC(C)(C)c1ccc(-c2cccc(Cn3cncn3)c2)cc1]

A 25-mL round bottom flask equipped with a magnetic stirrer, a condenser and a nitrogen in/outlet adapter was charged with 4′-(tert-butyl)-3-(chloromethyl)-1,1′-biphenyl 2c (82 mg, 0.125 mmol), DMF (1 mL), K₂CO₃ (35 mg, 0.25 mmol), and 1H-1,2,4-triazole (88 mg, 1.28 mmol) The reaction mixture was stirred at 80° C. for 12 hours. After cooling to room temperature, the solvent was removed and the residue was purified on silica gel. Elution with 50% EtOAc/hexanes afforded the title compound (25 mg, 27%) as a white solid. ¹H NMR (CDCl₃, 300 MHz) δ 8.09 (s, 1H), 7.98 (s, 1H), 7.56 (d, 1H, J=7.2 Hz), 7.40-7.50 (m, 6H), 7.21 (d, 1H, J=7.8 Hz), 5.40 (s, 2H), 1.35 (s, 9H).

### Example 57

The following can illustrate representative pharmaceutical dosage forms, containing a compound of formula I (‘Compound X’) or a pharmaceutically acceptable salt thereof, for therapeutic or prophylactic use in humans.

| (i) Tablet 1 | mg/tablet |
|---|---|
| Compound X= | 100.0 |
| Lactose | 77.5 |
| Povidone | 15.0 |
| Croscarmellose sodium | 12.0 |
| Microcrystalline cellulose | 92.5 |
| Magnesium stearate | 3.0 |
| | 300.0 |

| (ii) Tablet 2 | mg/tablet |
|---|---|
| Compound X= | 20.0 |
| Microcrystalline cellulose | 410.0 |
| Starch | 50.0 |
| Sodium starch glycolate | 15.0 |
| Magnesium stearate | 5.0 |
| | 500.0 |

| (iii) Capsule | mg/capsule |
|---|---|
| Compound X= | 10.0 |
| Colloidal silicon dioxide | 1.5 |
| Lactose | 465.5 |
| Pregelatinized starch | 120.0 |
| Magnesium stearate | 3.0 |
| | 600.0 |

| (iv) Injection 1 (1 mg/ml) | mg/ml |
|---|---|
| Compound X= (free acid form) | 1.0 |
| Dibasic sodium phosphate | 12.0 |
| Monobasic sodium phosphate | 0.7 |
| Sodium chloride | 4.5 |
| 1.0 N Sodium hydroxide solution | q.s. |
| (pH adjustment to 7.0-7.5) | |
| Water for injection | q.s. ad 1 mL |

| (v) Injection 2 (10 mg/ml) | mg/ml |
|---|---|
| Compound X= (free acid form) | 10.0 |
| Monobasic sodium phosphate | 0.3 |
| Dibasic sodium phosphate | 1.1 |
| Polyethylene glycol 400 | 200.0 |
| 1.0 N Sodium hydroxide solution | q.s. |
| (pH adjustment to 7.0-7.5) | |
| Water for injection | q.s. ad 1 mL |

| (vi) Aerosol | mg/can |
|---|---|
| Compound X= | 20.0 |
| Oleic acid | 10.0 |
| Trichloromonofluoromethane | 5,000.0 |
| Dichlorodifluoromethane | 10,000.0 |
| Dichlorotetrafluoroethane | 5,000.0 |

The above formulations may be obtained by conventional procedures well known in the pharmaceutical art.

All publications, patents, and patent documents are incorporated by reference herein, as though individually incorporated by reference. The invention has been described with reference to various specific and preferred embodiments and techniques. However, it should be understood that many variations and modifications may be made while remaining within the spirit and scope of the invention.

1. A method for treating a bacterial infection in a mammal comprising administering to the mammal an effective amount of a compound of formula I: 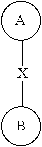
A is a ring selected from phenyl and thiazolyl, which ring is substituted with one or more R¹ and which ring is optionally substituted with one or more R^{b};
B is a ring selected from phenyl, pyridyl, benzothiazole, and thiazolyl, which ring is substituted with one or more R^{a} and which ring is optionally substituted with one or more R^{b};
R¹ is halo, —C(═O)NR^{v}R^{w}, phenyl, (C₁-C₆)alkyl, thiazolyl, R^{m}, or (C₃-C₆)cycloalkyl, which phenyl, (C₁-C₆)alkyl, thiazolyl, or (C₃-C₆)cycloalkyl is optionally substituted with one or more phenyl, thiazolyl or —C(═O)NR^{v}R^{w};
each R^{a} is independently:
a) —N⁺(R^{ac})₃Z⁻,
b) —NR^{d}R^{e},
c) —C(═NR^{c})—NR^{d}R^{e},
d) —NR^{f}—C(═NR^{c})—NR^{d}R^{e},
e) —NR^{f}—C(═NR^{c})—R^{c},
f) —NR^{f}—NR^{f}—C(═NR^{c})—NR^{d}R^{e},
g) —C(H)═N—NR^{f}—C(═NR^{c})—NR^{d}R^{e},
h) —C(═O)—NR^{f}—C(═NR^{c})—NR^{d}R^{e},
i) —C(═O)—NR^{f}—C(═NR^{c})—R^{c},
j) —(C₁-C₆)alkyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, and R^{j},
k) —(C₂-C₆)alkoxy that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, and R^{j}, or
l) phenyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e}, —NR^{f}—C(═NR^{c})—R^{c}, —NR^{f}—NR^{f}—C(═NR^{c})—NR^{d}R^{e}, —C(H)═N—NR^{f}—C(═NR^{c})—NR^{d}R^{e}, —C(═O)—NR^{f}—C(═NR^{c})—NR^{d}R^{e}, —C(═O)—NR^{f}—C(═NR^{c})—R^{c}, R^{j}, and R^{k}
each R^{b} is independently H, (C₁-C₆)alkyl, (C₁-C₆)alkoxy, (C₁-C₆)alkoxycarbonyl, nitro, halo, heteroaryl, —C(═O)NR^{v}R^{w}, —(OCH₂CH₂)_{y}—OR^{x}, —NR^{f}—SO₂—R^{c}, —NR^{f}—C(═O)—R^{c}, R^{m}, or Rⁿ;
each R^{c} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl;
each R^{d} and R^{e} is independently selected from H, (C₁-C₆)alkyl, (C₁-C₆)alkanoyl, (C₁-C₆)alkoxycarbonyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{d} and R^{e} together with the nitrogen to which they are attached form a aziridino, azetidino, morpholino, piperazino, pyrrolidino or piperidino; wherein any (C₁-C₆)alkyl, (C₃-C₆)cycloalkyl, (C₃-C₆)cycloalkyl(C₁-C₆)alkyl, aryl, heteroaryl, aryl(C₁-C₆)alkyl or heteroaryl(C₁-C₆)alkyl of R^{d} and R^{e} is optionally substituted with one or more groups independently selected from hydroxy, carboxy, and NR^{t}R^{u};
each R^{f} is H or (C₁-C₆)alkyl;
each R^{g} and R^{h} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{g} and R^{h} together with the nitrogen to which they are attached form a aziridino, azetidino, morpholino, piperazino, pyrrolidino or piperidino;
each R^{j} is independently selected from imidazoyl, piperazinyl, triazole, and piperazinyl that is optionally substituted with (C₁-C₆)alkyl;
each R^{k} is independently selected from —(C₁-C₆)alkyl that is substituted with a group selected from —NR^{d}R^{e}, —N⁺(R^{ac})₃Z⁻, —C(═NR^{c})—NR^{d}R^{e}, —NR^{f}—C(═NR^{c})—NR^{d}R^{e};
each R^{m} is independently selected from phenyl that is optionally substituted with one or more (C₁-C₆)alkyl, (C₁-C₆)alkoxy, trifluoromethyl, trifluoromethoxy, or halo;
each Rⁿ is independently selected from —(C₁-C₆)alkyl that is substituted with one or more groups independently selected from halo, hydroxy, and —NR^{g}R^{h};
each R^{t} and R^{u} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl; or R^{t} and R^{u} together with the nitrogen to which they are attached form a aziridino, azetidino, morpholino, piperazino, pyrrolidino or piperidino;
each R^{v} and R^{w} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl, wherein any (C₁-C₆)alkyl of R^{v} and R^{w} is optionally substituted with one or more NR^{aa}R^{ab}; or R^{v} and R^{w} together with the nitrogen to which they are attached form a aziridino, azetidino, morpholino, piperazino, pyrrolidino or piperidino;
each R^{x} is H or (C₁-C₆)alkyl;
each R^{aa} and R^{ab} is H or (C₁-C₆)alkyl;
each R^{ac} is independently selected from H, (C₁-C₆)alkyl, (C₃-C₆)cycloalkyl, (C₃-C₆)cycloalkyl(C₁-C₆)alkyl, aryl, heteroaryl, aryl(C₁-C₆)alkyl and heteroaryl(C₁-C₆)alkyl;
X is a direct bond or —C≡C—;
y is 1, 2, 3, 4, 5, or 6; and
each Z⁻ is independently a suitable counterion;
or a pharmaceutically acceptable salt thereof.

2. The method of claim 1 wherein ring A is phenyl, which is substituted with one or more R¹ and which is optionally substituted with one or more R^{b}.

3. The method of claim 1 wherein ring A is thiazolyl, which is substituted with one or more R¹ and which is optionally substituted with one or more R^{b}.

4. The method of claim 1 wherein R¹ is methyl, phenyl, tert-butyl, bromo, cyclohexyl, thiazolyl, biphenyl, thiazol-2-ylaminocarbonyl, or cyclopropyl.

5. The method of claim 1 wherein B is phenyl substituted with one or more R^{a} and optionally substituted with one or more R^{b}.

6. The method of claim 1 wherein B is pyridyl substituted with one or more R^{a} and optionally substituted with one or more R^{b}.

7. The method of claim 1 wherein B is thiazolyl substituted with one or more R^{a} and optionally substituted with one or more R^{b}.

8. The method of claim 1 wherein R^{a} is —N⁺(CH₃)₃Z⁻, —CH₂—N═C(NH₂)₂, —C(H)═N—NH—C(═NH)—NH₂, —C(CH₃)H—NH—C(═NH)—NH₂, 2-(1H-imidazol-1-yl)ethoxy, 1H-imidazol-1-ylmethyl, —O—CH₂—CH₂—NH—C(═NH)—NH₂, —CH₂—CH₂—N═C(NH₂)₂, —CH₂—CH₂—N⁺H₃Z⁻, 4-methylpiperazin-1-ylmethyl, aminomethyl, dimethylaminomethyl, 2-aminoethyl, N-but-2-ylaminomethyl, morpholinomethyl, 1-amino-1-methylethyl, —CH₂—NH—C(═NH)—NH—C(═O)OC(CH₃)₃, —CH₂—NH—NH—C(═NH)—NH₂, 1H-1,2,4-triazol-1-ylmethyl, —CH₂—N(CH(CH₃)CH₂CH₃)—C(═NH)—NH₂, —C(═O)—NH—C(═NH)—NH₂

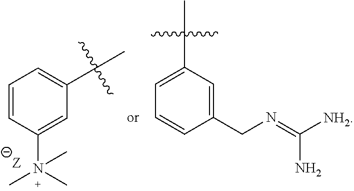

9. The method of claim 1 wherein each R^{b} is selected from methoxy, methyl, N,N-dimethylaminomethyl, bromo, 4-tert-butylphenyl, 4-trifluoromethoxy-2-methoxyphenyl, nitro, amino, methylsulfonylamino, methylcarbonylamino, hydroxymethyl, 2-(N,N-diethylamino)ethylaminocarbonyl, methoxy, —(OCH₂CH₂)₄—OCH₃, 2,2-dibromoethyl, thiazol-2-ylaminocarbonyl, and methoxycarbonyl.

10. The method of claim 1 wherein each R^{b} is selected from methoxy, N,N-dimethylaminomethyl, bromo, 4-tert-butylphenyl, 4-trifluoromethoxy-2-methoxyphenyl, nitro, and methoxycarbonyl.

11. The method of claim 1 wherein A is selected from:

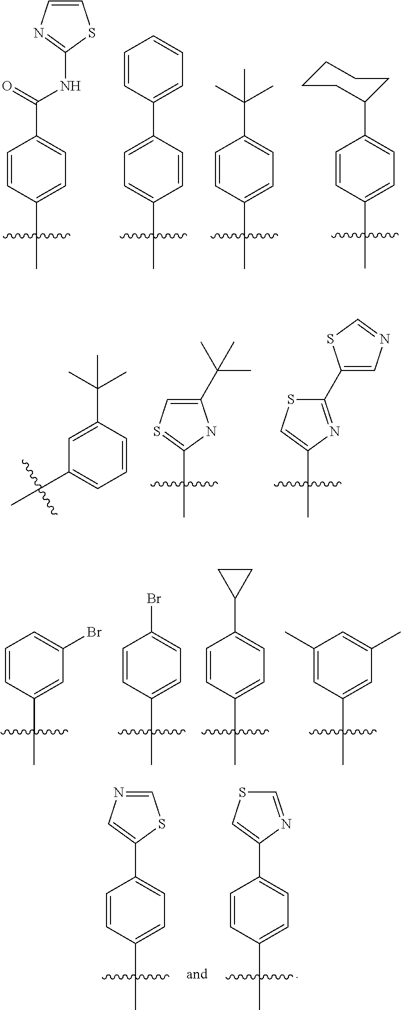

12. The method of claim 1 wherein the compound of formula I is selected from: 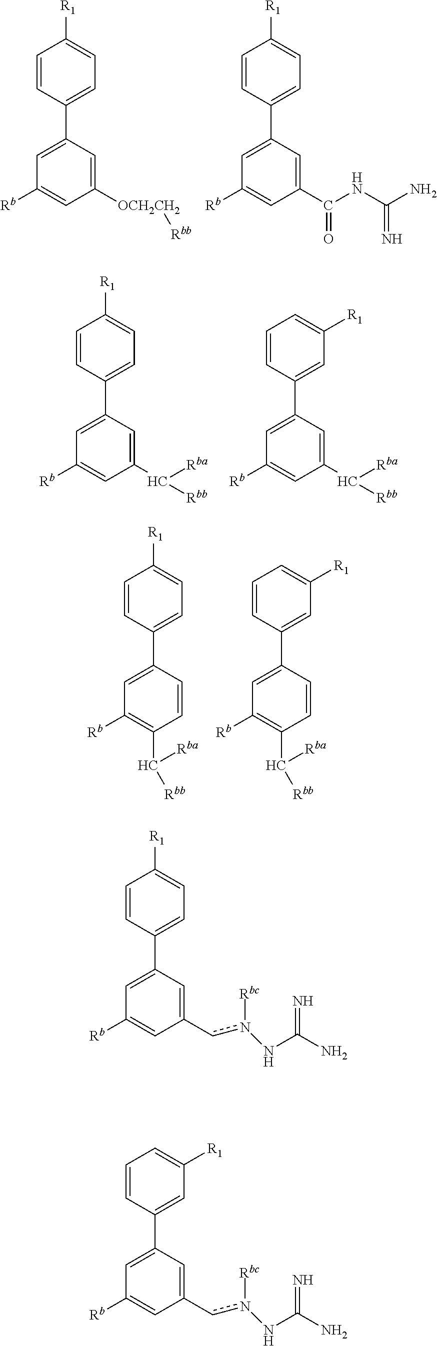 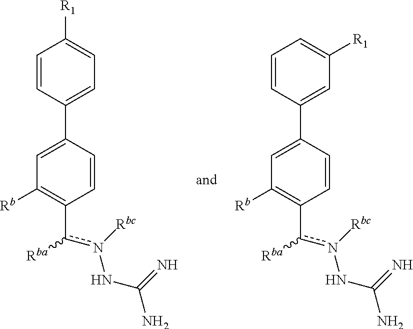
wherein:
the bond represented by --- is absent or is present to form a double bond;
each R^{ba} is H or methyl;
each R^{bb} is —NH—C(═NH)NH₂, —C(═NH)NH₂, or —CH₂NH₂; and
each ^{bc} is H or methyl, or is absent when the bond represented by --- is present.

13. The method of claim 12 wherein each R¹ is independently selected from cyclopropyl, tert-butyl, bromo, 4-tert-butylphenyl, and phenyl that is substituted at the 4-position with halo.

14. The method of claim 12 wherein each R^{b} is independently selected from H, chloro, bromo, fluoro, —NHSO₂H, and methoxy.

15. The method of claim 1 wherein the compound of formula I is selected from: 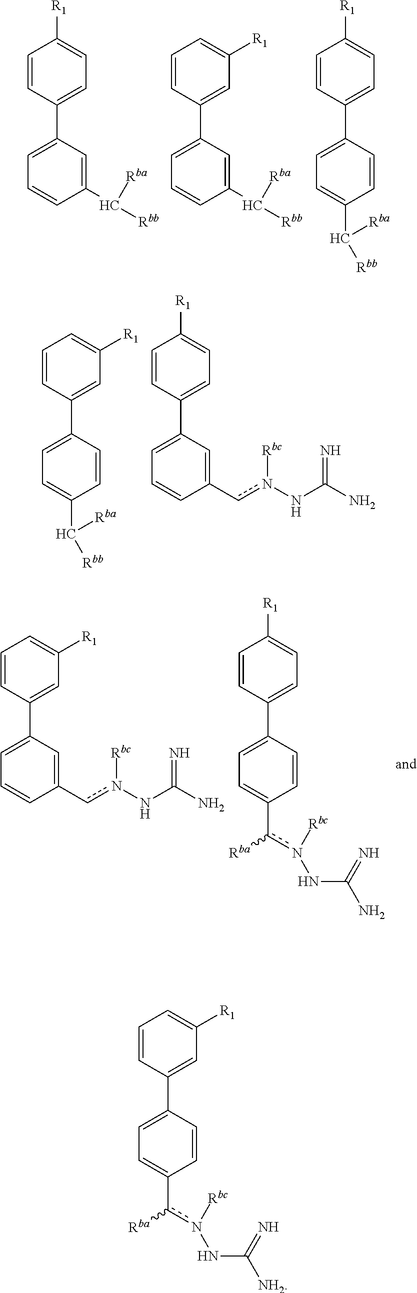
wherein:
the bond represented by --- is absent or is present to form a double bond;
each R^{ba} is H or methyl;
each R^{bb} is —NH—C(═NH)NH₂, —C(═NH)NH₂, or —CH₂NH₂; and
each ^{bc} is H or methyl, or is absent when the bond represented by --- is present.

16. The method of claim 12 wherein each R¹ is independently selected from cyclopropyl, tert-butyl, bromo, 4-tert-butylphenyl, and phenyl that is substituted at the 4-position with halo.

17. The method of claim 1 wherein a compound selected from 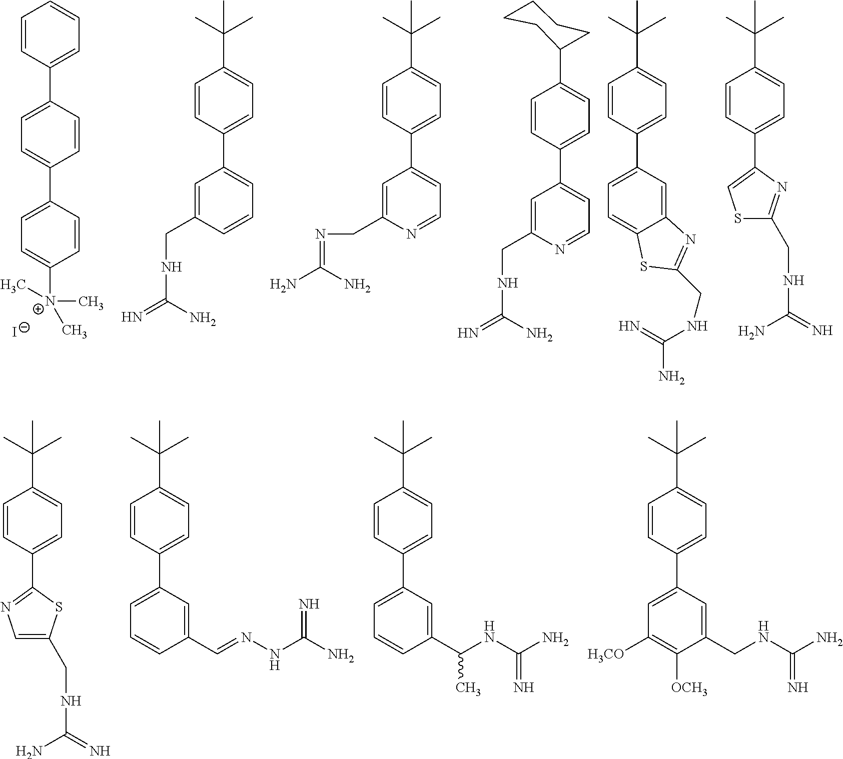 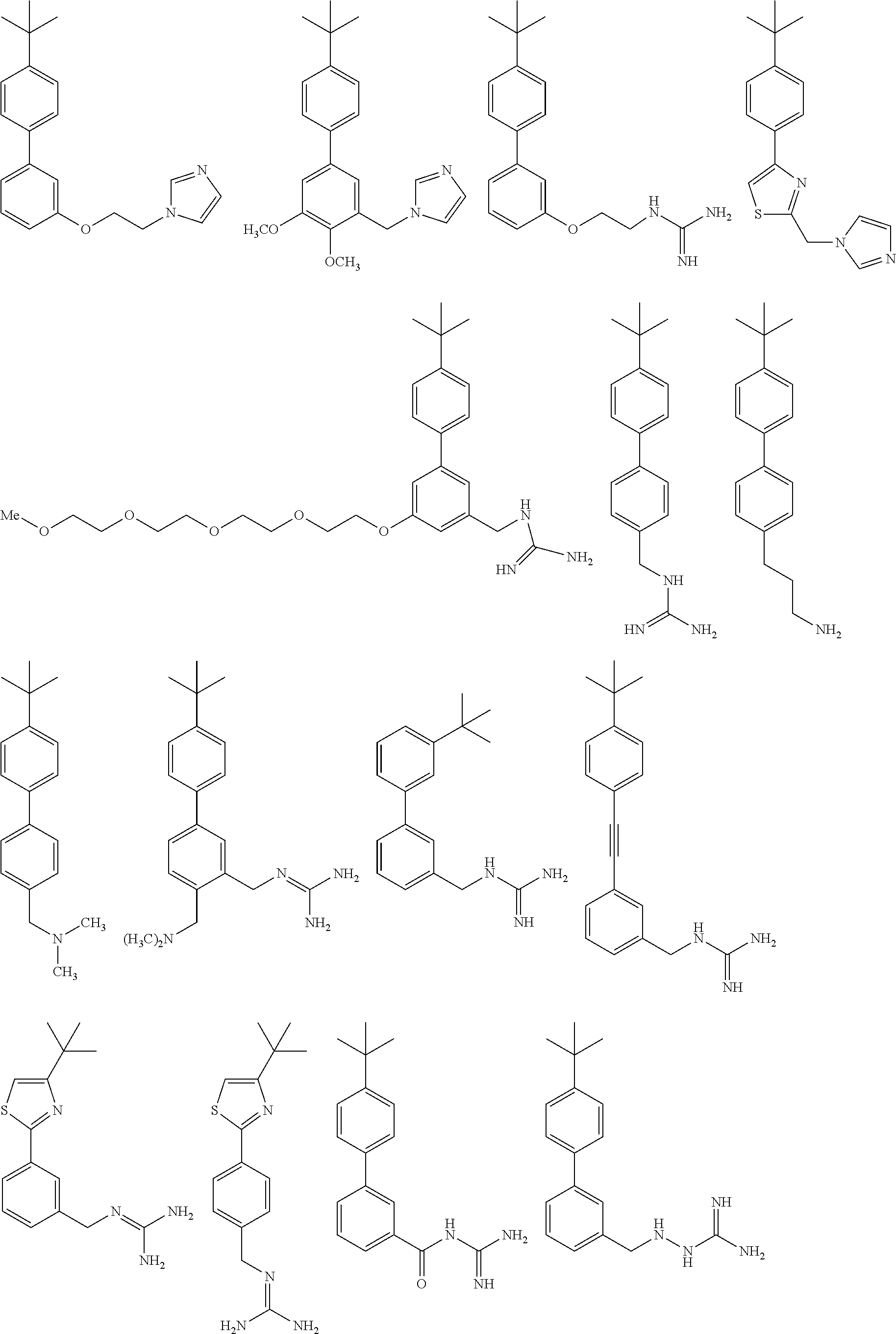 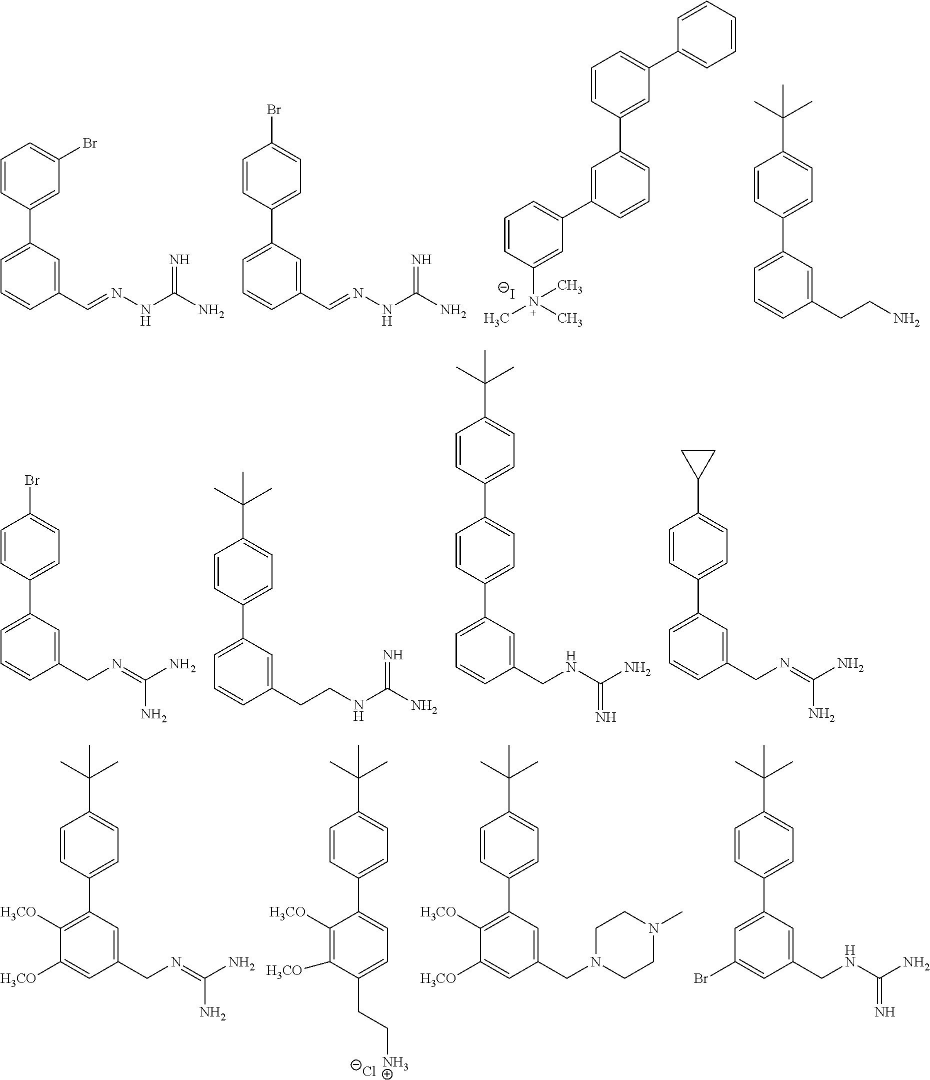 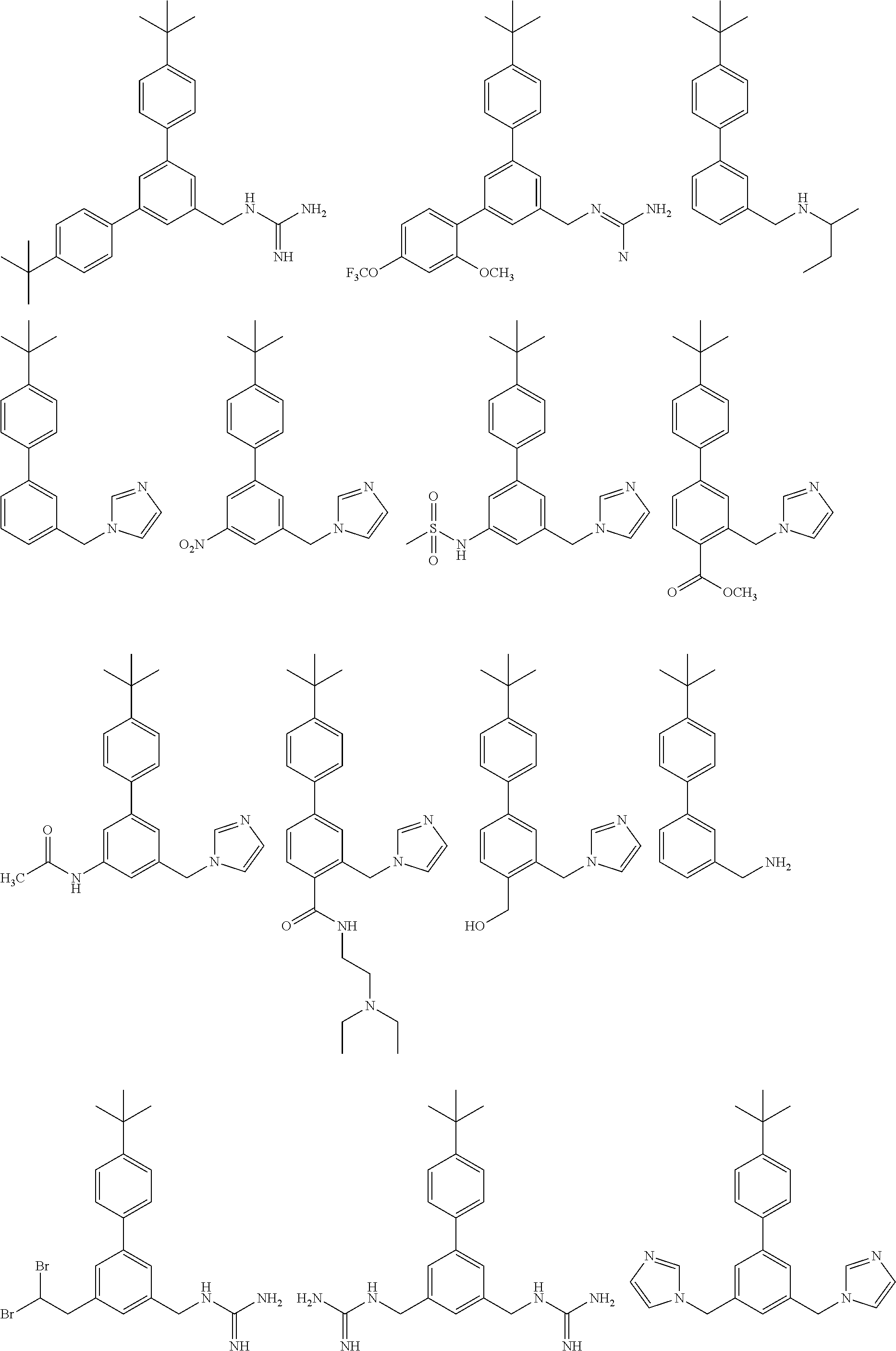 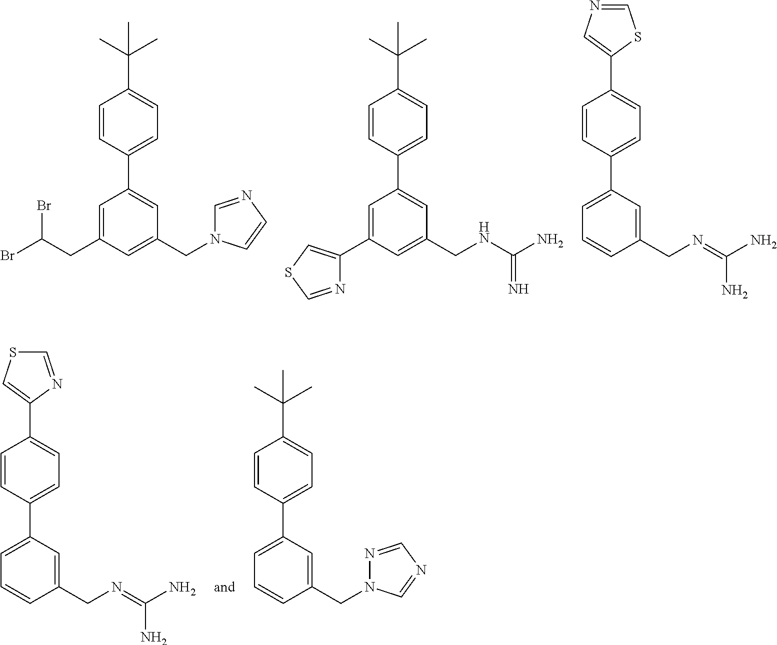
and pharmaceutically acceptable salts thereof is administered.

18. The method of claim 1 wherein the bacterial infection is a Gram-negative bacterial strain infection.

19. The method of claim 18 wherein the Gram-negative bacterial strain is selected from the group consisting of Escherchia coli, Caulobacter crescentus, Pseudomonas aeruginosa, Agrobacterium tumefaciens, Branhamella catarrhalis, Citrobacter diversus, Enterobacter aerogenes, Enterobacter cloacae, Enterobacter sakazakii, Enterobacter asburiae, Pantoea agglomerans, Klebsiella pneumoniae, Klebsiella oxytoca, Klebsiella rhinoscleromatis, Proteus mirabilis, Salmonella typhimurium, Salmonella enteriditis, Serratia marcescens, Shigella sonnei, Neisseria gonorrhoeae, Acinetobacter baumannii, Acinetobacter calcoaceticus, Acinetobacter lwoffi, Salmonella enteriditis, Fusobacterium nucleatum, Veillonella parvula, Bacteroides forsythus, Actinobacillus actinomycetemcomitans, Aggregatibacter actinomycetemcomitans, Porphyromonas gingivalis, Helicobacter pylori, Francisella tularensis, Yersinia pestis, Borrelia burgdorferi, Neisseria meningitidis and Haemophilus influenzae.

20. The method of claim 1 wherein the bacterial infection is a Gram-positive bacterial strain infection.

21. The method of claim 20 wherein the Gram-positive bacterial strain is selected from the group consisting of Staphylococcus aureus, Staphylococcus epidermidis, Staphylococcus saprophyticus, Streptococcus pyogenes, Streptococcus faecalis, Enterococcus faecalis, Enterococcus faecium, Bacillus subtilis, Micrococcus luteus, Mycobacterium tuberculosis, Bacillus anthraces, Bacillus cereus, Clostridium difficile, Propionibacterium acnes, Streptococcus mutans, Actinomyces viscosus, Actinomyces naeslundii, Streptococcus sanguis, Streptococcus pneumoniae and Streptococcus salivarius.

22. The method of claim 1 wherein the bacterial infection is a multiple drug-resistant bacterial strain infection.

23. The method of claim 22 wherein the multiple drug-resistance bacterial strain is selected from the group consisting of methicillin-resistant Staphylococcus aureus, vancomycin-resistant Enterococcus, multiple drug-resistant tuberculosis and multidrug-resistant Clostridium difficile.

24. (canceled)

25. A pharmaceutical composition comprising a compound of formula I as described in claim 1 or a pharmaceutically acceptable salt thereof, and a pharmaceutically acceptable vehicle.

26-28. (canceled)
